(19) United States
(12) Patent Application Publication
(10) Pub. No.: US 2025/0326817 A1
(43) Pub. Date: Oct. 23, 2025

(54) HERPES VIRUS ENTRY MEDIATOR PROTEINS AND METHODS OF USE THEREOF

(71) Applicants: Mitchell Kronenberg, La Jolla, CA (US); Steve Almo, Bronx, NY (US); Sarah Garrett, Bronx, NY (US); Weifeng Liu, Bronx, NY (US); Ting-Fang Chou, La Jolla, CA (US); LA JOLLA INSTITUTE FOR IMMUNOLOGY, La Jolla, CA (US); ALBERT EINSTEIN COLLEGE OF MEDICINE, INC., Bronx, NY (US)

(72) Inventors: Mitchell KRONENBERG, La Jolla, CA (US); Steve Almo, Bronx, NY (US); Sarah Garrett, Bronx, NY (US); Weifeng Liu, Bronx, NY (US); Ting-Fang Chou, La Jolla, CA (US)

(21) Appl. No.: 18/850,011

(22) PCT Filed: Mar. 24, 2023
(86) PCT No.: PCT/US2023/064952

§ 371 Date: Sep. 23, 2024

Related U.S. Application Data
(60) Provisional application No. 63/323,302, filed on Mar. 24, 2022.

(51) Int. Cl. C07K 14/705; A61K 38/00; A61K 40/11; A61K 40/31; A61K 40/42

(52) U.S. Cl. CPC C07K 14/70578; A61K 40/11; A61K 40/31; A61K 40/4215; A61K 38/00

(57) ABSTRACT

Provided herein, inter alia, are compositions including herpes virus entry mediator (HVEM) proteins and fragments thereof. The HVEM proteins provided herein may have modulated binding affinity to ligands and/or receptors compared to wild type HVEM proteins. The HVEM proteins provided herein are contemplated to be effective for treating diseases, particularly cancer and inflammatory diseases.

### CROSS-REFERENCE TO RELATED APPLICATIONS

This application claims priority to U.S. Provisional Application No. 63/323,302, filed Mar. 24, 2022, which is hereby incorporated by reference in its entirety and for all purposes.

### STATEMENT AS TO RIGHTS TO INVENTIONS MADE UNDER FEDERALLY SPONSORED RESEARCH AND DEVELOPMENT

This invention was made with government support under U01 AI125955 awarded by the National Institutes of Health. The government has certain rights in the invention.

### REFERENCE TO A SEQUENCE LISTING, A TABLE OR A COMPUTER PROGRAM LISTING APPENDIX SUBMITTED AS AN ASCII TEXT FILE

The contents of the electronic sequence listing (048513-514001WO_ST26.xml; Size 58,669 bytes; and Date of Creation: Mar. 24, 2023) is hereby incorporated by reference in its entirety.

### BACKGROUND OF THE INVENTION

The tumor necrosis factor receptor superfamily (TNFRSF) is a protein superfamily of cytokine receptors characterized by the ability to bind tumor necrosis factors (TNFs) via an extracellular cysteine-rich domain. Members of TNFRSF regulate diverse processes, but in several cases elucidation of these processes is hampered by promiscuous binding of receptors and ligands to multiple partners (1). One example is the herpes virus entry mediator (HVEM), or TNFRSF14, initially identified as important for cellular entry of the herpes simplex virus (HSV) through its recognition of HSV glycoprotein D (gD) (26, 44).

HVEM is a TNF receptor capable of binding to multiple ligands, and is thought to participate in signal transduction. For example, herpes virus entry mediator B and T lymphocyte attenuator (HVEM-BTLA) drives B cell growth and is thought to participate in pathology of B-cell lymphomas. HVEM is highly mutated in B-cell lymphomas, and may act as a ligand for B- and T-lymphocyte attenuator (BTLA). Therefore, modulating HVEM function may be important for treatment and/or prevention of lymphomas. HVEM proteins that do not engage non-target ligands is particularly important in order to prevent off-target effects.

Provided herein, inter alia, are solutions to these and other problems in the art.

### BRIEF SUMMARY

In an aspect is provided a herpes virus entry mediator (HVEM) protein or fragment thereof, including at least one of the following substitutions G72A, G72D, G72R, T73A, T73D, or V74D at a position corresponding to the amino acid sequence of SEQ ID NO: 11; or G72D, T73A, or V74E at a position corresponding the amino acid sequence of SEQ ID NO: 14.

In another aspect is provided a nucleic acid encoding an HVEM protein or fragment provided herein including embodiments thereof.

In an aspect is provided a Chimeric Antigen Receptor (CAR), the CAR including: a) an ectodomain of a HVEM protein or fragment provided herein including embodiments thereof, and b) a transmembrane domain.

In another aspect is provided a nucleic acid encoding a CAR provided herein including embodiments thereof.

In an aspect is provided a T-cell including a CAR provided herein including embodiments thereof.

In an aspect is provided a method of treating or preventing a disease in a subject in need thereof, the method including administering to the subject an effective amount of a HVEM protein or fragment thereof provided herein including embodiments thereof, a nucleic acid encoding an HVEM protein or fragment provided herein including embodiments thereof, a CAR provided herein including embodiments thereof, a nucleic acid encoding a CAR provided herein including embodiments thereof, or a T-cell provided herein including embodiments thereof.

In an aspect is provided a herpes virus entry mediator (HVEM) protein or fragment thereof, including at least one of the following substitutions: A76D, T82D, Y83A, T84D, T84F, H86D, N88A, N88D, G89D, L90A, L90D, L90R, L94A, L94D at a position corresponding to the amino acid sequence of SEQ ID NO: 11; or E76D, T82D, Y83A, 184D, 184F, H86D, N88A, N88D, G89D, L90A, L90D, L90R, L94A, or L94D at a position corresponding the amino acid sequence of SEQ ID NO: 14.

In another aspect is provided a nucleic acid encoding an HVEM protein or fragment provided herein including embodiments thereof.

In an aspect is provided a CAR, the CAR including: a) an ectodomain of a HVEM protein or fragment provided herein including embodiments thereof, and b) a transmembrane domain.

In another aspect is provided a nucleic acid encoding a CAR provided herein including embodiments thereof.

In an aspect is provided a T-cell including a CAR provided herein including embodiments thereof.

In an aspect a method of treating or preventing an autoimmune disorder in a subject in need thereof is provided, the method including administering to the subject an effective amount of a HVEM protein or fragment thereof provided herein including embodiments thereof, a nucleic acid encoding an HVEM protein or fragment provided herein including embodiments thereof, a CAR provided herein including embodiments thereof, a nucleic acid encoding a CAR provided herein including embodiments thereof, or a T-cell provided herein including embodiments thereof.

In an aspect a method of treating or preventing inflammation in a subject in need thereof is provided, the method including administering to the subject an effective amount of a HVEM protein or fragment thereof provided herein including embodiments thereof, a nucleic acid encoding an HVEM protein or fragment provided herein including embodiments thereof, a CAR provided herein including embodiments thereof, a nucleic acid encoding a CAR provided herein including embodiments thereof, or a T-cell provided herein including embodiments thereof.

### BRIEF DESCRIPTION OF THE DRAWINGS

FIGS. 1A-1F illustrate a crystal structure of human herpes virus entry mediator-homologous to lymphotoxin, exhibits inducible expression and competes with HSV glycoprotein D for binding to herpes virus entry mediator, a receptor expressed on T lymphocytes (HVEM:LIGHT) complex which exhibits a 3:3 stoichiometry. FIG. 1A: The analytical SEC trace of hHVEM and hLIGHT mixtures reveals a significant peak of the complex corresponding to the molecular weight around 100 kDa. The sodium dodecyl sulphate-polyacrylamide gel electrophoresis (SDS-PAGE) results indicate hHVEM and hLIGHT were purified to near homogeneity. Note that in the SDS gel, LIGHT trimers dissociate. FIGS. 1B-1C: The hHVEM is shown as a surface, and each coding region determinant (CRD) domain is separately indicated in the figure. The trimeric hLIGHT protein is shown as a ribbon in the figure. The side view (FIG. 1B) and bottom view (FIG. 1C) of the hHVEM:hLIGHT complex are shown. FIGS. 1D-1F: The detailed interaction interface between hHVEM and hLIGHT. The hydrogen bonds between hHVEM and hLIGHT are indicated as dashed lines. FIG. 1D illustrates that hHVEM A85 interacts with hLIGHT Y173. FIG. 1E shows that Human HVEM M98 interacts with hLIGHT R228 and hHVEM D100 interacts with hLIGHT R226. FIG. 1F illustrates that hHVEM residues 1128 and V129 interact with hLIGHT V152 and V196.

FIGS. 2A-2H illustrate overall structure of HVEM:LIGHT:CD160 ternary complex and critical interaction interfaces of HVEM binding to BTLA, CD160 and LIGHT. FIGS. 2A-2B: Structure of the hHVEM:hLIGHT:hCD160 ternary complex indicates hCD160 and hLIGHT can interact simultaneously with hHVEM. The side view (FIG. 2A) and the top/bottom views (FIG. 2B) of the ternary complex are shown. FIG. 2C: Structure of hHVEM:hBTLA (PDB entry 2AW2). FIG. 2D: Structure of hHVEM:hCD160 (PDB entry 6NG3). FIG. 2E: Structure of hHVEM:hLIGHT (PDB entry 4RSU). These structures indicate hBTLA and hCD160 bind to similar surfaces on hHVEM, whereas hLIGHT binds to a different surface on hHVEM. FIGS. 2F-2H: Detail binding interfaces between hHVEM and its binding ligands hBTLA (FIG. 2F), hCD160 (FIG. 2G) and Hlight (FIG. 2H).

FIGS. 3A-3D illustrate structure and mutagenesis screen of mHVEM. FIG. 3A: Structures of mHVEM, hHVEM and their comparison. The disulfide bonds of HVEM are shown as sticks. FIG. 3B: Sequence alignment of mHVEM (SEQ ID NO: 17) and hHVEM (SEQ ID NO: 50). The residues of hHVEM directly involved in the interface with hBTLA, hCD160, and Hlight are marked by triangles. The top row of triangles mark the interface with hBTLA, the middle row mark the interface with hCD160, and the lower row mark the interface with hLIGHT. FIG. 3C: The schematic illustrates two methods for determining the relative binding affinities of mHVEM mutants. The cell-cell method measures the percentages of double positive cells in the mixtures. The cell-protein method measures the percentages of green-fluorophore stained mHVEM-mCherry expressing cells. FIG. 3D: Relative binding affinities of mHVEM mutants with its ligands are shown in the table. Both mBTLA and mLIGHT binding to mHVEM was assessed by cell-cell method. The mCD160 binding to mHVEM was tested by cell-protein method. Error bars represent results from at least triplicates. All mHVEM mutants with ≥20% binding reduction to a particular query are shown to indicate their reduced affinities.

FIGS. 4A-4C illustrate that the engineered mHVEM mutants have binding selectivity to ligands. FIG. 4A: The relative binding affinities of mHVEM mutants with mBTLA, mCD160, and mLIGHT as measured by cell-cell or cell-protein methods. Error bars represent results from at least triplicates. The dashed line marks the averaged normalized affinities of wild-type mHVEM with mBTLA, mCD160, and mLIGHT. FIG. 4B: The locations of the mutated residues on mHVEM. mHVEM is shown as surface with CRD with G72, V74, H86 and L90 marked on the mHVEM surface. Ligands BTLA, CD160 and LIGHT are modeled based on the HVEM structures and are shown as labeled surfaces. FIG. 4C: The binding affinities of mHVEMWT (wild-type mHVEM), mHVEM-BT/160 (mHVEM G72R-V74A double mutein) and mHVEM-LIGHT (mHVEM H86D-L90A double mutein) with mBTLA, mCD160 and mLIGHT as measured by Octet bio-layer interferometry (BLI) technology.

FIGS. 5A-5F illustrate that mHVEM-^{LIGHT} mice were more susceptible to Y. enterocolitica infection. Male mice were infected with 1.0×108 Y. enterocolitica. KI=gene knockin. Survival curves of FIG. 5A mHVEM^{-LIGHT} mice and FIG. 5D: mHVEM-BT/160 mice. NS, not significant. *P=0.047 for Log-rank test. Changes in body weight (% of baseline) of FIG. 5B mHVEM^{-LIGHT} mice and FIG. 5E: mHVEM^{-BT/160} mice. *P<0.05; **P<0.01; ***P<0.001 (+/+vs KI/KI) or ^{#}P<0.05; ^{##}P<0.01; ^{###}P<0.001 (+/+vs KI/+) for two-way ANOVA with Bonferroni's multiple hypothesis correction. Representative hematoxylin and eosin (H&E) staining to detect necrotic areas and Warthin-Starry (WS) silver staining to detect bacteria in splenic and hepatic sections from the indicated mice at 7 days after infection. Scale bars, 100 μm. Light dotted lines indicate necrotic areas and dark dotted lines indicate Y. enterocolitica. Data shown are mean±s.e.m., and represent pooled results from at least two independent experiments having at least three mice per group in each experiment (n=6-12 mice per group; co-housed littermates). Data for FIG. 5C mHVEM^{-LIGHT} mice and FIG. 5F mHVEM^{-BT/160} mice are shown.

FIGS. 6A-6D illustrate susceptibility to αGalCer-induced liver injury in mHVEM-^{BT/160} mice. Mice were injected with 2 μg αGalCer by the retro-orbital route. FIG. 6A: Representative images of the liver 24 h after injection. Triangles indicate necrotic areas (bottom right panel). FIG. 6B: Representative H&E staining of hepatic sections from the indicated mice 24 h after injection. Dark dotted lines indicate the necrotic areas (bottom panels). Scale bars, 200 μm. FIGS. 6C-6D: Serum ALT activity at 16 and 24 h from the mHVEM^{-LIGHT} mice (FIG. 6C) and mHVEM^{-BT/160} mice (FIG. 6D). Data shown are mean±s.e.m.. *P<0.05; **P<0.01; ***P<0.001 for one-way ANOVA. Data represent pooled results from at least two independent experiments; each experiment labeled with symbols (n=4-10 mice per group; co-housed littermates).

FIG. 7 illustrates a diagram of the HVEM interaction network. HVEM can be activated by HSV gD, SALM5, CD160, BTLA, LIGHT, and for human HVEM, weakly by LTα. The interactions of HVEM with CD160 and BTLA result in bi-directional signaling to activate CD160 and BTLA as well as HVEM. LIGHT also engages LTβR besides HVEM, whereas these interactions can be neutralized by soluble DcR3 in humans.

FIGS. 8A-8E illustrate overall structure of hHVEM:hLIGHT complex and the binding interface between hHVEM and hLIGHT. FIGS. 8A-8B: One asymmetry unit contains 6 independent chains of hLIGHT and 6 independent chains of hHVEM forming two independent 3:3 hHVEM:hLIGHT complexes. Each chain is labeled in the figure. FIG. 8A: Side view of the two hHVEM:hLIGHT complexes in one asymmetry unit. FIG. 8B: Side view of the superimposition result of the two hHVEM:hLIGHT complexes. FIG. 8C: The overall structure of the hHVEM:hLIGHT complex (top left) and magnified view of one copy hHVEM binding to two adjacent hLIGHT monomers (bottom right). FIG. 8D: Magnified views of the binding interface between hHVEM and hLIGHT. The interaction interface of the “upper” region between hLIGHT and hHVEM (top panel). The interaction interface between the GH loop of hLIGHT and hHVEM (bottom panel). FIG. 8E: The interaction interface between the DE loop of hLIGHT and hHVEM (top panel). The interaction interface between the AA′ loop of hLIGHT and hHVEM (bottom panel).

FIGS. 9A-9G illustrate relative binding affinities of HVEM mutants with BTLA, CD160, and LIGHT. FIGS. 9A-9B: The hHVEM mutants were expressed on cell surface and were stained by hCD160, hBTLA, and hLIGHT proteins. The relative binding affinities were measured by flow cytometry. Error bars represent results from at least triplicates. FIG. 9A: shows the positions of the hHVEM mutation residues. FIG. 9B: shows the relative binding affinities of the hHVEM mutants. FIG. 9C: Superimposition of the hHVEM:hCD160 from the ternary complex with hHVEM:hCD160 complex alone (PDB entry 6NG3). FIG. 9D: Superimposition of the hHVEM:hLIGHT from the ternary complex with hHVEM:hLIGHT complex alone (PDB entry 4RSU). FIG. 9E: Relative binding affinities of mHVEM multiple-residue muteins with its ligands. Error bars represent results from at least triplicates. FIG. 9F: Representative flow cytometry results. The vertical axis is the mCherry fluorescence indicating mHVEM-expressing cells and the horizontal axis is the green fluorescence staining of fusion proteins or binding partner-expressing cells, as indicated. FIG. 9G: Ligand selective mHVEM mutein signaling. 293T cells were with mHVEM^{WT}, mHVEM^{-BT/160}, mHVEM^{-LIGHT}, or control (vector only) along with an NF-κB-driven luciferase (NF-κB-Luc) vector. mHVEM/NF-κB-Luc-expressed cells were co-cultured with control or mCD160- or mLIGHT-transfected 293T cells as indicated. Luciferase activity was measured after 18 h. Relative RLU (relative light units) is the ratio of Firefly luciferase luminescence to Renilla luciferase luminescence. Data shown are mean±s.d. ****P<0.0001 for two-way ANOVA. Data are representative of two independent experiments.

FIGS. 10A-10C illustrate normal surface HVEM expression in mHVEM mutant mice. FIG. 10A: Schematic of nucleotide sequences of the HVEM gene in mHVEM mutant mouse strains. Top panel shows the wild type sequence (SEQ ID NO: 55) and the G72R/V74A: mHVEM^{-BT/160} mutant (SEQ ID NO: 56) resulting in loss of BTLA and CD160 binding. Bottom panels shows the WT sequence (SEQ ID NO: 57) and the H86D/L90A mHVEM^{-LIGHT} mutant (SEQ ID NO: 58) resulting in loss of LIGHT binding that were generated by CRISPR-Cas9 editing of exon 3 of the Tnfrsf14 locus. Mutated amino acids are indicated in shaded boxes. FIG. 10B: HVEM surface expression level of splenic CD4⁺ T cells, ILC (CD3 Lin-CD90.2⁺), and iNKT cells (TCRβ⁺, CD1d tetramer⁺) from the indicated mice were determined by flow cytometry. HVEM-knockout (KO) mouse plays as a negative control of HVEM staining. KI=knockin allele. FIG. 10C: The morphometric scores of splenic or hepatic H&E sections from the indicated mice at 7 days after Y. enterocolitica infection were blinded and evaluated according to categories of phenotypes and lesions. Data shown are median *P<0.05 for two-way ANOVA.

FIG. 11 illustrates predicted maximum lengths of LIGHT, CD160 and BTLA stalk regions. The globular domains of LIGHT, CD160 and BTLA are shown as surface structures. The stalk regions that connect the extracellular globular domains to the transmembrane segments are shown as lines. The maximum lengths of the stalk regions are calculated as if they adopt the fully extended structures. The length of GPI-anchored CD160 stalk region in the figure does not include the GPI length. Amino acids are denoted as “aa” in the figure. This figure indicates that when human membrane LIGHT binds to HVEM, the longer stalk lengths of LIGHT may prevent BTLA and CD160 binding to HVEM.

FIGS. 12A-12D show cartoon structures of HVEM binding to FIG. 12A: tumor necrosis factor receptor superfamily ligands and FIG. 12B: Ig-superfamily ligands, and positions of residues in HVEM proteins that are important for binding FIG. 12C: BTLA/CD160 and FIG. 12D: LIGHT ligands. Substitution of G72 and/or V74 decreases binding affinity to BTLA/CD160. Substitution of H86 and/or L90 decrease binding affinity to LIGHT.

FIGS. 13A-13C show structures of WT mHvem and mutants G72R/V74A (FIG. 13A) and H86D/L90A (FIG. 13B), and analysis of their interactions with ligands using flow cytometry (FIG. 13C).

FIG. 14 illustrates binding affinities HVEM with their ligands as measured by Octet.

FIG. 15 shows residues important for LIGHT binding and demonstrates that modification of these residues result reduced LIGHT binding.

FIGS. 16A-16C illustrate results showing that the H86D/L90A mutant displays an intermediate phenotype of the heterozygote since HVEM is a trimeric protein. FIG. 16A illustrates a survival curve for mice expressing mHVEM^{-LIGHT} FIG. 16B illustrates disrupted binding between the mutant and LIGHT, and FIG. 16C illustrates a representative image of a section of spleen and liver stained with hematoxylin and eosin or Whartin-Starry silver in +/+ vs KI/KI mice.

FIGS. 17A-17C illustrate that selectivity and reduced binding to BTLA did not impair host defense. FIG. 17A illustrates a survival curve for mice expressing mHVEM^{-BT/160}. FIG. 17B illustrates disruption the the binding interface in the mHVEM^{-BT/160} mutant. FIG. 17C is a representative image of a section of a spleen stained with hematoxylin and eosin or Whartin-Starry silver in +/+ vs KI/KI mice.

FIG. 18 shows a ribbon diagram of a HVEM mutant with lost capability to bind BTLA/CD160.

FIGS. 19A-19B illustrate data showing that BTLA whole body gene KO mice have enhanced liver inflammation. Testing with HVEM muteins is on-going to demonstrate specificity. 2 ug/200 ul of alpha-GalCer was administered by retro-orbital injection. FIG. 19A shows representative images of mice liver undergoing alpha-GalCer administration. FIG. 19B is a graph showing alanine aminotransferase (ALT) activity in Btla f/f mice vs BTLA-KO mice.

### DETAILED DESCRIPTION OF THE INVENTION

Before the present invention is further described, it is to be understood that this invention is not strictly limited to particular embodiments described, as such may of course vary. It is also to be understood that the terminology used herein is for the purpose of describing particular embodiments only, and is not intended to be limiting, since the scope of the present invention will be limited only by the claims.

It must be noted that as used herein and in the appended claims, the singular forms “a,” “an,” and “the” include plural referents unless the context clearly dictates otherwise. It should further be understood that as used herein, the term “a” entity or “an” entity refers to one or more of that entity. For example, a nucleic acid molecule refers to one or more nucleic acid molecules. As such, the terms “a”, “an”, “one or more” and “at least one” can be used interchangeably. Similarly the terms “comprising”, “including” and “having” can be used interchangeably.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments are specifically embraced by the present invention and are disclosed herein just as if each and every combination was individually and explicitly disclosed. In addition, all sub-combinations are also specifically embraced by the present invention and are disclosed herein just as if each and every such sub-combination was individually and explicitly disclosed herein.

It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about means the specified value.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amio acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine.; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. One skilled in the art will immediately recognize the identity and location of residues corresponding to a specific position in a protein (e.g., HVEM) in other proteins with different numbering systems. For example, by performing a simple sequence alignment with a protein (e.g., HVEM) the identity and location of residues corresponding to specific positions of the protein are identified in other protein sequences aligning to the protein. For example, a selected residue in a selected protein corresponds to glutamic acid at position 138 when the selected residue occupies the same essential spatial or other structural relationship as a glutamic acid at position 138. In some embodiments, where a selected protein is aligned for maximum homology with a protein, the position in the aligned selected protein aligning with glutamic acid 138 is the to correspond to glutamic acid 138. Instead of a primary sequence alignment, a three-dimensional structural alignment can also be used, e.g., where the structure of the selected protein is aligned for maximum correspondence with the glutamic acid at position 138, and the overall structures compared. In this case, an amino acid that occupies the same essential position as glutamic acid 138 in the structural model is the to correspond to the glutamic acid 138 residue.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M)
(see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2: 482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

A “ligand” refers to an agent, e.g., a polypeptide or other molecule, capable of binding to a receptor, antibody, antibody variant, antibody region or fragment thereof.

For specific proteins described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

The term “HVEM protein” or “HVEM” as used herein includes any of the recombinant or naturally-occurring forms of Tumor necrosis factor receptor superfamily member 14, also known as Herpes virus entry mediator A, NHveA, tumor necrosis factor receptor-like 2, TR2, CD270, or variants or homologs thereof that maintain HVEM activity (e.g. within at least 50%, 80%, 90%, 95%, 96%, 97%, 98%, 99% or 100% activity compared to HVEM). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring HVEM protein. In embodiments, the HVEM protein is substantially identical to the protein identified by SEQ ID NO: 11. In embodiments, the HVEM protein is substantially identical to the protein identified by SEQ ID NO: 14. In embodiments, the HVEM protein is substantially identical to the protein identified by SEQ ID NO: 16. In embodiments, the HVEM protein is substantially identical to the protein identified by SEQ ID NO: 13. In embodiments, the HVEM protein is substantially identical to the protein identified by SEQ ID NO: 17. In embodiments, the HVEM protein is substantially identical to the protein identified by SEQ ID NO: 60. In embodiments, the HVEM protein is substantially identical to the protein identified by the UniProt reference number Q80WM9 or a variant or homolog having substantial identity thereto. In embodiments, the HVEM protein is substantially identical to the protein identified by the UniProt reference number Q92956 or a variant or homolog having substantial identity thereto.

The term “LIGHT protein” or “LIGHT” as used herein includes any of the recombinant or naturally-occurring forms of herpes virus entry mediator ligand, also known as tumor necrosis factor ligand superfamily member 14, HVEM-L, or variants or homologs thereof that maintain LIGHT activity (e.g. within at least 50%, 80%, 90%, 95%, 96%, 97%, 98%, 99% or 100% activity compared to LIGHT). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring LIGHT protein. In embodiments, the LIGHT protein is substantially identical to the protein identified by the UniProt reference number O43557 or a variant or homolog having substantial identity thereto. In embodiments, the LIGHT protein is substantially identical to the protein identified by the UniProt reference number Q92956 or a variant or homolog having substantial identity thereto. In embodiments, the LIGHT protein is substantially identical to the protein identified by the UniProt reference number Q9QYH9 or a variant or homolog having substantial identity thereto. In embodiments, the LIGHT protein is substantially identical to the protein identified by the UniProt reference number Q80WM9 or a variant or homolog having substantial identity thereto.

The term “BTLA protein” or “BTLA” as used herein includes any of the recombinant or naturally-occurring forms of B- and T-lymphocyte attenuator, also known as B- and T-lymphocyte-associated protein, CD272, or variants or homologs thereof that maintain BTLA activity (e.g. within at least 50%, 80%, 90%, 95%, 96%, 97%, 98%, 99% or 100% activity compared to BTLA). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring BTLA protein. In embodiments, the BTLA protein is substantially identical to the protein identified by Q7Z6A9 or a variant or homolog having substantial identity thereto. In embodiments, the HVEM protein is substantially identical to the protein identified by Q7TSA3 or a variant or homolog having substantial identity thereto.

The term “CD160 protein” or “CD160” as used herein includes any of the recombinant or naturally-occurring forms of CD160, also known as natural killer cell receptor BY55, or variants or homologs thereof that maintain CD160 activity (e.g. within at least 50%, 80%, 90%, 95%, 96%, 97%, 98%, 99% or 100% activity compared to CD160). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD160 protein. In embodiments, the CD160 protein is substantially identical to the protein identified by 095971 or a variant or homolog having substantial identity thereto. In embodiments, the CD160 protein is substantially identical to the protein identified by O88875 or a variant or homolog having substantial identity thereto.

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

A “label” or a “detectable moiety” is a composition detectable by spectroscopic, photochemical, biochemical, immunochemical, chemical, or other physical means. For example, useful labels include 32P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any appropriate method known in the art for conjugating an antibody to the label may be employed, e.g., using methods described in Hermanson, Bioconjugate Techniques 1996, Academic Press, Inc., San Diego.

When the label or detectable moiety is a radioactive metal or paramagnetic ion, the agent may be reacted with another long-tailed reagent having a long tail with one or more chelating groups attached to the long tail for binding to these ions. The long tail may be a polymer such as a polylysine, polysaccharide, or other derivatized or derivatizable chain having pendant groups to which the metals or ions may be added for binding. Examples of chelating groups that may be used according to the disclosure include, but are not limited to, ethylenediaminetetraacetic acid (EDTA), diethylenetriaminepentaacetic acid (DTPA), DOTA, NOTA, NETA, TETA, porphyrins, polyamines, crown ethers, bis-thiosemicarbazones, polyoximes, and like groups. The chelate is normally linked to the PSMA antibody or functional antibody fragment by a group, which enables the formation of a bond to the molecule with minimal loss of immunoreactivity and minimal aggregation and/or internal cross-linking. The same chelates, when complexed with non-radioactive metals, such as manganese, iron and gadolinium are useful for MRI, when used along with the antibodies and carriers described herein. Macrocyclic chelates such as NOTA, DOTA, and TETA are of use with a variety of metals and radiometals including, but not limited to, radionuclides of gallium, yttrium and copper, respectively. Other ring-type chelates such as macrocyclic polyethers, which are of interest for stably binding nuclides, such as ²²³Ra for RAIT may be used. In certain embodiments, chelating moieties may be used to attach a PET imaging agent, such as an Al—¹⁸F complex, to a targeting molecule for use in PET analysis.

“Contacting” is used in accordance with its plain ordinary meaning and refers to the process of allowing at least two distinct species (e.g. a receptor and protein) to become sufficiently proximal to react, interact or physically touch. It should be appreciated; however, the resulting reaction product can be produced directly from a reaction between the added reagents or from an intermediate from one or more of the added reagents that can be produced in the reaction mixture. The term “contacting” may include allowing two species to react, interact, or physically touch, wherein the two species may be a protein as described herein and receptor or ligand of said protein. In some embodiments contacting includes allowing a protein described herein to interact with a receptor or ligand that is involved in a signaling pathway.

The terms “virus” or “virus particle” are used according to its plain ordinary meaning within Virology and refers to a virion including the viral genome (e.g. DNA, RNA, single strand, double strand), viral capsid and associated proteins, and in the case of enveloped viruses (e.g. herpesvirus), an envelope including lipids and optionally components of host cell membranes, and/or viral proteins.

The term “viral infection” or “viral disease” refers to a disease or condition that is caused by a virus. Non-limiting examples of viral infections include hepatic viral diseases (e.g., hepatitis A, B, C, D, E), herpes virus infection (e.g., HSV-1, HSV-2, herpes zoster), flavivirus infection, Zika virus infection, cytomegalovirus infection, a respiratory viral infection (e.g., adenovirus infection, influenza, severe acute respiratory syndrome, coronavirus infection (e.g., SARS-CoV-1, SARS-CoV-2, MERS-CoV, COVID-19, MERS)), a gastrointestinal viral infection (e.g., norovirus infection, rotavirus infection, astrovirus infection), an exanthematous viral infection (e.g., measles, shingles, smallpox, rubella), viral hemorrhagic disease (e.g., Ebola, Lassa fever, dengue fever, yellow fever), a neurologic viral infection (e.g., West Nile viral infection, polio, viral meningitis, viral encephalitis, Japanese enchephalitis, rabies), and human papilloma viral infection.

The term “bacterial infection” or “bacterial disease” refers to a disease or condition that is caused by a bacteria. In embodiments, the bacterial infection is caused by Yersina enterocolita.

The term “replicate” is used in accordance with its plain ordinary meaning and refers to the ability of a cell or virus to produce progeny. A person of ordinary skill in the art will immediately understand that the term replicate when used in connection with DNA, refers to the biological process of producing two identical replicas of DNA from one original DNA molecule. In the context of a virus, the term “replicate” includes the ability of a virus to replicate (duplicate the viral genome and packaging said genome into viral particles) in a host cell and subsequently release progeny viruses from the host cell, which results in the lysis of the host cell. A “replication-competent” virus as provided herein refers to a virus that is capable of replicating in a cell (e.g., a cancer cell).

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include, but are not limited to, yeast cells and cells derived from plants and animals, for example mammalian, insect (e.g., spodoptera) and human cells.

As defined herein, the term “inhibition”, “inhibit”, “inhibiting” and the like in reference to cell proliferation (e.g., cancer cell proliferation) means negatively affecting (e.g., decreasing proliferation) or killing the cell. In some embodiments, inhibition refers to reduction of a disease or symptoms of disease (e.g., cancer, cancer cell proliferation). Thus, inhibition includes, at least in part, partially or totally blocking stimulation, decreasing, preventing, or delaying activation, or inactivating, desensitizing, or down-regulating signal transduction or enzymatic activity or the amount of a protein. Similarly an “inhibitor” is a compound or protein that inhibits a receptor or another protein, e.g., by binding, partially or totally blocking, decreasing, preventing, delaying, inactivating, desensitizing, or down-regulating activity (e.g., a receptor activity or a protein activity).

As defined herein, the term “inhibition”, “inhibit”, “inhibiting” and the like in reference to a protein-inhibitor interaction means negatively affecting (e.g. decreasing) the activity or function of the protein (e.g. HVEM protein, a receptor thereof, a ligand thereof) relative to the activity or function of the protein in the absence of the inhibitor. In embodiments, inhibition means negatively affecting (e.g. decreasing) the concentration or levels of a protein relative to the concentration or level of the protein in the absence of the inhibitor. In embodiments, inhibition refers to reduction of a disease or symptoms of disease. In embodiments, inhibition refers to a reduction in the activity of HVEM a receptor thereof, or a ligand thereof. Thus, inhibition includes, at least in part, partially or totally blocking stimulation, decreasing, preventing, or delaying activation, or inactivating, desensitizing, or down-regulating signal transduction or enzymatic activity or the amount of HVEM, a receptor thereof, or a ligand thereof. In embodiments, inhibition refers to a reduction of activity of HVEM resulting from a direct interaction (e.g. an inhibitor binds to HVEM). In embodiments, inhibition refers to a reduction of activity of HVEM from an indirect interaction (e.g. an inhibitor binds to an HVEM receptor, an HVEM ligand, etc.).

Thus, the terms “inhibitor,” “repressor” or “antagonist” or “downregulator” interchangeably refer to a substance capable of detectably decreasing the expression or activity of a given gene or protein (e.g. HVEM protein). The antagonist can decrease HVEM expression or activity 10%, 20%, 30%, 40%, 50%, 60%, 70%, 80%, 90% or more in comparison to a control in the absence of the antagonist. In certain instances, HVEM expression or activity is 1.5-fold, 2-fold, 3-fold, 4-fold, 5-fold, 10-fold or lower than the expression or activity in the absence of the antagonist.

As defined herein, the term “activation”, “activate”, “activating”, “activator” and the like in reference to a protein-inhibitor interaction means positively affecting (e.g. increasing) the activity or function of the protein (e.g. HVEM protein, a receptor thereof, a ligand thereof) relative to the activity or function of the protein in the absence of the activator. In embodiments activation means positively affecting (e.g. increasing) the concentration or levels of the protein (e.g. HVEM protein, a receptor thereof, a ligand thereof) relative to the concentration or level of the protein in the absence of the activator. The terms may reference activation, or activating, sensitizing, or up-regulating signal transduction or enzymatic activity or the amount of a protein decreased in a disease. Thus, activation may include, at least in part, partially or totally increasing stimulation, increasing or enabling activation, or activating, sensitizing, or up-regulating signal transduction or enzymatic activity or the amount of a protein (e.g. HVEM protein, a receptor thereof, a ligand thereof) associated with a disease (e.g., a protein which is decreased in a disease relative to a non-diseased control). Activation may include, at least in part, partially or totally increasing stimulation, increasing or enabling activation, or activating, sensitizing, or up-regulating signal transduction or enzymatic activity or the amount of a protein

The terms “agonist,” “activator,” “upregulator,” etc. refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “control” or “standard control” refers to a sample, measurement, or value that serves as a reference, usually a known reference, for comparison to a test sample, measurement, or value. For example, a test sample can be taken from a patient suspected of having a given disease (e.g. cancer) and compared to a known normal (non-diseased) individual (e.g. a standard control subject). A standard control can also represent an average measurement or value gathered from a population of similar individuals (e.g. standard control subjects) that do not have a given disease (i.e. standard control population), e.g., healthy individuals with a similar medical background, same age, weight, etc. A standard control value can also be obtained from the same individual, e.g. from an earlier-obtained sample from the patient prior to disease onset. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant. One of skill will recognize that standard controls can be designed for assessment of any number of parameters (e.g. RNA levels, protein levels, specific cell types, specific bodily fluids, specific tissues, etc).

One of skill in the art can understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

The terms “immune response” and the like refer, in the usual and customary sense, to a response by an organism that protects against disease. The response can be mounted by the innate immune system or by the adaptive immune system, as well known in the art.

“T cells” or “T lymphocytes” as used herein are a type of lymphocyte (a subtype of white blood cell) that plays a central role in cell-mediated immunity. They can be distinguished from other lymphocytes, such as B cells and natural killer cells, by the presence of a T-cell receptor on the cell surface. T cells include, for example, natural killer T (NKT) cells, cytotoxic T lymphocytes (CTLs), regulatory T (Treg) cells, and T helper cells. Different types of T cells can be distinguished by use of T cell detection agents.

The terms “disease” or “condition” refer to a state of being or health status of a patient or subject capable of being treated with the compounds or methods provided herein. The disease may be an autoimmune disorder. The disease may be a cancer. The cancer may refer to a solid tumor malignancy. Solid tumor malignancies include malignant tumors that may be devoid of fluids or cysts. For example, the solid tumor malignancy may include breast cancer, ovarian cancer, pancreatic cancer, cervical cancer, gastric cancer, renal cancer, head and neck cancer, bone cancer, skin cancer or prostate cancer. In some further instances, “cancer” refers to human cancers and carcinomas, sarcomas, adenocarcinomas, lymphomas, leukemias, including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including acute myeloid leukemia (AML), ALL, and CML), or multiple myeloma.

As used herein, the term “autoimmune disease” refers to a disease or condition in which a subject's immune system has an aberrant immune response against a substance that does not normally elicit an immune response in a healthy subject. Examples of autoimmune diseases that may be treated with a compound, pharmaceutical composition, or method described herein include Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenia purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenia purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenia purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, or Wegener's granulomatosis (i.e., Granulomatosis with Polyangiitis (GPA).

The term “associated” or “associated with” in the context of a substance or substance activity or function associated with a disease (e.g. a protein associated disease, a cancer associated with HVEM activity, HVEM associated cancer, HVEM associated disease (e.g., cancer, inflammatory disease, autoimmune disease, or infectious disease)) means that the disease (e.g. cancer, inflammatory disease, autoimmune disease, or infectious disease) is caused by (in whole or in part), or a symptom of the disease is caused by (in whole or in part) the substance or substance activity or function. As used herein, what is described as being associated with a disease, if a causative agent, could be a target for treatment of the disease. For example, a cancer associated with HVEM activity or function or a HVEM associated disease (e.g., cancer, inflammatory disease, autoimmune disease, or infectious disease), may be treated with a HVEM modulator or HVEM inhibitor, in the instance where increased HVEM activity or function (e.g. signaling pathway activity) causes the disease (e.g., cancer, inflammatory disease, autoimmune disease, or infectious disease). For example, an inflammatory disease associated with HVEM activity or function or an HVEM associated inflammatory disease, may be treated with an HVEM modulator or HVEM inhibitor, in the instance where increased HVEM activity or function (e.g. signaling pathway activity) causes the disease.

A “therapeutic agent” as referred to herein, is a composition useful in treating or preventing a disease (e.g. cancer, autoimmune disorder). In embodiments, the therapeutic agent is an anti-cancer agent. “Anti-cancer agent” is used in accordance with its plain ordinary meaning and refers to a composition (e.g. compound, drug, antagonist, inhibitor, modulator) having antineoplastic properties or the ability to inhibit the growth or proliferation of cells. In embodiments, an anti-cancer agent is a chemotherapeutic. In embodiments, an anti-cancer agent is an agent identified herein having utility in methods of treating cancer. In embodiments, an anti-cancer agent is an agent approved by the FDA or similar regulatory agency of a country other than the USA, for treating cancer.

As used herein, “treating” or “treatment of” a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total.

“Treating” can also mean prolonging survival of a subject beyond that expected in the absence of treatment. “Treating” can also mean inhibiting the progression of the condition, disorder or disease, slowing the progression of the condition, disorder or disease temporarily, although in some instances, it involves halting the progression of the condition, disorder or disease permanently. As used herein the terms treatment, treat, or treating refers to a method of reducing the effects of one or more symptoms of a disease or condition characterized by expression of the protease or symptom of the disease or condition characterized by expression of the protease. Thus in the disclosed method, treatment can refer to a 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

The terms “treating” or “treatment” refers to any indicia of success in the therapy or amelioration of a disease, pathology or condition, including any objective or subjective parameter such as abatement; remission; diminishing of symptoms or making the pathology or condition more tolerable to the patient; slowing in the rate of degeneration or decline; making the final point of degeneration less debilitating; improving a patient's physical well-being. The treatment or amelioration of symptoms can be based on objective or subjective parameters; including the results of a physical examination. The term “treating” and conjugations thereof, may include prevention of a pathology, condition, or disease. In aspects, treating is preventing. In aspects, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will be appreciated that the effective dosage of an agent used for the treatment or prophylaxis may increase or decrease over the course of a particular treatment or prophylaxis regime. Changes in dosage may result and become apparent by standard diagnostic assays known in the art. In embodiments, the treating or treatment is not prophylactic treatment.

A “effective amount,” as used herein, is an amount sufficient for a compound to accomplish a stated purpose relative to the absence of the compound (e.g. achieve the effect for which it is administered, treat a disease, reduce a signaling pathway, or reduce one or more symptoms of a disease or condition). In these methods, the effective amount of the active agent (e.g., oncolytic virus, viral vector) described herein is an amount effective to accomplish the stated purpose of the method. An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control. For any compound described herein, the therapeutically effective amount can be initially determined from cell culture assays. Target concentrations will be those concentrations of active compound(s) that are capable of achieving the methods described herein, as measured using the methods described herein or known in the art. As is in the art, therapeutically effective amounts for use in humans can also be determined from animal models. For example, a dose for humans can be formulated to achieve a concentration that has been found to be effective in animals.

The terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In embodiments, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

As used herein, the term “pharmaceutically acceptable” is used synonymously with “physiologically acceptable” and “pharmacologically acceptable”. A pharmaceutical composition will generally comprise agents for buffering and preservation in storage, and can include buffers and carriers for appropriate delivery, depending on the route of administration.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Protein Compositions

Provided herein, inter alia, are herpes virus entry mediator (HVEM) proteins and fragments that have modulated binding activity to ligands and/or receptors thereof, compared to the wild type HVEM protein. The HVEM proteins and fragments provided herein are contemplated to bind selectively to specific receptors and/or ligands, compared to the wild type HVEM protein. For example, an HVEM protein may have decreased binding capabilities to a ligand, thereby resulting in selective binding to other ligands. For example, HVEM proteins provided herein have may have decreased binding affinity to the TNF ligand LIGHT protein, thereby allowing selective binding to CD160 and/or BTLA. For example, HVEM proteins provided herein may have decreased binding to CD160 and/or BTLA, thereby allowing selective binding to LIGHT. Thus, in an aspect is provided a herpes virus entry mediator (HVEM) protein or fragment thereof, including at least one of the following substitutions G72A, G72D, G72R, T73A, T73D, or V74D at a position corresponding to the amino acid sequence of SEQ ID NO: 11; or G72D, T73A, or V74E at a position corresponding the amino acid sequence of SEQ ID NO: 14.

In embodiments, the HVEM protein or fragment thereof includes a G72A substitution at a position corresponding to the amino acid sequence of SEQ ID NO: 11. In embodiments, the HVEM protein or fragment thereof includes a G72D substitution at a position corresponding to the amino acid sequence of SEQ ID NO: 11. In embodiments, the HVEM protein or fragment thereof includes a G72R substitution at a position corresponding to the amino acid sequence of SEQ ID NO: 11. In embodiments, the HVEM protein or fragment thereof includes a T73A substitution at a position corresponding to the amino acid sequence of SEQ ID NO: 11. In embodiments, the HVEM protein or fragment thereof includes a T73D substitution at a position corresponding to the amino acid sequence of SEQ ID NO: 11. In embodiments, the HVEM protein or fragment thereof includes a V74D substitution at a position corresponding to the amino acid sequence of SEQ ID NO: 11.

In embodiments, the HVEM protein or fragment thereof includes a G72D at a position corresponding the amino acid sequence of SEQ ID NO: 14. In embodiments, the HVEM protein or fragment thereof includes a T73A at a position corresponding the amino acid sequence of SEQ ID NO: 14. In embodiments, the HVEM protein or fragment thereof includes a V74E at a position corresponding the amino acid sequence of SEQ ID NO: 14.

In embodiments, the HVEM protein or fragment includes a G72A and a V74D substitution, a G72D and a V74A substitution, a G72D and a V74D substitution, a G72D and a V74R substitution, or a G72R and a V74A substitution at positions corresponding to the amino acid sequence of SEQ ID NO: 11. In embodiments, the HVEM protein or fragment includes a G72A and a V74D substitution at positions corresponding to the amino acid sequence of SEQ ID NO: 11. In embodiments, the HVEM protein or fragment includes a G72D and a V74A substitution at positions corresponding to the amino acid sequence of SEQ ID NO: 11. In embodiments, the HVEM protein or fragment includes a G72D and a V74D substitution at positions corresponding to the amino acid sequence of SEQ ID NO: 11. In embodiments, the HVEM protein or fragment includes a G72D and a V74R substitution at positions corresponding to the amino acid sequence of SEQ ID NO: 11. In embodiments, the HVEM protein or fragment includes a G72R and a V74A substitution at positions corresponding to the amino acid sequence of SEQ ID NO: 11.

In embodiments, the HVEM protein or fragment includes a G72D and a V74E substitution at positions corresponding to the amino acid sequence of SEQ ID NO: 14.

In embodiments, the HVEM protein or fragment thereof includes amino acid sequence of SEQ ID NO: 18, SEQ ID NO: 19, SEQ ID NO: 20, SEQ ID NO: 21, SEQ ID NO: 22, SEQ ID NO: 23, SEQ ID NO: 38, SEQ ID NO: 39, SEQ ID NO: 40, SEQ ID NO: 41, or SEQ ID NO: 42. In embodiments, the HVEM protein or fragment thereof includes amino acid sequence of SEQ ID NO: 18. In embodiments, the HVEM protein or fragment thereof includes amino acid sequence of SEQ ID NO: 19. In embodiments, the HVEM protein or fragment thereof includes amino acid sequence of SEQ ID NO: 20. In embodiments, the HVEM protein or fragment thereof includes amino acid sequence of SEQ ID NO: 21. In embodiments, the HVEM protein or fragment thereof includes amino acid sequence of SEQ ID NO: 22. In embodiments, the HVEM protein or fragment thereof includes amino acid sequence of SEQ ID NO: 23. In embodiments, the HVEM protein or fragment thereof includes amino acid sequence of SEQ ID NO: 38. In embodiments, the HVEM protein or fragment thereof includes amino acid sequence of SEQ ID NO: 39. In embodiments, the HVEM protein or fragment thereof includes amino acid sequence of SEQ ID NO: 40. In embodiments, the HVEM protein or fragment thereof includes amino acid sequence of SEQ ID NO: 41. In embodiments, the HVEM protein or fragment thereof includes amino acid sequence of SEQ ID NO: 42.

In embodiments, the HVEM protein or fragment thereof includes the amino acid sequence of SEQ ID NO: 38, SEQ ID NO: 39, SEQ ID NO: 40, SEQ ID NO: 41, or SEQ ID NO: 42. In embodiments, the HVEM protein or fragment thereof includes the amino acid sequence of SEQ ID NO: 38. In embodiments, the HVEM protein or fragment thereof includes the amino acid sequence of SEQ ID NO: 39. In embodiments, the HVEM protein or fragment thereof includes the amino acid sequence of SEQ ID NO: 40. In embodiments, the HVEM protein or fragment thereof includes the amino acid sequence of SEQ ID NO: 41. In embodiments, the HVEM protein or fragment thereof includes the amino acid sequence of SEQ ID NO: 42.

In embodiments, the HVEM protein or fragment thereof further includes a delivery vehicle. The term “delivery vehicle” or “carrier” refers to any support structure that brings about the transfer of a component of genetic material or protein. For example, a delivery vehicle may transfer a protein (e.g. a HVEM protein) into a cell. Genetic material includes but is not limited to DNA, RNA, proteins, and polypeptides comprising amino acids. Proteins and polypeptides include but are not limited to antibodies, antigens, ligands, and receptors. Thus, in an embodiment, the delivery vehicle is an antibody fragment. For example, the delivery vehicle may be an Fc domain. In embodiments, the delivery vehicle is a vector. In embodiments, the vector is a viral vector. In embodiments, the delivery vehicle is a virus. Examples of viral delivery vehicles include retroviruses, adenoviruses, adeno-associated viruses, pseudotyped viruses, replication competent viruses, and herpes simplex virus. In embodiments, the delivery vehicle is a virus capsid, liposome or liposomal vesicle, lipoplex, polyplex, dendrimer, macrophage, artificial chromosome, nanoparticle, polymer or hybrid particle (e.g. virosome). Delivery vehicles may have multiple surfaces and compartments for attachment and storage of components, including outer surfaces and inner compartments.

In embodiments, the delivery vehicle is a nanoparticle. Any nanoparticle known in the art for protein or nucleic acid delivery can be used for the invention described herein. Nanoparticles are particles between 1 and 100 nanometers in size. Recent dramatic advances in nanotechnology have led to the development of a variety of nanoparticles (NPs) that provide valuable tools. Numerous nanomaterials such as polymers, liposomes, protein based NPs and inorganic NPs have been developed and a variety of particles are currently being evaluated in clinical studies with promising initial results; and some liposomal NPs are approved by the FDA. One of the major advantages of using these NPs is that they offer targeted tissue/site delivery. Their small size allows NPs to escape through blood vessels at the target site through the leaky vascular structure (Enhanced permeability and retention effect). In addition to this passive mechanism, a variety of targeting moieties can be attached to NPs to confer active targeting capability. Exemplary nanoparticles that can be used for delivering compositions described herein include, but are not limited to, solid nanoparticles (e.g., metals such as silver, gold, iron, titanium), non-metal, lipid-based solids (e.g., liposome), polymers (e.g., polyethylenimene, dendrimer), suspensions of nanoparticles, or combinations thereof (e.g., polyethylenimene-liposome, dendrisome). Additional information about nanoparticles that can be used by the compositions described herein can be found in Coelho et al., N Engl J Med 2013; 369:819-29, Tabernero et al., Cancer Discovery, April 2013, Vol. 3, No. 4, pages 363-470, Zhang et al., WO2015089419 A2, and Zuris J A et al., Nat Biotechnol. 2015; 33 (1): 73-80, each of which is incorporated herein by reference.

In an aspect is provided a herpes virus entry mediator (HVEM) protein or fragment thereof, including at least one of the following substitutions: T82D, Y83A, T84D, T84F, H86D, N88A, N88D, G89D, L90A, L90D, L90R, L94A, L94D at a position corresponding to the amino acid sequence of SEQ ID NO: 11; or E76D, T82D, Y83A, 184D, 184F, H86D, N88A, N88D, G89D, L90A, L90D, L90R, L94A, or L94D at a position corresponding the amino acid sequence of SEQ ID NO: 14.

In embodiments, the HVEM protein or fragment thereof includes a T82D substitution at a position corresponding to the amino acid sequence of SEQ ID NO: 11. In embodiments, the HVEM protein or fragment thereof includes a Y83A substitution at a position corresponding to the amino acid sequence of SEQ ID NO: 11. In embodiments, the HVEM protein or fragment thereof includes a T84D substitution at a position corresponding to the amino acid sequence of SEQ ID NO: 11. In embodiments, the HVEM protein or fragment thereof includes a T84F substitution at a position corresponding to the amino acid sequence of SEQ ID NO: 11. In embodiments, the HVEM protein or fragment thereof includes a H86D substitution at a position corresponding to the amino acid sequence of SEQ ID NO: 11. In embodiments, the HVEM protein or fragment thereof includes a N88A substitution at a position corresponding to the amino acid sequence of SEQ ID NO: 11. In embodiments, the HVEM protein or fragment thereof includes a N88D substitution at a position corresponding to the amino acid sequence of SEQ ID NO: 11. In embodiments, the HVEM protein or fragment thereof includes a G89D substitution at a position corresponding to the amino acid sequence of SEQ ID NO: 11. In embodiments, the HVEM protein or fragment thereof includes a L90A substitution at a position corresponding to the amino acid sequence of SEQ ID NO: 11. In embodiments, the HVEM protein or fragment thereof includes a L90D substitution at a position corresponding to the amino acid sequence of SEQ ID NO: 11. In embodiments, the HVEM protein or fragment thereof includes a L90R substitution at a position corresponding to the amino acid sequence of SEQ ID NO: 11. In embodiments, the HVEM protein or fragment thereof includes a L94A substitution at a position corresponding to the amino acid sequence of SEQ ID NO: 11. In embodiments, the HVEM protein or fragment thereof includes a L94D substitution at a position corresponding to the amino acid sequence of SEQ ID NO: 11.

In embodiments, the HVEM protein or fragment thereof includes a E76D substitution at a position corresponding the amino acid sequence of SEQ ID NO: 14. In embodiments, the HVEM protein or fragment thereof includes a T82D substitution at a position corresponding the amino acid sequence of SEQ ID NO: 14. In embodiments, the HVEM protein or fragment thereof includes a Y83A substitution at a position corresponding the amino acid sequence of SEQ ID NO: 14. In embodiments, the HVEM protein or fragment thereof includes a 184D substitution at a position corresponding the amino acid sequence of SEQ ID NO: 14. In embodiments, the HVEM protein or fragment thereof includes a I84F substitution at a position corresponding the amino acid sequence of SEQ ID NO: 14. In embodiments, the HVEM protein or fragment thereof includes a H86D substitution at a position corresponding the amino acid sequence of SEQ ID NO: 14. In embodiments, the HVEM protein or fragment thereof includes a N88A substitution at a position corresponding the amino acid sequence of SEQ ID NO: 14. In embodiments, the HVEM protein or fragment thereof includes a N88D substitution at a position corresponding the amino acid sequence of SEQ ID NO: 14. In embodiments, the HVEM protein or fragment thereof includes a G89D substitution at a position corresponding the amino acid sequence of SEQ ID NO: 14. In embodiments, the HVEM protein or fragment thereof includes a L90A substitution at a position corresponding the amino acid sequence of SEQ ID NO: 14. In embodiments, the HVEM protein or fragment thereof includes a L90D substitution at a position corresponding the amino acid sequence of SEQ ID NO: 14. In embodiments, the HVEM protein or fragment thereof includes a L90R substitution at a position corresponding the amino acid sequence of SEQ ID NO: 14. In embodiments, the HVEM protein or fragment thereof includes a L94A substitution at a position corresponding the amino acid sequence of SEQ ID NO: 14. In embodiments, the HVEM protein or fragment thereof includes a L94D substitution at a position corresponding the amino acid sequence of SEQ ID NO: 14.

In embodiments, the HVEM protein or fragment thereof includes a H86A and L90A substitution, a H86D and L90A substitution, a H86D and L90D substitution, a H86D and L90R substitution, or a H86R and L90A substitution at positions corresponding to the amino acid sequence of SEQ ID NO: 11. In embodiments, the HVEM protein or fragment thereof includes a H86A and L90A substitution at positions corresponding to the amino acid sequence of SEQ ID NO: 11. In embodiments, the HVEM protein or fragment thereof includes a H86D and L90A substitution at positions corresponding to the amino acid sequence of SEQ ID NO: 11. In embodiments, the HVEM protein or fragment thereof includes a H86D and L90D substitution at positions corresponding to the amino acid sequence of SEQ ID NO: 11. In embodiments, the HVEM protein or fragment thereof includes a H86D and L90R substitution at positions corresponding to the amino acid sequence of SEQ ID NO: 11. In embodiments, the HVEM protein or fragment thereof includes a H86R and L90A substitution at positions corresponding to the amino acid sequence of SEQ ID NO: 11.

In embodiments, the HVEM protein or fragment thereof includes a H86D and a L90A substitution, a H86D and a L90D substitution, or a H86D and a L90R substitution at a position corresponding the amino acid sequence of SEQ ID NO: 14. In embodiments, the HVEM protein or fragment thereof includes a H86D and a L90A substitution at positions corresponding to the amino acid sequence of SEQ ID NO: 14. In embodiments, the HVEM protein or fragment thereof includes a H86D and a L90D substitution at positions corresponding to the amino acid sequence of SEQ ID NO: 14. In embodiments, the HVEM protein or fragment thereof includes a H86D and a L90R substitution at positions corresponding to the amino acid sequence of SEQ ID NO: 14.

In embodiments, the HVEM protein or fragment thereof includes the amino acid sequence of SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, or SEQ ID NO: 54. In embodiments, the HVEM protein or fragment thereof includes the amino acid sequence of SEQ ID NO: 24. In embodiments, the HVEM protein or fragment thereof includes the amino acid sequence of SEQ ID NO: 25. In embodiments, the HVEM protein or fragment thereof includes the amino acid sequence of SEQ ID NO: 26. In embodiments, the HVEM protein or fragment thereof includes the amino acid sequence of SEQ ID NO: 27. In embodiments, the HVEM protein or fragment thereof includes the amino acid sequence of SEQ ID NO: 28. In embodiments, the HVEM protein or fragment thereof includes the amino acid sequence of SEQ ID NO: 29. In embodiments, the HVEM protein or fragment thereof includes the amino acid sequence of SEQ ID NO: 30. In embodiments, the HVEM protein or fragment thereof includes the amino acid sequence of SEQ ID NO: 31. In embodiments, the HVEM protein or fragment thereof includes the amino acid sequence of SEQ ID NO: 32. In embodiments, the HVEM protein or fragment thereof includes the amino acid sequence of SEQ ID NO: 33. In embodiments, the HVEM protein or fragment thereof includes the amino acid sequence of SEQ ID NO: 34. In embodiments, the HVEM protein or fragment thereof includes the amino acid sequence of SEQ ID NO: 35. In embodiments, the HVEM protein or fragment thereof includes the amino acid sequence of SEQ ID NO: 36. In embodiments, the HVEM protein or fragment thereof includes the amino acid sequence of SEQ ID NO: 37. In embodiments, the HVEM protein or fragment thereof includes the amino acid sequence of SEQ ID NO: 54.

In embodiments, the HVEM protein or fragment thereof includes the amino acid sequence of SEQ ID NO: 43, SEQ ID NO: 44, SEQ ID NO: 45, SEQ ID NO: 46, or SEQ ID NO: 47. In embodiments, the HVEM protein or fragment thereof includes the amino acid sequence of SEQ ID NO: 43. In embodiments, the HVEM protein or fragment thereof includes the amino acid sequence of SEQ ID NO: 44. In embodiments, the HVEM protein or fragment thereof includes the amino acid sequence of SEQ ID NO: 45. In embodiments, the HVEM protein or fragment thereof includes the amino acid sequence of SEQ ID NO: 46. In embodiments, the HVEM protein or fragment thereof includes the amino acid sequence of SEQ ID NO: 47.

In embodiments, the HVEM protein or fragment thereof includes a A76D, a H86D, and a L90A substitution at a position corresponding to the sequence of SEQ ID NO: 11. In embodiments, the HVEM protein or fragment thereof includes a A76D, a H86D, and a L90D substitution at a position corresponding to the sequence of SEQ ID NO: 11.

In embodiments, the HVEM protein includes the sequence of SEQ ID NO: 48. In embodiments, the HVEM protein includes the sequence of SEQ ID NO: 49.

In embodiments, the HVEM protein or fragment thereof further includes a delivery vehicle.

In embodiments, the HVEM protein or fragment thereof has decreased binding affinity to CD160 compared to a wild type HVEM protein. In embodiments, the HVEM protein or fragment thereof has decreased binding affinity to BTLA and CD160 compared to a wild type HVEM protein. In embodiments, the HVEM protein or fragment thereof has decreased binding affinity to LIGHT compared to a wild type HVEM protein. In embodiments, the HVEM protein provided herein binds to one or more ligands as described in Table 1.

The ability of a protein (e.g. HVEM) to bind a ligand (e.g., LIGHT, CD160, BTLA, etc.) can be described by the equilibrium dissociation constant (K_{D}). The equilibrium dissociation constant (K_{D}) as defined herein is the ratio of the dissociation rate (K-off) and the association rate (K-on) of ligand to a HVEM protein. It is described by the following formula: K_{D}=K-off/K-on.

In embodiments, the HVEM protein binds LIGHT with an equilibrium dissociation constant (K_{D}) from about 1 uM to about 100 uM. In embodiments, the HVEM protein binds LIGHT with a K_{D} from about 5 uM to about 100 uM. In embodiments, the HVEM protein binds LIGHT with a K_{D} from about 10 uM to about 100 uM. In embodiments, the HVEM protein binds LIGHT with a K_{D} from about 15 uM to about 100 uM. In embodiments, the HVEM protein binds LIGHT with a K_{D} from about 20 uM to about 100 uM. In embodiments, the HVEM protein binds LIGHT with a K_{D} from about 25 uM to about 100 uM. In embodiments, the HVEM protein binds LIGHT with a K_{D} from about 30 uM to about 100 uM. In embodiments, the HVEM protein binds LIGHT with a K_{D} from about 35 uM to about 100 uM. In embodiments, the HVEM protein binds LIGHT with a K_{D} from about 40 uM to about 100 uM. In embodiments, the HVEM protein binds LIGHT with a K_{D} from about 45 uM to about 100 uM. In embodiments, the HVEM protein binds LIGHT with a K_{D} from about 50 uM to about 100 uM. In embodiments, the HVEM protein binds LIGHT with a K_{D} from about 55 uM to about 100 uM. In embodiments, the HVEM protein binds LIGHT with a K_{D} from about 60 uM to about 100 uM. In embodiments, the HVEM protein binds LIGHT with a K_{D} from about 65 uM to about 100 uM. In embodiments, the HVEM protein binds LIGHT with a K_{D} from about 70 uM to about 100 uM. In embodiments, the HVEM protein binds LIGHT with a K_{D} from about 75 uM to about 100 uM. In embodiments, the HVEM protein binds LIGHT with a K_{D} from about 80 uM to about 100 uM. In embodiments, the HVEM protein binds LIGHT with a K_{D} from about 85 uM to about 100 uM. In embodiments, the HVEM protein binds LIGHT with a K_{D} from about 90 uM to about 100 uM. In embodiments, the HVEM protein binds LIGHT with a K_{D} from about 95 uM to about 100 uM.

In embodiments, the HVEM protein binds LIGHT with a K_{D} from about 1 uM to about 95 uM. In embodiments, the HVEM protein binds LIGHT with a K_{D} from about 1 uM to about 90 uM. In embodiments, the HVEM protein binds LIGHT with a K_{D} from about 1 uM to about 85 uM. In embodiments, the HVEM protein binds LIGHT with a K_{D} from about 1 uM to about 80 uM. In embodiments, the HVEM protein binds LIGHT with a K_{D} from about 1 uM to about 75 uM. In embodiments, the HVEM protein binds LIGHT with a K_{D} from about 1 uM to about 70 uM. In embodiments, the HVEM protein binds LIGHT with a K_{D} from about 1 uM to about 65 uM. In embodiments, the HVEM protein binds LIGHT with a K_{D} from about 1 uM to about 60 uM. In embodiments, the HVEM protein binds LIGHT with a K_{D} from about 1 uM to about 55 uM. In embodiments, the HVEM protein binds LIGHT with a K_{D} from about 1 uM to about 50 uM. In embodiments, the HVEM protein binds LIGHT with a K_{D} from about 1 uM to about 45 uM. In embodiments, the HVEM protein binds LIGHT with a K_{D} from about 1 uM to about 40 uM. In embodiments, the HVEM protein binds LIGHT with a K_{D} from about 1 uM to about 35 uM. In embodiments, the HVEM protein binds LIGHT with a K_{D} from about 1 uM to about 30 uM. In embodiments, the HVEM protein binds LIGHT with a K_{D} from about 1 uM to about 25 uM. In embodiments, the HVEM protein binds LIGHT with a K_{D} from about 1 uM to about 20 uM. In embodiments, the HVEM protein binds LIGHT with a K_{D} from about 1 uM to about 15 uM. In embodiments, the HVEM protein binds LIGHT with a K_{D} from about 1 uM to about 10 uM. In embodiments, the HVEM protein binds LIGHT with a K_{D} from about 1 uM to about 5 uM. In embodiments, the HVEM protein binds LIGHT with a K_{D} of about 1 uM, 5 uM, 10 uM, 15 uM, 20 uM, 25 uM, 30 uM, 35 uM, 40 uM, 45 uM, 50 uM, 55 uM, 60 uM, 65 uM, 70 uM, 75 uM, 80 uM, 85 uM, 90 uM, 95 uM or 100 uM. In embodiments, the HVEM protein binds LIGHT with a K_{D} of about 31.7 uM. In embodiments, the HVEM protein binds LIGHT with a K_{D} of 31.7 uM.

In embodiments, the HVEM protein binds LIGHT with a K_{D} from about 0.05 uM to about 12.05 uM. In embodiments, the HVEM protein binds LIGHT with a K_{D} from about 0.55 uM to about 12.05 uM. In embodiments, the HVEM protein binds LIGHT with a K_{D} from about 1.05 uM to about 12.05 uM. In embodiments, the HVEM protein binds LIGHT with a K_{D} from about 1.55 uM to about 12.05 uM. In embodiments, the HVEM protein binds LIGHT with a K_{D} from about 2.05 uM to about 12.05 uM. In embodiments, the HVEM protein binds LIGHT with a K_{D} from about 2.55 uM to about 12.05 uM. In embodiments, the HVEM protein binds LIGHT with a K_{D} from about 3.05 uM to about 12.05 uM. In embodiments, the HVEM protein binds LIGHT with a K_{D} from about 3.55 uM to about 12.05 uM. In embodiments, the HVEM protein binds LIGHT with a K_{D} from about 4.05 uM to about 12.05 uM. In embodiments, the HVEM protein binds LIGHT with a K_{D} from about 4.55 uM to about 12.05 uM. In embodiments, the HVEM protein binds LIGHT with a K_{D} from about 5.05 uM to about 12.05 uM. In embodiments, the HVEM protein binds LIGHT with a K_{D} from about 5.55 uM to about 12.05 uM. In embodiments, the HVEM protein binds LIGHT with a K_{D} from about 6.05 uM to about 12.05 uM. In embodiments, the HVEM protein binds LIGHT with a K_{D} from about 6.55 uM to about 12.05 uM. In embodiments, the HVEM protein binds LIGHT with a K_{D} from about 7.05 uM to about 12.05 uM. In embodiments, the HVEM protein binds LIGHT with a K_{D} from about 7.55 uM to about 12.05 uM. In embodiments, the HVEM protein binds LIGHT with a K_{D} from about 8.05 uM to about 12.05 uM. In embodiments, the HVEM protein binds LIGHT with a K_{D} from about 8.55 uM to about 12.05 uM. In embodiments, the HVEM protein binds LIGHT with a K_{D} from about 9.05 uM to about 12.05 uM. In embodiments, the HVEM protein binds LIGHT with a K_{D} from about 9.55 uM to about 12.05 uM. In embodiments, the HVEM protein binds LIGHT with a K_{D} from about 10.05 uM to about 12.05 uM. In embodiments, the HVEM protein binds LIGHT with a K_{D} from about 10.55 uM to about 10.05 uM. In embodiments, the HVEM protein binds LIGHT with a K_{D} from about 11.05 uM to about 12.05 uM. In embodiments, the HVEM protein binds LIGHT with a K_{D} from about 11.55 uM to about 12.05 uM.

In embodiments, the HVEM protein binds LIGHT with a K_{D} from about 0.05 uM to about 11.55 uM. In embodiments, the HVEM protein binds LIGHT with a K_{D} from about 0.05 uM to about 11.05 uM. In embodiments, the HVEM protein binds LIGHT with a K_{D} from about 0.05 uM to about 10.55 uM. In embodiments, the HVEM protein binds LIGHT with a K_{D} from about 0.05 uM to about 10.05 uM. In embodiments, the HVEM protein binds LIGHT with a K_{D} from about 0.05 uM to about 9.55 uM. In embodiments, the HVEM protein binds LIGHT with a K_{D} from about 0.05 uM to about 9.05 uM. In embodiments, the HVEM protein binds LIGHT with a K_{D} from about 0.05 uM to about 8.55 uM. In embodiments, the HVEM protein binds LIGHT with a K_{D} from about 0.05 uM to about 8.05 uM. In embodiments, the HVEM protein binds LIGHT with a K_{D} from about 0.05 uM to about 7.55 uM. In embodiments, the HVEM protein binds LIGHT with a K_{D} from about 0.05 uM to about 7.05 uM. In embodiments, the HVEM protein binds LIGHT with a K_{D} from about 0.05 uM to about 6.55 uM. In embodiments, the HVEM protein binds LIGHT with a K_{D} from about 0.05 uM to about 6.05 uM. In embodiments, the HVEM protein binds LIGHT with a K_{D} from about 0.05 uM to about 5.55 uM. In embodiments, the HVEM protein binds LIGHT with a K_{D} from about 0.05 uM to about 5.05 uM. In embodiments, the HVEM protein binds LIGHT with a K_{D} from about 0.05 uM to about 4.55 uM. In embodiments, the HVEM protein binds LIGHT with a K_{D} from about 0.05 uM to about 4.05 uM. In embodiments, the HVEM protein binds LIGHT with a K_{D} from about 0.05 uM to about 3.55 uM. In embodiments, the HVEM protein binds LIGHT with a K_{D} from about 0.05 uM to about 3.05 uM. In embodiments, the HVEM protein binds LIGHT with a K_{D} from about 0.05 uM to about 2.55 uM. In embodiments, the HVEM protein binds LIGHT with a K_{D} from about 0.05 uM to about 2.05 uM. In embodiments, the HVEM protein binds LIGHT with a K_{D} from about 0.05 uM to about 1.55 uM. In embodiments, the HVEM protein binds LIGHT with a K_{D} from about 0.05 uM to about 1.05 uM. In embodiments, the HVEM protein binds LIGHT with a K_{D} from about 0.05 uM to about 0.55 uM. In embodiments, the HVEM protein binds LIGHT with a K_{D} of about 0.05 uM, 0.55 uM, 1.05 uM, 1.55 uM, 2.05 uM, 2.55 uM, 3.05 uM, 3.55 uM, 4.05 uM, 4.55 uM, 5.05 uM, 5.55 uM, 6.05 uM, 6.55 uM, 7.05 UM, 7.55 uM, 8.05 uM, 8.55 uM, 9.05 UM, 9.55 uM, 10.05 uM, 10.55 uM, 11.05 uM, 11.55 uM, or 12.05 uM. In embodiments, the HVEM protein binds LIGHT with a K_{D} of about 1.2 uM. In embodiments, the HVEM protein binds LIGHT with a K_{D} of 1.2 uM. In embodiments, the HVEM protein binds LIGHT with a K_{D} of about 0.7 uM. In embodiments, the HVEM protein binds LIGHT with a K_{D} of 0.7 uM. In embodiments, the HVEM protein binds LIGHT with a K_{D} of about 0.9 uM. In embodiments, the HVEM protein binds LIGHT with a K_{D} of 0.9 uM. In embodiments, the HVEM protein binds LIGHT with a K_{D} of about 1.8 uM. In embodiments, the HVEM protein binds LIGHT with a K_{D} of 1.8 uM. In embodiments, the HVEM protein binds LIGHT with a K_{D} of about 1.0 uM. In embodiments, the HVEM protein binds LIGHT with a K_{D} of 1.0 uM.

In embodiments, the HVEM protein binds LIGHT with an equilibrium dissociation constant (K_{D}) from about 1 nM to about 100 nM. In embodiments, the HVEM protein binds LIGHT with a K_{D} from about 5 nM to about 100 nM. In embodiments, the HVEM protein binds LIGHT with a K_{D} from about 10 nM to about 100 nM. In embodiments, the HVEM protein binds LIGHT with a K_{D} from about 15 nM to about 100 nM. In embodiments, the HVEM protein binds LIGHT with a K_{D} from about 20 nM to about 100 nM. In embodiments, the HVEM protein binds LIGHT with a K_{D} from about 25 nM to about 100 nM. In embodiments, the HVEM protein binds LIGHT with a K_{D} from about 30 nM to about 100 nM. In embodiments, the HVEM protein binds LIGHT with a K_{D} from about 35 nM to about 100 nM. In embodiments, the HVEM protein binds LIGHT with a K_{D} from about 40 nM to about 100 nM. In embodiments, the HVEM protein binds LIGHT with a K_{D} from about 45 nM to about 100 nM. In embodiments, the HVEM protein binds LIGHT with a K_{D} from about 50 nM to about 100 nM. In embodiments, the HVEM protein binds LIGHT with a K_{D} from about 55 nM to about 100 nM. In embodiments, the HVEM protein binds LIGHT with a K_{D} from about 60 nM to about 100 nM. In embodiments, the HVEM protein binds LIGHT with a K_{D} from about 65 nM to about 100 nM. In embodiments, the HVEM protein binds LIGHT with a K_{D} from about 70 nM to about 100 nM. In embodiments, the HVEM protein binds LIGHT with a K_{D} from about 75 nM to about 100 nM. In embodiments, the HVEM protein binds LIGHT with a K_{D} from about 80 nM to about 100 nM. In embodiments, the HVEM protein binds LIGHT with a K_{D} from about 85 nM to about 100 nM. In embodiments, the HVEM protein binds LIGHT with a K_{D} from about 90 nM to about 100 nM. In embodiments, the HVEM protein binds LIGHT with a K_{D} from about 95 nM to about 100 nM.

In embodiments, the HVEM protein binds LIGHT with a K_{D} from about 1 nM to about 95 nM. In embodiments, the HVEM protein binds LIGHT with a K_{D} from about 1 nM to about 90 nM. In embodiments, the HVEM protein binds LIGHT with a K_{D} from about 1 nM to about 85 nM. In embodiments, the HVEM protein binds LIGHT with a K_{D} from about 1 nM to about 80 nM. In embodiments, the HVEM protein binds LIGHT with a K_{D} from about 1 nM to about 75 nM. In embodiments, the HVEM protein binds LIGHT with a K_{D} from about 1 nM to about 70 nM. In embodiments, the HVEM protein binds LIGHT with a K_{D} from about 1 nM to about 65 nM. In embodiments, the HVEM protein binds LIGHT with a K_{D} from about 1 nM to about 60 nM. In embodiments, the HVEM protein binds LIGHT with a K_{D} from about 1 nM to about 55 nM. In embodiments, the HVEM protein binds LIGHT with a K_{D} from about 1 nM to about 50 nM. In embodiments, the HVEM protein binds LIGHT with a K_{D} from about 1 nM to about 45 nM. In embodiments, the HVEM protein binds LIGHT with a K_{D} from about 1 nM to about 40 nM. In embodiments, the HVEM protein binds LIGHT with a K_{D} from about 1 nM to about 35 nM. In embodiments, the HVEM protein binds LIGHT with a K_{D} from about 1 nM to about 30 nM. In embodiments, the HVEM protein binds LIGHT with a K_{D} from about 1 nM to about 25 nM. In embodiments, the HVEM protein binds LIGHT with a K_{D} from about 1 nM to about 20 nM. In embodiments, the HVEM protein binds LIGHT with a K_{D} from about 1 nM to about 15 nM. In embodiments, the HVEM protein binds LIGHT with a K_{D} from about 1 nM to about 10 nM. In embodiments, the HVEM protein binds LIGHT with a K_{D} from about 1 nM to about 5 nM. In embodiments, the HVEM protein binds LIGHT with a K_{D} of about 1 nM, 5 nM, 10 nM, 15 nM, 20 nM, 25 nM, 30 nM, 35 nM, 40 nM, 45 nM, 50 nM, 55 nM, 60 nM, 65 nM, 70 nM, 75 nM, 80 nM, 85 nM, 90 nM, 95 nM or 100 nM.

In embodiments the HVEM protein binds LIGHT with a K_{D} from about 20 nM to about 40 nM. In embodiments the HVEM protein binds LIGHT with a K_{D} from about 22 nM to about 40 nM. In embodiments the HVEM protein binds LIGHT with a K_{D} from about 24 nM to about 40 nM. In embodiments the HVEM protein binds LIGHT with a K_{D} from about 26 nM to about 40 nM. In embodiments the HVEM protein binds LIGHT with a K_{D} from about 28 nM to about 40 nM. In embodiments the HVEM protein binds LIGHT with a K_{D} from about 30 nM to about 40 nM. In embodiments the HVEM protein binds LIGHT with a K_{D} from about 32 nM to about 40 nM. In embodiments the HVEM protein binds LIGHT with a K_{D} from about 34 nM to about 40 nM. In embodiments the HVEM protein binds LIGHT with a K_{D} from about 36 nM to about 40 nM. In embodiments the HVEM protein binds LIGHT with a K_{D} from about 38 nM to about 40 nM.

In embodiments the HVEM protein binds LIGHT with a K_{D} from about 20 nM to about 38 nM. In embodiments the HVEM protein binds LIGHT with a K_{D} from about 20 nM to about 36 nM. In embodiments the HVEM protein binds LIGHT with a K_{D} from about 20 nM to about 34 nM. In embodiments the HVEM protein binds LIGHT with a K_{D} from about 20 nM to about 32 nM. In embodiments the HVEM protein binds LIGHT with a K_{D} from about 20 nM to about 30 nM. In embodiments the HVEM protein binds LIGHT with a K_{D} from about 20 nM to about 28 nM. In embodiments the HVEM protein binds LIGHT with a K_{D} from about 20 nM to about 26 nM. In embodiments the HVEM protein binds LIGHT with a K_{D} from about 20 nM to about 24 nM. In embodiments the HVEM protein binds LIGHT with a K_{D} from about 20 nM to about 22 nM. In embodiments the HVEM protein binds LIGHT with a K_{D} of about 20 nM, 22 nM, 24 nM, 26 nM, 28 nM, 30 nM, 32 nM, 34 nM, 36 nM, 38 nM, or 40 nM. In embodiments, the HVEM protein binds LIGHT with a K_{D} of about 29.3 nM. In embodiments, the HVEM protein binds LIGHT with a K_{D} of 29.3 nM.

In embodiments, the HVEM protein binds BTLA with an equilibrium dissociation constant (K_{D}) from about 0.5 uM to about 50 uM. In embodiments, the HVEM protein binds BTLA with an equilibrium dissociation constant (K_{D}) from about 2 uM to about 50 uM. In embodiments, the HVEM protein binds BTLA with an equilibrium dissociation constant (K_{D}) from about 4 uM to about 50 uM. In embodiments, the HVEM protein binds BTLA with an equilibrium dissociation constant (K_{D}) from about 6 uM to about 50 uM. In embodiments, the HVEM protein binds BTLA with an equilibrium dissociation constant (K_{D}) from about 8 uM to about 50 uM. In embodiments, the HVEM protein binds BTLA with an equilibrium dissociation constant (K_{D}) from about 10 uM to about 50 uM. In embodiments, the HVEM protein binds BTLA with an equilibrium dissociation constant (K_{D}) from about 12 uM to about 50 uM. In embodiments, the HVEM protein binds BTLA with an equilibrium dissociation constant (K_{D}) from about 14 uM to about 50 uM. In embodiments, the HVEM protein binds BTLA with an equilibrium dissociation constant (K_{D}) from about 16 uM to about 50 uM. In embodiments, the HVEM protein binds BTLA with an equilibrium dissociation constant (K_{D}) from about 18 uM to about 50 uM. In embodiments, the HVEM protein binds BTLA with an equilibrium dissociation constant (K_{D}) from about 20 uM to about 50 uM. In embodiments, the HVEM protein binds BTLA with an equilibrium dissociation constant (K_{D}) from about 22 uM to about 50 uM. In embodiments, the HVEM protein binds BTLA with an equilibrium dissociation constant (K_{D}) from about 24 uM to about 50 uM. In embodiments, the HVEM protein binds BTLA with an equilibrium dissociation constant (K_{D}) from about 26 uM to about 50 uM. In embodiments, the HVEM protein binds BTLA with an equilibrium dissociation constant (K_{D}) from about 28 uM to about 50 uM. In embodiments, the HVEM protein binds BTLA with an equilibrium dissociation constant (K_{D}) from about 30 uM to about 50 uM. In embodiments, the HVEM protein binds BTLA with an equilibrium dissociation constant (K_{D}) from about 32 uM to about 50 uM. In embodiments, the HVEM protein binds BTLA with an equilibrium dissociation constant (K_{D}) from about 34 uM to about 50 uM. In embodiments, the HVEM protein binds BTLA with an equilibrium dissociation constant (K_{D}) from about 36 uM to about 50 uM. In embodiments, the HVEM protein binds BTLA with an equilibrium dissociation constant (K_{D}) from about 38 uM to about 50 uM. In embodiments, the HVEM protein binds BTLA with an equilibrium dissociation constant (K_{D}) from about 40 uM to about 50 uM. In embodiments, the HVEM protein binds BTLA with an equilibrium dissociation constant (K_{D}) from about 42 uM to about 50 uM. In embodiments, the HVEM protein binds BTLA with an equilibrium dissociation constant (K_{D}) from about 44 uM to about 50 uM. In embodiments, the HVEM protein binds BTLA with an equilibrium dissociation constant (K_{D}) from about 46 uM to about 50 uM. In embodiments, the HVEM protein binds BTLA with an equilibrium dissociation constant (K_{D}) from about 48 uM to about 50 uM.

In embodiments, the HVEM protein binds BTLA with an equilibrium dissociation constant (K_{D}) from about 0.5 uM to about 48 uM. In embodiments, the HVEM protein binds BTLA with an equilibrium dissociation constant (K_{D}) from about 0.5 uM to about 46 uM. In embodiments, the HVEM protein binds BTLA with an equilibrium dissociation constant (K_{D}) from about 0.5 uM to about 44 uM. In embodiments, the HVEM protein binds BTLA with an equilibrium dissociation constant (K_{D}) from about 0.5 uM to about 42 uM. In embodiments, the HVEM protein binds BTLA with an equilibrium dissociation constant (K_{D}) from about 0.5 uM to about 40 uM. In embodiments, the HVEM protein binds BTLA with an equilibrium dissociation constant (K_{D}) from about 0.5 uM to about 38 uM. In embodiments, the HVEM protein binds BTLA with an equilibrium dissociation constant (K_{D}) from about 0.5 uM to about 36 uM. In embodiments, the HVEM protein binds BTLA with an equilibrium dissociation constant (K_{D}) from about 0.5 uM to about 34 uM. In embodiments, the HVEM protein binds BTLA with an equilibrium dissociation constant (K_{D}) from about 0.5 uM to about 32 uM. In embodiments, the HVEM protein binds BTLA with an equilibrium dissociation constant (K_{D}) from about 0.5 uM to about 30 uM. In embodiments, the HVEM protein binds BTLA with an equilibrium dissociation constant (K_{D}) from about 0.5 uM to about 28 uM. In embodiments, the HVEM protein binds BTLA with an equilibrium dissociation constant (K_{D}) from about 0.5 uM to about 26 uM. In embodiments, the HVEM protein binds BTLA with an equilibrium dissociation constant (K_{D}) from about 0.5 uM to about 24 uM. In embodiments, the HVEM protein binds BTLA with an equilibrium dissociation constant (K_{D}) from about 0.5 uM to about 22 uM. In embodiments, the HVEM protein binds BTLA with an equilibrium dissociation constant (K_{D}) from about 0.5 uM to about 20 uM. In embodiments, the HVEM protein binds BTLA with an equilibrium dissociation constant (K_{D}) from about 0.5 uM to about 18 uM. In embodiments, the HVEM protein binds BTLA with an equilibrium dissociation constant (K_{D}) from about 0.5 uM to about 16 uM. In embodiments, the HVEM protein binds BTLA with an equilibrium dissociation constant (K_{D}) from about 0.5 uM to about 14 uM. In embodiments, the HVEM protein binds BTLA with an equilibrium dissociation constant (K_{D}) from about 0.5 uM to about 12 uM. In embodiments, the HVEM protein binds BTLA with an equilibrium dissociation constant (K_{D}) from about 0.5 uM to about 10 uM. In embodiments, the HVEM protein binds BTLA with an equilibrium dissociation constant (K_{D}) from about 0.5 uM to about 8 uM. In embodiments, the HVEM protein binds BTLA with an equilibrium dissociation constant (K_{D}) from about 0.5 uM to about 6 uM. In embodiments, the HVEM protein binds BTLA with an equilibrium dissociation constant (K_{D}) from about 0.5 uM to about 4 uM. In embodiments, the HVEM protein binds BTLA with an equilibrium dissociation constant (K_{D}) from about 0.5 uM to about 2 uM. In embodiments, the HVEM protein binds BTLA with an equilibrium dissociation constant (K_{D}) of about 0.5 uM, 2 uM, 4 uM, 6 uM, 8 uM, 10 uM, 12 uM, 14 uM, 16 uM, 18 uM, 20 uM, 22 uM, 24 uM, 26 uM, 28 uM, 30 uM, 32 uM, 34 uM, 36 uM, 38 uM, 40 uM, 42 uM, 44 uM, 46 uM, 48 uM, or 50 uM.

In embodiments, the HVEM protein binds BTLA with a K_{D} from about 2 uM to about 14 uM. In embodiments, the HVEM protein binds BTLA with a K_{D} from about 3 uM to about 14 uM. In embodiments, the HVEM protein binds BTLA with a K_{D} from about 4 uM to about 14 uM. In embodiments, the HVEM protein binds BTLA with a K_{D} from about 5 uM to about 14 uM. In embodiments, the HVEM protein binds BTLA with a K_{D} from about 6 uM to about 14 uM. In embodiments, the HVEM protein binds BTLA with a K_{D} from about 7 uM to about 14 uM. In embodiments, the HVEM protein binds BTLA with a K_{D} from about 8 uM to about 14 uM. In embodiments, the HVEM protein binds BTLA with a K_{D} from about 9 uM to about 14 uM. In embodiments, the HVEM protein binds BTLA with a K_{D} from about 10 uM to about 14 uM. In embodiments, the HVEM protein binds BTLA with a K_{D} from about 11 uM to about 14 uM. In embodiments, the HVEM protein binds BTLA with a K_{D} from about 12 uM to about 14 uM.

In embodiments, the HVEM protein binds BTLA with a K_{D} from about 2 uM to about 13 uM. In embodiments, the HVEM protein binds BTLA with a K_{D} from about 2 uM to about 12 uM. In embodiments, the HVEM protein binds BTLA with a K_{D} from about 2 uM to about 11 uM. In embodiments, the HVEM protein binds BTLA with a K_{D} from about 2 uM to about 10 uM. In embodiments, the HVEM protein binds BTLA with a K_{D} from about 2 uM to about 9 uM. In embodiments, the HVEM protein binds BTLA with a K_{D} from about 2 uM to about 8 uM. In embodiments, the HVEM protein binds BTLA with a K_{D} from about 2 uM to about 7 uM. In embodiments, the HVEM protein binds BTLA with a K_{D} from about 2 uM to about 6 uM. In embodiments, the HVEM protein binds BTLA with a K_{D} from about 2 uM to about 5 uM. In embodiments, the HVEM protein binds BTLA with a K_{D} from about 2 uM to about 4 uM. In embodiments, the HVEM protein binds BTLA with a K_{D} from about 2 uM to about 3 uM. In embodiments, the HVEM protein binds BTLA with a K_{D} of about 2 uM, 3 uM, 4 uM, 5 uM, 6 uM, 7 uM, 8 uM, 9 uM, 10 uM, 11 uM, 12 uM, 13 uM, 14 uM. In embodiments, the HVEM protein binds BTLA with a K_{D} of about 5.3 uM. n embodiments, the HVEM protein binds BTLA with a K_{D} of 5.3 uM.

In embodiments, the HVEM protein binds BTLA with a K_{D} from about 0.1 uM to about 4.1 uM. In embodiments, the HVEM protein binds BTLA with a K_{D} from about 0.5 uM to about 4.1 uM. In embodiments, the HVEM protein binds BTLA with a K_{D} from about 0.9 uM to about 4.1 uM. In embodiments, the HVEM protein binds BTLA with a K_{D} from about 1.3 uM to about 4.1 uM. In embodiments, the HVEM protein binds BTLA with a K_{D} from about 1.7 uM to about 4.1 uM. In embodiments, the HVEM protein binds BTLA with a K_{D} from about 2.1 uM to about 4.1 uM. In embodiments, the HVEM protein binds BTLA with a K_{D} from about 2.5 uM to about 4.1 uM. In embodiments, the HVEM protein binds BTLA with a K_{D} from about 2.9 uM to about 4.1 uM. In embodiments, the HVEM protein binds BTLA with a K_{D} from about 3.3 uM to about 4.1 uM. In embodiments, the HVEM protein binds BTLA with a K_{D} from about 3.7 uM to about 4.1 uM.

In embodiments, the HVEM protein binds BTLA with a K_{D} from about 0.1 uM to about 3.7 uM. In embodiments, the HVEM protein binds BTLA with a K_{D} from about 0.1 uM to about 3.3 uM. In embodiments, the HVEM protein binds BTLA with a K_{D} from about 0.1 uM to about 2.9 uM. In embodiments, the HVEM protein binds BTLA with a K_{D} from about 0.1 uM to about 2.5 uM. In embodiments, the HVEM protein binds BTLA with a K_{D} from about 0.1 uM to about 2.1 uM. In embodiments, the HVEM protein binds BTLA with a K_{D} from about 0.1 uM to about 1.7 uM. In embodiments, the HVEM protein binds BTLA with a K_{D} from about 0.1 uM to about 1.3 uM. In embodiments, the HVEM protein binds BTLA with a K_{D} from about 0.1 uM to about 0.9 uM. In embodiments, the HVEM protein binds BTLA with a K_{D} from about 0.1 uM to about 0.5 uM. In embodiments, the HVEM protein binds BTLA with a K_{D} from about 0.1 uM to about 0.1 uM. In embodiments, the HVEM protein binds BTLA with a K_{D} of about 0.1 uM, 0.5 uM, 0.9 uM. 1.3 uM, 1.7 uM, 2.1 uM, 2.5 uM, 2.9 uM, 3.3 uM, 3.7 uM or 4.1 uM. In embodiments, the HVEM protein binds BTLA with a K_{D} of about 1.1 uM. In embodiments, the HVEM protein binds BTLA with a K_{D} of 1.1 uM. In embodiments, the HVEM protein binds BTLA with a K_{D} of about 1 uM. In embodiments, the HVEM protein binds BTLA with a K_{D} of 1 uM. In embodiments, the HVEM protein binds BTLA with a K_{D} of about 0.9 uM. In embodiments, the HVEM protein binds BTLA with a K_{D} of 0.9 uM.

In embodiments, the HVEM protein binds CD160 with a K_{D} from about 10 uM to about 100 uM. In embodiments, the HVEM protein binds CD160 with a K_{D} from about 15 uM to about 100 uM. In embodiments, the HVEM protein binds CD160 with a K_{D} from about 20 uM to about 100 uM. In embodiments, the HVEM protein binds CD160 with a K_{D} from about 25 uM to about 100 uM. In embodiments, the HVEM protein binds CD160 with a K_{D} from about 30 uM to about 100 uM. In embodiments, the HVEM protein binds CD160 with a K_{D} from about 35 uM to about 100 uM. In embodiments, the HVEM protein binds CD160 with a K_{D} from about 40 uM to about 100 uM. In embodiments, the HVEM protein binds CD160 with a K_{D} from about 45 uM to about 100 uM. In embodiments, the HVEM protein binds CD160 with a K_{D} from about 50 uM to about 100 uM. In embodiments, the HVEM protein binds CD160 with a K_{D} from about 55 uM to about 100 uM. In embodiments, the HVEM protein binds CD160 with a K_{D} from about 60 uM to about 100 uM. In embodiments, the HVEM protein binds CD160 with a K_{D} from about 65 uM to about 100 uM. In embodiments, the HVEM protein binds CD160 with a K_{D} from about 70 uM to about 100 uM. In embodiments, the HVEM protein binds CD160 with a K_{D} from about 75 uM to about 100 uM. In embodiments, the HVEM protein binds CD160 with a K_{D} from about 80 uM to about 100 uM. In embodiments, the HVEM protein binds CD160 with a K_{D} from about 85 uM to about 100 uM. In embodiments, the HVEM protein binds CD160 with a K_{D} from about 90 uM to about 100 uM. In embodiments, the HVEM protein binds CD160 with a K_{D} from about 95 uM to about 100 uM.

In embodiments, the HVEM protein binds CD160 with a K_{D} from about 10 uM to about 95 uM. In embodiments, the HVEM protein binds CD160 with a K_{D} from about 10 uM to about 90 uM. In embodiments, the HVEM protein binds CD160 with a K_{D} from about 10 uM to about 85 uM. In embodiments, the HVEM protein binds CD160 with a K_{D} from about 10 uM to about 80 uM. In embodiments, the HVEM protein binds CD160 with a K_{D} from about 10 uM to about 75 uM. In embodiments, the HVEM protein binds CD160 with a K_{D} from about 10 uM to about 70 uM. In embodiments, the HVEM protein binds CD160 with a K_{D} from about 10 uM to about 65 uM. In embodiments, the HVEM protein binds CD160 with a K_{D} from about 10 uM to about 60 uM. In embodiments, the HVEM protein binds CD160 with a K_{D} from about 10 uM to about 55 uM. In embodiments, the HVEM protein binds CD160 with a K_{D} from about 10 uM to about 50 uM. In embodiments, the HVEM protein binds CD160 with a K_{D} from about 10 uM to about 45 uM. In embodiments, the HVEM protein binds CD160 with a K_{D} from about 10 uM to about 40 uM. In embodiments, the HVEM protein binds CD160 with a K_{D} from about 10 uM to about 35 uM. In embodiments, the HVEM protein binds CD160 with a K_{D} from about 10 uM to about 30 uM. In embodiments, the HVEM protein binds CD160 with a K_{D} from about 10 uM to about 25 uM. In embodiments, the HVEM protein binds CD160 with a K_{D} from about 10 uM to about 20 uM. In embodiments, the HVEM protein binds CD160 with a K_{D} from about 10 uM to about 15 uM. In embodiments, the HVEM protein binds CD160 with a K_{D} of about 10 uM, 15 uM, 20 uM, 25 uM, 30 uM, 35 uM, 40 uM, 45 uM, 50 uM, 55 uM, 60 uM, 65 uM, 70 uM, 75 uM, 80 uM, 85 uM, 90 uM, 95 uM, or 100 uM.

In embodiments, the HVEM protein binds CD160 with a K_{D} from about 14 uM to about 50 uM. In embodiments, the HVEM protein binds CD160 with a K_{D} from about 16 uM to about 50 uM. In embodiments, the HVEM protein binds CD160 with a K_{D} from about 18 uM to about 50 uM. In embodiments, the HVEM protein binds CD160 with a K_{D} from about 20 uM to about 50 uM. In embodiments, the HVEM protein binds CD160 with a K_{D} from about 22 uM to about 50 uM. In embodiments, the HVEM protein binds CD160 with a K_{D} from about 24 uM to about 50 uM. In embodiments, the HVEM protein binds CD160 with a K_{D} from about 26 uM to about 50 uM. In embodiments, the HVEM protein binds CD160 with a K_{D} from about 28 uM to about 50 uM. In embodiments, the HVEM protein binds CD160 with a K_{D} from about 30 uM to about 50 uM. In embodiments, the HVEM protein binds CD160 with a K_{D} from about 32 uM to about 50 uM. In embodiments, the HVEM protein binds CD160 with a K_{D} from about 34 uM to about 50 uM. In embodiments, the HVEM protein binds CD160 with a K_{D} from about 36 uM to about 50 uM. In embodiments, the HVEM protein binds CD160 with a K_{D} from about 38 uM to about 50 uM. In embodiments, the HVEM protein binds CD160 with a K_{D} from about 40 uM to about 50 uM. In embodiments, the HVEM protein binds CD160 with a K_{D} from about 42 uM to about 50 uM. In embodiments, the HVEM protein binds CD160 with a K_{D} from about 44 uM to about 50 uM. In embodiments, the HVEM protein binds CD160 with a K_{D} from about 46 uM to about 50 uM. In embodiments, the HVEM protein binds CD160 with a K_{D} from about 48 uM to about 50 uM.

In embodiments, the HVEM protein binds CD160 with a K_{D} from about 14 uM to about 48 uM. In embodiments, the HVEM protein binds CD160 with a K_{D} from about 14 uM to about 46 uM. In embodiments, the HVEM protein binds CD160 with a K_{D} from about 14 uM to about 44 uM. In embodiments, the HVEM protein binds CD160 with a K_{D} from about 14 uM to about 42 uM. In embodiments, the HVEM protein binds CD160 with a K_{D} from about 14 uM to about 40 uM. In embodiments, the HVEM protein binds CD160 with a K_{D} from about 14 uM to about 38 uM. In embodiments, the HVEM protein binds CD160 with a K_{D} from about 14 uM to about 36 uM. In embodiments, the HVEM protein binds CD160 with a K_{D} from about 14 uM to about 34 uM. In embodiments, the HVEM protein binds CD160 with a K_{D} from about 14 uM to about 32 uM. In embodiments, the HVEM protein binds CD160 with a K_{D} from about 14 uM to about 30 uM. In embodiments, the HVEM protein binds CD160 with a K_{D} from about 14 uM to about 28 uM. In embodiments, the HVEM protein binds CD160 with a K_{D} from about 14 uM to about 26 uM. In embodiments, the HVEM protein binds CD160 with a K_{D} from about 14 uM to about 24 uM. In embodiments, the HVEM protein binds CD160 with a K_{D} from about 14 uM to about 22 uM. In embodiments, the HVEM protein binds CD160 with a K_{D} from about 14 uM to about 20 uM. In embodiments, the HVEM protein binds CD160 with a K_{D} from about 14 uM to about 18 uM. In embodiments, the HVEM protein binds CD160 with a K_{D} from about 14 uM to about 16 uM. In embodiments, the HVEM protein binds CD160 with a K_{D} of about 14 uM, 16 uM, 18 uM, 20 uM, 22 uM, 24 uM, 26 uM, 28 uM, 30 uM, 32 uM, 34 uM, 36 uM, 38 uM, 40 uM, 42 uM, 44 uM, 46 uM, 48 uM, or 50 uM. In embodiments, the HVEM protein binds CD160 with a K_{D} of about 33.5 uM. In embodiments, the HVEM protein binds CD160 with a K_{D} of 33.5 uM.

In embodiments, the HVEM protein binds CD160 with a K_{D} from about 0.1 uM to about 4.1 uM. In embodiments, the HVEM protein binds CD160 with a K_{D} from about 0.5 uM to about 4.1 uM. In embodiments, the HVEM protein binds CD160 with a K_{D} from about 0.9 uM to about 4.1 uM. In embodiments, the HVEM protein binds CD160 with a K_{D} from about 1.3 uM to about 4.1 uM. In embodiments, the HVEM protein binds CD160 with a K_{D} from about 1.7 uM to about 4.1 uM. In embodiments, the HVEM protein binds CD160 with a K_{D} from about 2.1 uM to about 4.1 uM. In embodiments, the HVEM protein binds CD160 with a K_{D} from about 2.5 uM to about 4.1 uM. In embodiments, the HVEM protein binds CD160 with a K_{D} from about 2.9 uM to about 4.1 uM. In embodiments, the HVEM protein binds CD160 with a K_{D} from about 3.3 uM to about 4.1 uM. In embodiments, the HVEM protein binds CD160 with a K_{D} from about 3.7 uM to about 4.1 uM.

In embodiments, the HVEM protein binds CD160 with a K_{D} from about 0.1 uM to about 3.7 uM. In embodiments, the HVEM protein binds CD160 with a K_{D} from about 0.1 uM to about 3.3 uM. In embodiments, the HVEM protein binds CD160 with a K_{D} from about 0.1 uM to about 2.9 uM. In embodiments, the HVEM protein binds CD160 with a K_{D} from about. 1 uM to about 2.5 uM. In embodiments, the HVEM protein binds CD160 with a K_{D} from about 0.1 uM to about 2.1 uM. In embodiments, the HVEM protein binds CD160 with a K_{D} from about 0.1 uM to about 1.7 uM. In embodiments, the HVEM protein binds CD160 with a K_{D} from about 0.1 uM to about 1.3 uM. In embodiments, the HVEM protein binds CD160 with a K_{D} from about 0.1 uM to about 0.9 uM. In embodiments, the HVEM protein binds CD160 with a K_{D} from about 0.1 uM to about 0.5 uM. In embodiments, the HVEM protein binds CD160 with a K_{D} of about 0.1 uM, 0.5 uM, 0.9 uM. 1.3 uM, 1.7 uM, 2.1 uM, 2.5 uM, 2.9 uM, 3.3 uM, 3.7 uM or 4.1 uM. In embodiments, the HVEM protein binds CD160 with a K_{D} of about 1.3 uM. In embodiments, the HVEM protein binds CD160 with a K_{D} of 1.3 uM. In embodiments, the HVEM protein binds CD160 with a K_{D} of about 0.8 uM. In embodiments, the HVEM protein binds CD160 with a K_{D} of 0.8 uM. In embodiments, the HVEM protein binds CD160 with a K_{D} of about 0.9 uM. In embodiments, the HVEM protein binds CD160 with a K_{D} of 0.9 uM. In embodiments, the HVEM protein binds CD160 with a K_{D} of about 1.1 uM. In embodiments, the HVEM protein binds CD160 with a K_{D} of 1.1 uM.

TABLE 1
HVEM mutations and their effects

| Species | Mutation(s) | Mutation effect |
|---|---|---|
| Mouse | wild type (UniProt ID: | wild-type |
| | Q80WM9) | |
| Mouse | G72A | decreased CD160 binding |
| Mouse | G72D | decreased BTLA and CD160 |
| | | binding |
| Mouse | G72R | decreased BTLA and CD160 |
| | | binding |
| Mouse | T73A | decreased BTLA and CD160 |
| | | binding |
| Mouse | T73D | decreased BTLA and CD160 |
| | | binding |
| Mouse | V74D | decreased BTLA and CD160 |
| | | binding |
| Mouse | A76D | decreased CD160 binding |
| Mouse | T82D | decreased LIGHT binding |
| Mouse | Y83A | decreased LIGHT binding |
| Mouse | T84D | decreased LIGHT binding |
| Mouse | T84F | decreased LIGHT binding |
| Mouse | H86D | decreased LIGHT binding |
| Mouse | N88A | decreased LIGHT binding |
| Mouse | N88D | decreased LIGHT binding |
| Mouse | G89D | decreased LIGHT binding |
| Mouse | L90A | decreased LIGHT binding |
| Mouse | L90D | decreased LIGHT binding |
| Mouse | L90R | decreased LIGHT binding |
| Mouse | L94A | decreased LIGHT binding |
| Mouse | L94D | decreased LIGHT binding |
| Mouse | G72A-V74D | decreased BTLA and CD160 |
| | | binding |
| Mouse | G72D-V74A | decreased BTLA and CD160 |
| | | binding |
| Mouse | G72D-V74D | decreased BTLA and CD160 |
| | | binding |
| Mouse | G72D-V74R | decreased BTLA and CD160 |
| | | binding |
| Mouse | G72R-V74A | decreased BTLA and CD160 |
| | | binding |
| Mouse | H86A-L90A | decreased LIGHT binding |
| Mouse | H86D-L90A | decreased LIGHT binding |
| Mouse | H86D-L90D | decreased LIGHT binding |
| Mouse | H86D-L90R | decreased LIGHT binding |
| Mouse | H86R-L90A | decreased LIGHT binding |
| Mouse | A76D-H86D-L90A | decreased CD160 and LIGHT |
| | | binding |
| Mouse | A76D-H86D-L90D | decreased CD160 and LIGHT |
| | | binding |
| Human | Wild Type (UniProt ID: | wild-type |
| | Q92956) | |
| Human | G72 | decreased BTLA and CD160 |
| | | binding |
| Human | G72D | decreased BTLA and CD160 |
| | | binding |
| Human | T73 | decreased BTLA and CD160 |
| | | binding |
| Human | T73A | decreased BTLA and CD160 |
| | | binding |
| Human | V74 | decreased BTLA and CD160 |
| | | binding |
| Human | V74E | decreased BTLA and CD160 |
| | | binding |
| Human | E76 | decreased BTLA and CD160 |
| | | binding |
| Human | T82 | decreased LIGHT binding |
| Human | Y83 | decreased LIGHT binding |
| Human | I84 | decreased LIGHT binding |
| Human | H86 | decreased LIGHT binding |
| Human | N88 | decreased LIGHT binding |
| Human | N88A | decreased LIGHT binding |
| Human | G89 | decreased LIGHT binding |
| Human | L90 | decreased LIGHT binding |
| Human | L94 | decreased LIGHT binding |
| Human | G72-V74 | decreased BTLA and CD160 |
| | | binding |
| Human | H86-L90 | decreased LIGHT binding |

TABLE 2

| | | Mutation | | SEQ |
|---|---|---|---|---|
| Species | Mutation(s) | effect | Ectodomain protein sequences | ID NO |
| Mouse | wild type | wild-type | QPSCRQEEFLVGDECCPMCNPGYHV | 17 |
| | | | KQVCSEHTGTVCAPCPPQTYTAHAN | |
| | | | GLSKCLPCGVCDPDMGLLTWQECSS | |
| | | | WKDTVCRCIPGYFCENQDGSHCSTC | |
| | | | LQHTTCPPGQRVEKRGTHDQDTVC | |
| | | | ADCLTGTFSLGGTQEECLPWTNCSA | |
| | | | FQQEVRRGTNSTDTTCSSQVVYY | |
| Mouse | G72A | decreased | QPSCRQEEFLVGDECCPMCNPGYHV | 18 |
| | | CD160 | KQVCSEHTATVCAPCPPQTYTAHAN | |
| | | binding | GLSKCLPCGVCDPDMGLLTWQECSS | |
| | | | WKDTVCRCIPGYFCENQDGSHCSTC | |
| | | | LQHTTCPPGQRVEKRGTHDQDTVC | |
| | | | ADCLTGTFSLGGTQEECLPWTNCSA | |
| | | | FQQEVRRGTNSTDTTCSSQVVYY | |
| Mouse | G72D | decreased | QPSCRQEEFLVGDECCPMCNPGYHV | 19 |
| | | BTLA and | KQVCSEHTDTVCAPCPPQTYTAHAN | |
| | | CD160 | GLSKCLPCGVCDPDMGLLTWQECSS | |
| | | binding | WKDTVCRCIPGYFCENQDGSHCSTC | |
| | | | LQHTTCPPGQRVEKRGTHDQDTVC | |
| | | | ADCLTGTFSLGGTQEECLPWTNCSA | |
| | | | FQQEVRRGTNSTDTTCSSQVVYY | |
| Mouse | G72R | decreased | QPSCRQEEFLVGDECCPMCNPGYHV | 20 |
| | | BTLA and | KQVCSEHTRTVCAPCPPQTYTAHAN | |
| | | CD160 | GLSKCLPCGVCDPDMGLLTWQECSS | |
| | | binding | WKDTVCRCIPGYFCENQDGSHCSTC | |
| | | | LQHTTCPPGQRVEKRGTHDQDTVC | |
| | | | ADCLTGTFSLGGTQEECLPWTNCSA | |
| | | | FQQEVRRGTNSTDTTCSSQVVYY | |
| Mouse | T73A | decreased | QPSCRQEEFLVGDECCPMCNPGYHV | 21 |
| | | BTLA and | KQVCSEHTGAVCAPCPPQTYTAHA | |
| | | CD160 | NGLSKCLPCGVCDPDMGLLTWQEC | |
| | | binding | SSWKDTVCRCIPGYFCENQDGSHCS | |
| | | | TCLQHTTCPPGQRVEKRGTHDQDTV | |
| | | | CADCLTGTFSLGGTQEECLPWTNCS | |
| | | | AFQQEVRRGTNSTDTTCSSQVVYY | |
| Mouse | T73D | decreased | QPSCRQEEFLVGDECCPMCNPGYHV | 22 |
| | | BTLA and | KQVCSEHTGDVCAPCPPQTYTAHA | |
| | | CD160 | NGLSKCLPCGVCDPDMGLLTWQEC | |
| | | binding | SSWKDTVCRCIPGYFCENQDGSHCS | |
| | | | TCLQHTTCPPGQRVEKRGTHDQDTV | |
| | | | CADCLTGTFSLGGTQEECLPWTNCS | |
| | | | AFQQEVRRGTNSTDTTCSSQVVYY | |
| Mouse | V74D | decreased | QPSCRQEEFLVGDECCPMCNPGYHV | 23 |
| | | BTLA and | KQVCSEHTGTDCAPCPPQTYTAHAN | |
| | | CD160 | GLSKCLPCGVCDPDMGLLTWQECSS | |
| | | binding | WKDTVCRCIPGYFCENQDGSHCSTC | |
| | | | LQHTTCPPGQRVEKRGTHDQDTVC | |
| | | | ADCLTGTFSLGGTQEECLPWTNCSA | |
| | | | FQQEVRRGTNSTDTTCSSQVVYY | |
| Mouse | A76D | decreased | QPSCRQEEFLVGDECCPMCNPGYHV | 24 |
| | | CD160 | KQVCSEHTGTVCDPCPPQTYTAHAN | |
| | | binding | GLSKCLPCGVCDPDMGLLTWQECSS | |
| | | | WKDTVCRCIPGYFCENQDGSHCSTC | |
| | | | LQHTTCPPGQRVEKRGTHDQDTVC | |
| | | | ADCLTGTFSLGGTQEECLPWTNCSA | |
| | | | FQQEVRRGTNSTDTTCSSQVVYY | |
| Mouse | T82D | decreased | QPSCRQEEFLVGDECCPMCNPGYHV | 25 |
| | | LIGHT | KQVCSEHTGTVCAPCPPQDYTAHA | |
| | | binding | NGLSKCLPCGVCDPDMGLLTWQEC | |
| | | | SSWKDTVCRCIPGYFCENQDGSHCS | |
| | | | TCLQHTTCPPGQRVEKRGTHDQDTV | |
| | | | CADCLTGTFSLGGTQEECLPWTNCS | |
| | | | AFQQEVRRGTNSTDTTCSSQVVYY | |
| Mouse | Y83A | decreased | QPSCRQEEFLVGDECCPMCNPGYHV | 26 |
| | | LIGHT | KQVCSEHTGTVCAPCPPQTATAHAN | |
| | | binding | GLSKCLPCGVCDPDMGLLTWQECSS | |
| | | | WKDTVCRCIPGYFCENQDGSHCSTC | |
| | | | LQHTTCPPGQRVEKRGTHDQDTVC | |
| | | | ADCLTGTFSLGGTQEECLPWTNCSA | |
| | | | FQQEVRRGTNSTDTTCSSQVVYY | |
| Mouse | T84D | decreased | QPSCRQEEFLVGDECCPMCNPGYHV | 27 |
| | | LIGHT | KQVCSEHTGTVCAPCPPQTYDAHA | |
| | | binding | NGLSKCLPCGVCDPDMGLLTWQEC | |
| | | | SSWKDTVCRCIPGYFCENQDGSHCS | |
| | | | TCLQHTTCPPGQRVEKRGTHDQDTV | |
| | | | CADCLTGTFSLGGTQEECLPWTNCS | |
| | | | AFQQEVRRGTNSTDTTCSSQVVYY | |
| Mouse | T84F | decreased | QPSCRQEEFLVGDECCPMCNPGYHV | 28 |
| | | LIGHT | KQVCSEHTGTVCAPCPPQTYFAHAN | |
| | | binding | GLSKCLPCGVCDPDMGLLTWQECSS | |
| | | | WKDTVCRCIPGYFCENQDGSHCSTC | |
| | | | LQHTTCPPGQRVEKRGTHDQDTVC | |
| | | | ADCLTGTFSLGGTQEECLPWTNCSA | |
| | | | FQQEVRRGTNSTDTTCSSQVVYY | |
| Mouse | H86D | decreased | QPSCRQEEFLVGDECCPMCNPGYHV | 29 |
| | | LIGHT | KQVCSEHTGTVCAPCPPQTYTADAN | |
| | | binding | GLSKCLPCGVCDPDMGLLTWQECSS | |
| | | | WKDTVCRCIPGYFCENQDGSHCSTC | |
| | | | LQHTTCPPGQRVEKRGTHDQDTVC | |
| | | | ADCLTGTFSLGGTQEECLPWTNCSA | |
| | | | FQQEVRRGTNSTDTTCSSQVVYY | |
| Mouse | N88A | decreased | QPSCRQEEFLVGDECCPMCNPGYHV | 30 |
| | | LIGHT | KQVCSEHTGTVCAPCPPQTYTAHAA | |
| | | binding | GLSKCLPCGVCDPDMGLLTWQECSS | |
| | | | WKDTVCRCIPGYFCENQDGSHCSTC | |
| | | | LQHTTCPPGQRVEKRGTHDQDTVC | |
| | | | ADCLTGTFSLGGTQEECLPWTNCSA | |
| | | | FQQEVRRGTNSTDTTCSSQVVYY | |
| Mouse | N88D | decreased | QPSCRQEEFLVGDECCPMCNPGYHV | 31 |
| | | LIGHT | KQVCSEHTGTVCAPCPPQTYTAHAD | |
| | | binding | GLSKCLPCGVCDPDMGLLTWQECSS | |
| | | | WKDTVCRCIPGYFCENQDGSHCSTC | |
| | | | LQHTTCPPGQRVEKRGTHDQDTVC | |
| | | | ADCLTGTFSLGGTQEECLPWTNCSA | |
| | | | FQQEVRRGTNSTDTTCSSQVVYY | |
| Mouse | G89D | decreased | QPSCRQEEFLVGDECCPMCNPGYHV | 32 |
| | | LIGHT | KQVCSEHTGTVCAPCPPQTYTAHAN | |
| | | binding | DLSKCLPCGVCDPDMGLLTWQECSS | |
| | | | WKDTVCRCIPGYFCENQDGSHCSTC | |
| | | | LQHTTCPPGQRVEKRGTHDQDTVC | |
| | | | ADCLTGTFSLGGTQEECLPWTNCSA | |
| | | | FQQEVRRGTNSTDTTCSSQVVYY | |
| Mouse | L90A | decreased | QPSCRQEEFLVGDECCPMCNPGYHV | 33 |
| | | LIGHT | KQVCSEHTGTVCAPCPPQTYTAHAN | |
| | | binding | GASKCLPCGVCDPDMGLLTWQECS | |
| | | | SWKDTVCRCIPGYFCENQDGSHCST | |
| | | | CLQHTTCPPGQRVEKRGTHDQDTV | |
| | | | CADCLTGTFSLGGTQEECLPWTNCS | |
| | | | AFQQEVRRGTNSTDTTCSSQVVYY | |
| Mouse | L90D | decreased | QPSCRQEEFLVGDECCPMCNPGYHV | 34 |
| | | LIGHT | KQVCSEHTGTVCAPCPPQTYTAHAN | |
| | | binding | GDSKCLPCGVCDPDMGLLTWQECS | |
| | | | SWKDTVCRCIPGYFCENQDGSHCST | |
| | | | CLQHTTCPPGQRVEKRGTHDQDTV | |
| | | | CADCLTGTFSLGGTQEECLPWTNCS | |
| | | | AFQQEVRRGTNSTDTTCSSQVVYY | |
| Mouse | L90R | decreased | QPSCRQEEFLVGDECCPMCNPGYHV | 35 |
| | | LIGHT | KQVCSEHTGTVCAPCPPQTYTAHAN | |
| | | binding | GRSKCLPCGVCDPDMGLLTWQECS | |
| | | | SWKDTVCRCIPGYFCENQDGSHCST | |
| | | | CLQHTTCPPGQRVEKRGTHDQDTV | |
| | | | CADCLTGTFSLGGTQEECLPWTNCS | |
| | | | AFQQEVRRGTNSTDTTCSSQVVYY | |
| Mouse | L94A | decreased | QPSCRQEEFLVGDECCPMCNPGYHV | 36 |
| | | LIGHT | KQVCSEHTGTVCAPCPPQTYTAHAN | |
| | | binding | GLSKCAPCGVCDPDMGLLTWQECS | |
| | | | SWKDTVCRCIPGYFCENQDGSHCST | |
| | | | CLQHTTCPPGQRVEKRGTHDQDTV | |
| | | | CADCLTGTFSLGGTQEECLPWTNCS | |
| | | | AFQQEVRRGTNSTDTTCSSQVVYY | |
| Mouse | L94D | decreased | QPSCRQEEFLVGDECCPMCNPGYHV | 37 |
| | | LIGHT | KQVCSEHTGTVCAPCPPQTYTAHAN | |
| | | binding | GLSKCDPCGVCDPDMGLLTWQECS | |
| | | | SWKDTVCRCIPGYFCENQDGSHCST | |
| | | | CLQHTTCPPGQRVEKRGTHDQDTV | |
| | | | CADCLTGTFSLGGTQEECLPWTNCS | |
| | | | AFQQEVRRGTNSTDTTCSSQVVYY | |
| Mouse | G72A- | decreased | QPSCRQEEFLVGDECCPMCNPGYHV | 38 |
| | V74D | BTLA and | KQVCSEHTATDCAPCPPQTYTAHAN | |
| | | CD160 | GLSKCLPCGVCDPDMGLLTWQECSS | |
| | | binding | WKDTVCRCIPGYFCENQDGSHCSTC | |
| | | | LQHTTCPPGQRVEKRGTHDQDTVC | |
| | | | ADCLTGTFSLGGTQEECLPWTNCSA | |
| | | | FQQEVRRGTNSTDTTCSSQVVYY | |
| Mouse | G72D- | decreased | QPSCRQEEFLVGDECCPMCNPGYHV | 39 |
| | V74A | BTLA and | KQVCSEHTDTACAPCPPQTYTAHAN | |
| | | CD160 | GLSKCLPCGVCDPDMGLLTWQECSS | |
| | | binding | WKDTVCRCIPGYFCENQDGSHCSTC | |
| | | | LQHTTCPPGQRVEKRGTHDQDTVC | |
| | | | ADCLTGTFSLGGTQEECLPWTNCSA | |
| | | | FQQEVRRGTNSTDTTCSSQVVYY | |
| Mouse | G72D- | decreased | QPSCRQEEFLVGDECCPMCNPGYHV | 40 |
| | V74D | BTLA and | KQVCSEHTDTDCAPCPPQTYTAHAN | |
| | | CD160 | GLSKCLPCGVCDPDMGLLTWQECSS | |
| | | binding | WKDTVCRCIPGYFCENQDGSHCSTC | |
| | | | LQHTTCPPGQRVEKRGTHDQDTVC | |
| | | | ADCLTGTFSLGGTQEECLPWTNCSA | |
| | | | FQQEVRRGTNSTDTTCSSQVVYY | |
| Mouse | G72D- | decreased | QPSCRQEEFLVGDECCPMCNPGYHV | 41 |
| | V74R | BTLA and | KQVCSEHTDTRCAPCPPQTYTAHAN | |
| | | CD160 | GLSKCLPCGVCDPDMGLLTWQECSS | |
| | | binding | WKDTVCRCIPGYFCENQDGSHCSTC | |
| | | | LQHTTCPPGQRVEKRGTHDQDTVC | |
| | | | ADCLTGTFSLGGTQEECLPWTNCSA | |
| | | | FQQEVRRGTNSTDTTCSSQVVYY | |
| Mouse | G72R- | decreased | QPSCRQEEFLVGDECCPMCNPGYHV | 42 |
| | V74A | BTLA and | KQVCSEHTRTACAPCPPQTYTAHAN | |
| | | CD160 | GLSKCLPCGVCDPDMGLLTWQECSS | |
| | | binding | WKDTVCRCIPGYFCENQDGSHCSTC | |
| | | | LQHTTCPPGQRVEKRGTHDQDTVC | |
| | | | ADCLTGTFSLGGTQEECLPWTNCSA | |
| | | | FQQEVRRGTNSTDTTCSSQVVYY | |
| Mouse | H86A- | decreased | QPSCRQEEFLVGDECCPMCNPGYHV | 43 |
| | L90A | LIGHT | KQVCSEHTGTVCAPCPPQTYTAAAN | |
| | | binding | GASKCLPCGVCDPDMGLLTWQECS | |
| | | | SWKDTVCRCIPGYFCENQDGSHCST | |
| | | | CLQHTTCPPGQRVEKRGTHDQDTV | |
| | | | CADCLTGTFSLGGTQEECLPWTNCS | |
| | | | AFQQEVRRGTNSTDTTCSSQVVYY | |
| Mouse | H86D- | decreased | QPSCRQEEFLVGDECCPMCNPGYHV | 44 |
| | L90A | LIGHT | KQVCSEHTGTVCAPCPPQTYTADAN | |
| | | binding | GASKCLPCGVCDPDMGLLTWQECS | |
| | | | SWKDTVCRCIPGYFCENQDGSHCST | |
| | | | CLQHTTCPPGQRVEKRGTHDQDTV | |
| | | | CADCLTGTFSLGGTQEECLPWTNCS | |
| | | | AFQQEVRRGTNSTDTTCSSQVVYY | |
| Mouse | H86D- | decreased | QPSCRQEEFLVGDECCPMCNPGYHV | 45 |
| | L90D | LIGHT | KQVCSEHTGTVCAPCPPQTYTADAN | |
| | | binding | GDSKCLPCGVCDPDMGLLTWQECS | |
| | | | SWKDTVCRCIPGYFCENQDGSHCST | |
| | | | CLQHTTCPPGQRVEKRGTHDQDTV | |
| | | | CADCLTGTFSLGGTQEECLPWTNCS | |
| | | | AFQQEVRRGTNSTDTTCSSQVVYY | |
| Mouse | H86D-L90R | decreased | QPSCRQEEFLVGDECCPMCNPGYHV | 46 |
| | | LIGHT | KQVCSEHTGTVCAPCPPQTYTADAN | |
| | | binding | GRSKCLPCGVCDPDMGLLTWQECS | |
| | | | SWKDTVCRCIPGYFCENQDGSHCST | |
| | | | CLQHTTCPPGQRVEKRGTHDQDTV | |
| | | | CADCLTGTFSLGGTQEECLPWTNCS | |
| | | | AFQQEVRRGTNSTDTTCSSQVVYY | |
| Mouse | H86R-L90A | decreased | QPSCRQEEFLVGDECCPMCNPGYHV | 47 |
| | | LIGHT | KQVCSEHTGTVCAPCPPQTYTARAN | |
| | | binding | GASKCLPCGVCDPDMGLLTWQECS | |
| | | | SWKDTVCRCIPGYFCENQDGSHCST | |
| | | | CLQHTTCPPGQRVEKRGTHDQDTV | |
| | | | CADCLTGTFSLGGTQEECLPWTNCS | |
| | | | AFQQEVRRGTNSTDTTCSSQVVYY | |
| Mouse | A76D- | decreased | QPSCRQEEFLVGDECCPMCNPGYHV | 48 |
| | H86D- | CD160 | KQVCSEHTGTVCDPCPPQTYTADAN | |
| | L90A | and | GASKCLPCGVCDPDMGLLTWQECS | |
| | | LIGHT | SWKDTVCRCIPGYFCENQDGSHCST | |
| | | binding | CLQHTTCPPGQRVEKRGTHDQDTV | |
| | | | CADCLTGTFSLGGTQEECLPWTNCS | |
| | | | AFQQEVRRGTNSTDTTCSSQVVYY | |
| Mouse | A76D- | decreased | QPSCRQEEFLVGDECCPMCNPGYHV | 49 |
| | H86D- | CD160 | KQVCSEHTGTVCDPCPPQTYTADAN | |
| | L90D | and | GDSKCLPCGVCDPDMGLLTWQECS | |
| | | LIGHT | SWKDTVCRCIPGYFCENQDGSHCST | |
| | | binding | CLQHTTCPPGQRVEKRGTHDQDTV | |
| | | | CADCLTGTFSLGGTQEECLPWTNCS | |
| | | | AFQQEVRRGTNSTDTTCSSQVVYY | |
| Human | wild type | wild-type | LPSCKEDEYPVGSECCPKCSPGYRV | 50 |
| | | | KEACGELTGTVCEPCPPGTYIAHLN | |
| | | | GLSKCLQCQMCDPAMGLRASRNCS | |
| | | | RTENAVCGCSPGHFCIVQDGDHCAA | |
| | | | CRAYATSSPGQRVQKGGTESQDTLC | |
| | | | QNCPPGTFSPNGTLEECQHQTKCSW | |
| | | | LVTKAGAGTSSSHWV | |
| Human | G72D | decreased | LPSCKEDEYPVGSECCPKCSPGYRV | 51 |
| | | BTLA and | KEACGELTDTVCEPCPPGTYIAHLN | |
| | | CD160 | GLSKCLQCQMCDPAMGLRASRNCS | |
| | | binding | RTENAVCGCSPGHFCIVQDGDHCAA | |
| | | | CRAYATSSPGQRVQKGGTESQDTLC | |
| | | | QNCPPGTFSPNGTLEECQHQTKCSW | |
| | | | LVTKAGAGTSSSHWV | |
| Human | T73A | decreased | LPSCKEDEYPVGSECCPKCSPGYRV | 52 |
| | | BTLA and | KEACGELTGAVCEPCPPGTYIAHLN | |
| | | CD160 | GLSKCLQCQMCDPAMGLRASRNCS | |
| | | binding | RTENAVCGCSPGHFCIVQDGDHCAA | |
| | | | CRAYATSSPGQRVQKGGTESQDTLC | |
| | | | QNCPPGTFSPNGTLEECQHQTKCSW | |
| | | | LVTKAGAGTSSSHWV | |
| Human | V74E | decreased | LPSCKEDEYPVGSECCPKCSPGYRV | 53 |
| | | BTLA and | KEACGELTGTECEPCPPGTYIAHLNG | |
| | | CD160 | LSKCLQCQMCDPAMGLRASRNCSR | |
| | | binding | TENAVCGCSPGHFCIVQDGDHCAAC | |
| | | | RAYATSSPGQRVQKGGTESQDTLCQ | |
| | | | NCPPGTFSPNGTLEECQHQTKCSWL | |
| | | | VTKAGAGTSSSHWV | |
| Human | N88A | decreased | LPSCKEDEYPVGSECCPKCSPGYRV | 54 |
| | | LIGHT | KEACGELTGTVCEPCPPGTYIAHLA | |
| | | binding | GLSKCLQCQMCDPAMGLRASRNCS | |
| | | | RTENAVCGCSPGHFCIVQDGDHCAA | |
| | | | CRAYATSSPGQRVQKGGTESQDTLC | |
| | | | QNCPPGTFSPNGTLEECQHQTKCSW | |
| | | | LVTKAGAGTSSSHWV | |

### Chimeric Antigen Receptor Proteins

The compositions provided herein include chimeric antigen receptors including the HVEM protein provided herein or a fragment thereof. Thus, in an aspect is provided a Chimeric Antigen Receptor (CAR) including: a) an ectodomain of the HVEM protein provided herein including embodiments thereof, and b) a transmembrane domain.

An “ectodomain” as provided herein refers to the portion of a protein that is on the extracellular space (e.g. space outside) of the cell. For example, an ectodomain of the protein may bind to a receptor or a ligand (e.g. LIGHT, BTLA, CD160, etc.), thereby leading to signal transduction. An ectodomain may bind to a receptor or ligand expressed on the surface of another cell. In embodiments, a protein ectodomain may include one or more amino acid residues that is in contact with or is embedded in the membrane of a cell.

A “transmembrane domain” as provided herein refers to a polypeptide forming part of a biological membrane. The transmembrane domain provided herein is capable of spanning a biological membrane (e.g., a cellular membrane) from one side of the membrane through to the other side of the membrane. In embodiments, the transmembrane domain spans from the intracellular side to the extracellular side of a cellular membrane. Transmembrane domains may include non-polar, hydrophobic residues, which anchor the proteins provided herein including embodiments thereof in a biological membrane (e.g., cellular membrane of a T cell). Any transmembrane domain capable of anchoring the proteins provided herein including embodiments thereof are contemplated. Non-limiting examples of transmembrane domains include the transmembrane domains of CD28, CD8, CD4 or CD3-zeta. In embodiments, the transmembrane domain is a CD4 transmembrane domain.

In embodiments, the transmembrane domain is a CD28 transmembrane domain. The term “CD28 transmembrane domain” as provided herein includes any of the recombinant or naturally-occurring forms of the transmembrane domain of CD28, or variants or homologs thereof that maintain CD28 transmembrane domain activity (e.g. within at least 50%, 80%, 90%, 95%, 96%, 97%, 98%, 99% or 100% activity compared to the CD28 transmembrane domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD28 transmembrane domain polypeptide. In embodiments, CD28 is the protein as identified by the NCBI sequence reference GI: 340545506, homolog or functional fragment thereof.

In embodiments, the transmembrane domain is a CD8 transmembrane domain. The term “CD8 transmembrane domain” as provided herein includes any of the recombinant or naturally-occurring forms of the transmembrane domain of CD8, or variants or homologs thereof that maintain CD8 transmembrane domain activity (e.g. within at least 50%, 80%, 90%, 95%, 96%, 97%, 98%, 99% or 100% activity compared to the CD8 transmembrane domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD8 transmembrane domain polypeptide. In embodiments, CD8 is the protein as identified by the NCBI sequence reference GI: 225007534, homolog or functional fragment thereof.

In embodiments, the transmembrane domain is a CD4 transmembrane domain. The term “CD4 transmembrane domain” as provided herein includes any of the recombinant or naturally-occurring forms of the transmembrane domain of CD4, or variants or homologs thereof that maintain CD4 transmembrane domain activity (e.g. within at least 50%, 80%, 90%, 95%, 96%, 97%, 98%, 99% or 100% activity compared to the CD4 transmembrane domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD4 transmembrane domain polypeptide. In embodiments, CD4 is the protein as identified by the NCBI sequence reference GI: 303522473, homolog or functional fragment thereof.

In embodiments, the transmembrane domain is a CD3-zeta (also known as CD247) transmembrane domain. The term “CD3-zeta transmembrane domain” as provided herein includes any of the recombinant or naturally-occurring forms of the transmembrane domain of CD3-zeta, or variants or homologs thereof that maintain CD3-zeta transmembrane domain activity (e.g. within at least 50%, 80%, 90%, 95%, 96%, 97%, 98%, 99% or 100% activity compared to the CD3-zeta transmembrane domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD3-zeta transmembrane domain polypeptide. In embodiments, CD3-zeta is the protein as identified by the NCBI sequence reference GI: 166362721, homolog or functional fragment thereof.

In embodiments, the chimeric antigen receptor further includes an intracellular T-cell signaling domain. An “intracellular T-cell signaling domain” as provided herein includes amino acid sequences capable of providing primary signaling in response to binding of an antigen to the antibody region provided herein including embodiments thereof. In embodiments, the signaling of the intracellular T-cell signaling domain results in activation of the T cell expressing the same. In embodiments, the signaling of the intracellular T-cell signaling domain results in proliferation (cell division) of the T cell expressing the same. In embodiments, the signaling of the intracellular T-cell signaling domain results expression by said T cell of proteins known in the art to characteristic of activated T cell (e.g., CTLA-4, PD-1, CD28, CD69). In embodiments, the intracellular T-cell signaling domain is a CD3% intracellular T-cell signaling domain.

In embodiments, the chimeric antigen receptor further includes an intracellular co-stimulatory T-cell signaling domain. An “intracellular co-stimulatory signaling domain” as provided herein includes amino acid sequences capable of providing co-stimulatory signaling in response to binding of an antigen to the antibody region provided herein including embodiments thereof. In embodiments, the signaling of the co-stimulatory signaling domain results in production of cytokines and proliferation of the T cell expressing the same. In embodiments, the intracellular co-stimulatory signaling domain is a CD28 intracellular co-stimulatory signaling domain, a 4-1BB intracellular co-stimulatory signaling domain, an ICOS intracellular co-stimulatory signaling domain, or an OX-40 intracellular co-stimulatory signaling domain. In embodiments, the intracellular co-stimulatory signaling domain is a CD28 intracellular co-stimulatory signaling domain. In embodiments, the intracellular co-stimulatory signaling domain is a 4-1BB intracellular co-stimulatory signaling domain. In embodiments, the intracellular co-stimulatory signaling domain is an ICOS intracellular co-stimulatory signaling domain. In embodiments, the intracellular co-stimulatory signaling domain is an OX-40 intracellular co-stimulatory signaling domain.

The term “CTLA-4” as referred to herein includes any of the recombinant or naturally-occurring forms of the cytotoxic T-lymphocyte-associated protein 4 protein, also known as CD152 (cluster of differentiation 152), or variants or homologs thereof that maintain CTLA-4 activity (e.g. within at least 50%, 80%, 90%, 95%, 96%, 97%, 98%, 99% or 100% activity compared to CTLA-4). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CTLA-4 protein. In embodiments, the CTLA-4 protein is substantially identical to the protein identified by the UniProt reference number P16410 or a variant or homolog having substantial identity thereto.

The term “CD28” as referred to herein includes any of the recombinant or naturally-occurring forms of the Cluster of Differentiation 28 protein, or variants or homologs thereof that maintain CD28 activity (e.g. within at least 50%, 80%, 90%, 95%, 96%, 97%, 98%, 99% or 100% activity compared to CD28). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD28 protein. In embodiments, the CD28 protein is substantially identical to the protein identified by the UniProt reference number P10747 or a variant or homolog having substantial identity thereto.

The term “CD69” as referred to herein includes any of the recombinant or naturally-occurring forms of the Cluster of Differentiation 69 protein, or variants or homologs thereof that maintain CD69 activity (e.g. within at least 50%, 80%, 90%, 95%, 96%, 97%, 98%, 99% or 100% activity compared to CD69). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD69 protein. In embodiments, the CD69 protein is substantially identical to the protein identified by the UniProt reference number Q07108 or a variant or homolog having substantial identity thereto.

The term “4-1BB” as referred to herein includes any of the recombinant or naturally-occurring forms of the 4-1BB protein, also known as tumor necrosis factor receptor superfamily member 9 (TNFRSF9), Cluster of Differentiation 137 (CD137) and induced by lymphocyte activation (ILA), or variants or homologs thereof that maintain 4-1BB activity (e.g. within at least 50%, 80%, 90%, 95%, 96%, 97%, 98%, 99% or 100% activity compared to 4-1BB). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring EGFR protein. In embodiments, the 4-1BB protein is substantially identical to the protein identified by the UniProt reference number Q07011 or a variant or homolog having substantial identity thereto.

### Nucleic Acid Compositions

Compositions provided herein include nucleic acids encoding the HVEM protein provided herein or the chimeric antigen receptor provided herein. In an aspect is provided a nucleic acid encoding a HVEM protein provided herein including embodiments thereof. In another aspect is provided a nucleic acid encoding a CAR provided herein including embodiments thereof. The nucleic acid provided herein, including embodiments thereof, may be loaded into an expression vector such that the nucleic acid may be delivered to cells.

As used herein, the term “vector” refers to a nucleic acid molecule capable of transporting another nucleic acid to which it has been linked. One type of vector is a “plasmid”, which refers to a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. The terms “plasmid” and “vector” can be used interchangeably as the plasmid is the most commonly used form of vector. However, the disclosure is intended to include such other forms of expression vectors, such as viral vectors (e.g., replication defective retroviruses, adenoviruses and adeno-associated viruses), which serve equivalent functions. Some viral vectors are capable of targeting a particular cells type either specifically or non-specifically.

Thus, in embodiments, the nucleic acid further includes an expression vector. Thus, in an aspect, an expression vector including the nucleic acid provided herein, including embodiments thereof, is provided.

It is contemplated that the nucleic acid may be loaded into any expression vector useful for delivering the nucleic acid to cells either in vivo or in vitro. It is further contemplated that viruses, for example, lentivirus and onco-retrovirus, may serve as suitable expression vectors. Accordingly, in embodiments, the expression vector is a viral vector. In embodiments, the viral vector is a lentiviral vector or an onco-retroviral vector. In embodiments, the viral vector is a lentiviral vector. In embodiments, the viral vector is an onco-retroviral vector. In embodiments, the virus is a lentivirus or an onco-retrovirus. In embodiments, the virus is a lentivirus. In embodiments, the virus is an onco-retrovirus.

In embodiments, the viral vector is a replication-incompetent viral vector. In embodiments, the replication-incompetent viral vector is a replication-incompetent DNA viral vector (e.g. adenoviruses, adeno-associated viruses, etc.). In embodiments, the replication-incompetent viral vector is a replication-incompetent RNA viral vector (e.g. replication defective retroviruses, lentiviruses, rabies viruses, etc.).

### Cellular Compositions

Provided herein are cells including the compositions provided herein including embodiments thereof. Thus, in an aspect is provided a T cell including the Chimeric Antigen Receptor (CAR) provided herein including embodiments thereof.

In another aspect is provided a cell including the HVEM protein provided herein including embodiments thereof, or the nucleic acid provided herein including embodiments thereof. In embodiments, the cell includes the HVEM protein provided herein including embodiments thereof. In embodiments, the HVEM protein is a recombinant protein. In embodiments, the HVEM protein is heterologous to the cell. In embodiments, the cell includes the nucleic acid provided herein including embodiments thereof. In embodiments, the nucleic acid is heterologous to the cell.

### Methods of Treatment

The HVEM proteins provided herein including embodiments thereof are capable of selective interactions with ligands (e.g. LIGHT, BTLA, CD160). In embodiments, selective interaction with one or more ligands includes modulated binding affinity of an HVEM protein to one or more ligands. For example, HVEM proteins provided herein may have decreased binding affinity to LIGHT protein compared to the wild type HVEM protein. For example, HVEM proteins provided herein may have decreased binding affinity to BTLA and/or CD160 compared to the wild type HVEM protein. In embodiments, the HVEM protein having decreased binding affinity to LIGHT selectively binds to BTLA and/or CD160. In embodiments, the HVEM protein having decreased binding affinity to BTLA and/or CD160 selectively binds to LIGHT. Without wishing to be bound by scientific theory, the HVEM proteins through selective ligand interactions exert therapeutic effects including prevention of infection, inflammation, and cancer development. For example, HVEM proteins provided herein that selectively bind to BTLA are contemplated to be effective for treating and/or preventing BTLA-expressing cancers, including lymphoma. HVEM proteins provided herein that selectively bind BTLA may further be effective for treating autoimmune disease. In another example, HVEM proteins provided herein that have decreased binding affinity to LIGHT are contemplated to be effective for treating and/or preventing of cancer, viral infection, and autoimmune disorders. In another example, HVEM proteins provided herein that have decreased binding affinity to BTLA/CD1160 are contemplated to be effective for the treatment and/or prevention of bacterial infection (e.g. Yersinia enterocolitica, etc.). The compositions provided herein are therefore are contemplated as providing effective treatments for diseases, including cancer and autoimmune disorders. Thus, in an aspect is provided a method of treating or preventing a disease in a subject in need thereof, the method including administering to the subject an effective amount of a HVEM protein or fragment thereof provided herein including embodiments thereof, a nucleic acid provided herein including embodiments thereof, a CAR provided herein including embodiments thereof, or a T-cell provided herein including embodiments thereof. In embodiments, the method includes administering to the subject an effective amount of a HVEM protein or fragment thereof provided herein including embodiments thereof. In embodiments, the method includes administering to the subject an effective amount of a nucleic acid provided herein including embodiments thereof. In embodiments, the method includes administering to the subject an effective amount of a CAR provided herein including embodiments thereof. In embodiments, the method includes administering to the subject an effective amount of a T-cell provided herein including embodiments thereof.

In embodiments, the disease is cancer. In embodiments, the cancer is a liquid cancer. The term “liquid cancer” is used in accordance to its ordinary meaning in the art and refers to a cancer (e.g. leukemia, lymphoma) that originates from myeloid or lymphoid cells. In embodiments, the cancer is B cell lymphoma. In embodiments, the cancer is breast cancer, lung cancer, renal cancer, colorectal cancer, melanoma or bladder cancer. In embodiments, the cancer is breast cancer. In embodiments, the cancer is lung cancer. In embodiments, the cancer is renal cancer. In embodiments, the cancer is colorectal cancer. In embodiments, the cancer is melanoma. In embodiments, the cancer is bladder cancer.

In embodiments, the disease is an autoimmune disorder. In embodiments, the autoimmune disorder is rheumatoid arthritis. In embodiments, the autoimmune disorder is inflammatory bowel disease. In embodiments, the autoimmune disorder is diabetes.

In embodiments, the disease is caused by a bacterial infection. In embodiments, the bacterial infection is infection by Yersinia enterocolitica. In embodiments, the disease is caused by a viral infection. In embodiments, the disease is hepatitis.

In an aspect is provided a method of treating or preventing inflammation in a subject in need thereof, the method including administering to the subject an effective amount of a HVEM protein or fragment provided herein including embodiments thereof, a nucleic acid provided herein including embodiments thereof, a CAR provided herein including embodiments thereof, a nucleic acid provided herein including embodiments thereof, or a T-cell provided herein including embodiments thereof. In embodiments, the method includes administering to the subject an effective amount of a HVEM protein or fragment thereof provided herein including embodiments thereof. In embodiments, the method includes administering to the subject an effective amount of a nucleic acid provided herein including embodiments thereof. In embodiments, the method includes administering to the subject an effective amount of a CAR provided herein including embodiments thereof. In embodiments, the method includes administering to the subject an effective amount of a T-cell provided herein including embodiments thereof.

In embodiments, the inflammation is acute inflammation caused by a viral infection. In embodiments, the inflammation is acute inflammation caused by a bacterial infection. In embodiments, the bacterial infection is infection by Yersinia enterocolitica.

In embodiments, the inflammation is caused by asthma, rheumatoid arthritis, hepatitis, inflammatory bowel disease, transplant rejection, diabetes, obesity, or autoimmune disease.

### Embodiments

Embodiment 1. A herpes virus entry mediator (HVEM) protein or fragment thereof, comprising at least one of the following substitutions: H86D, L90A, L90D, L90R, A76D, T82D, Y83A, T84D, T84F, N88A, N88D, G89D, L94A, L94D at a position corresponding to the amino acid sequence of SEQ ID NO: 11; or H86D, L90A, L90D, L90R, E76D, T82D, Y83A, 184D, 184F, N88A, N88D, G89D, L94A, or L94D at a position corresponding the amino acid sequence of SEQ ID NO: 14.

Embodiment 2. The HVEM protein or fragment thereof of embodiment 1, comprising a H86A and L90A substitution, a H86D and L90A substitution, a H86D and L90D substitution, a H86D and L90R substitution, or a H86R and L90A substitution at positions corresponding to the amino acid sequence of SEQ ID NO: 11.

Embodiment 3. The HVEM protein or fragment thereof of embodiment 1, comprising a H86D and a L90A substitution, a H86D and a L90D substitution, or a H86D and a L90R substitution at a position corresponding the amino acid sequence of SEQ ID NO: 14.

Embodiment 4. The HVEM protein or fragment thereof of embodiment 1, comprising the amino acid sequence of SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, or SEQ ID NO: 54.

Embodiment 5. The HVEM protein or fragment thereof of embodiment 1 or 2, comprising the amino acid sequence of SEQ ID NO: 43, SEQ ID NO: 44, SEQ ID NO: 45, SEQ ID NO: 46, or SEQ ID NO: 47.

Embodiment 6. The HVEM protein or fragment thereof of embodiment 1, comprising an A76D, a H86D and a L90A substitution at positions corresponding to the amino acid sequence of SEQ ID NO: 11; or a A76D, a H86D and a L90D substitution at positions corresponding to the amino acid sequence of SEQ ID NO: 11.

Embodiment 7. The HVEM protein or fragment thereof of embodiment 6, comprising the amino acid sequence of SEQ ID NO: 48 or SEQ ID NO: 49.

Embodiment 8. The herpes virus entry mediator (HVEM) protein or fragment thereof of any one of embodiments 1-7, further comprising a delivery vehicle.

Embodiment 9. A nucleic acid encoding the HVEM protein or fragment thereof of any one of embodiments 1-7.

Embodiment 10. The nucleic acid encoding the HVEM protein or fragment thereof of embodiment 9, further comprising an expression vector.

Embodiment 11. A Chimeric Antigen Receptor (CAR) comprising: a) an ectodomain of the HVEM protein or fragment thereof of any one of embodiments 1-7, and b) a transmembrane domain.

Embodiment 12. The CAR of embodiment 11, further comprising an intracellular T-cell signaling domain.

Embodiment 13. The CAR of embodiment 12, wherein the intracellular T-cell signaling domain is a CD3 (intracellular T-cell signaling domain.

Embodiment 14. The CAR of any one of embodiments 11-13, further comprising an intracellular co-stimulatory T-cell signaling domain.

Embodiment 15. The CAR of embodiment 14, wherein said intracellular co-stimulatory signaling domain is a CD28 intracellular co-stimulatory signaling domain, a 4-1BB intracellular co-stimulatory signaling domain, a ICOS intracellular co-stimulatory signaling domain, or an OX-40 intracellular co-stimulatory signaling domain.

Embodiment 16. A nucleic acid encoding a CAR of any of embodiments 11-15.

Embodiment 17. A T-cell comprising the CAR of any of embodiments 11-15.

Embodiment 18. A method of treating or preventing an autoimmune disorder in a subject in need thereof, said method comprising administering to said subject an effective amount of the HVEM protein or fragment thereof of any one of embodiments 1-8, the nucleic acid encoding the HVEM protein or fragment thereof of embodiment 9 or 10, the CAR of any one of embodiments 11-15, the nucleic acid encoding the CAR of embodiment 16, or the T-cell of embodiment 17.

Embodiment 19. A method of treating or preventing inflammation in a subject in need thereof, said method comprising administering to said subject an effective amount of the HVEM protein or fragment thereof of any one of embodiments 1-8, the nucleic acid encoding the HVEM protein or fragment thereof of embodiment 9 or 10, the CAR of any one of embodiments 11-15, the nucleic acid encoding the CAR of embodiment 16, or the T-cell of embodiment 17.

Embodiment 20. The method of embodiment 19, wherein said inflammation is acute inflammation caused by a viral infection.

Embodiment 21. A herpes virus entry mediator (HVEM) protein or fragment thereof, comprising at least one of the following substitutions: G72A, G72D, G72R, T73A, T73D, or V74D at a position corresponding to the amino acid sequence of SEQ ID NO: 11; or G72D, T73A, or V74E at a position corresponding the amino acid sequence of SEQ ID NO: 14.

Embodiment 22. The HVEM protein or fragment thereof of embodiment 21, comprising a G72A and a V74D substitution, a G72D and a V74A substitution, a G72D and a V74D substitution, a G72D and a V74R substitution, or a G72R and a V74A substitution at positions corresponding to the amino acid sequence of SEQ ID NO: 11.

Embodiment 23. The HVEM protein or fragment thereof of embodiment 21, comprising a G72D and a V74E substitution at positions corresponding to the amino acid sequence of SEQ ID NO: 14.

Embodiment 24. The HVEM protein or fragment thereof of embodiment 21, comprising the amino acid sequence of SEQ ID NO: 18, SEQ ID NO: 19, SEQ ID NO: 20, SEQ ID NO: 21, SEQ ID NO: 22, SEQ ID NO: 23, SEQ ID NO: 38, SEQ ID NO: 39, SEQ ID NO: 40, SEQ ID NO: 41, SEQ ID NO: 42, SEQ ID NO: 51, SEQ ID NO: 52, or SEQ ID NO: 53.

Embodiment 25. The HVEM protein or fragment thereof of embodiment 21 or 22, comprising the amino acid sequence of SEQ ID NO: 38, SEQ ID NO: 39, SEQ ID NO: 40, SEQ ID NO: 41, or SEQ ID NO: 42.

Embodiment 26. The HVEM protein or fragment thereof of any one of embodiments 21-25, further comprising a delivery vehicle.

Embodiment 27. The HVEM protein or fragment thereof of embodiment 26, wherein said delivery vehicle is an Fc domain, a nanoparticle, or a liposome.

Embodiment 28. A nucleic acid encoding the HVEM protein or fragment thereof of any one of embodiments 21-25.

Embodiment 29. The nucleic acid encoding the HVEM protein or fragment thereof of embodiment 28, further comprising an expression vector.

Embodiment 30. A Chimeric Antigen Receptor (CAR) comprising: a) an ectodomain of the HVEM protein or fragment thereof of any one of embodiments 21-25, and b) a transmembrane domain

Embodiment 31. The CAR of embodiment 30, further comprising an intracellular T-cell signaling domain.

Embodiment 32. The CAR of embodiment 31, wherein the intracellular T-cell signaling domain is a CD3 (intracellular T-cell signaling domain.

Embodiment 33. The CAR of any one of embodiments 30-32, further comprising an intracellular co-stimulatory T-cell signaling domain.

Embodiment 34. The CAR of embodiment 33, wherein said intracellular co-stimulatory signaling domain is a CD28 intracellular co-stimulatory signaling domain, a 4-1BB intracellular co-stimulatory signaling domain, a ICOS intracellular co-stimulatory signaling domain, or an OX-40 intracellular co-stimulatory signaling domain.

Embodiment 35. A nucleic acid encoding the CAR of any of embodiments 30-34.

Embodiment 36. A T-cell comprising a CAR of any of embodiments 30-34.

Embodiment 37. A method of treating or preventing a disease in a subject in need thereof, said method comprising administering to said subject an effective amount of the HVEM protein or fragment thereof of any one of embodiments 21-27, the nucleic acid encoding the HVEM protein or fragment thereof of embodiment 28 or 29, the CAR of any of embodiments 30-34, the nucleic acid encoding the CAR of embodiment 35, or the T-cell of embodiment 36.

Embodiment 38. The method of embodiment 37, wherein said disease is cancer.

Embodiment 39. The method of embodiment 37, wherein said disease is an autoimmune disorder.

### Examples

### Example 1: Introduction to Exemplary Studies

### Abstract

HVEM is a TNF (tumor necrosis factor) receptor contributing to a broad range of immune functions involving diverse cell types. It interacts with a TNF ligand, LIGHT, and immunoglobulin (Ig) superfamily members BTLA and CD160. Assessing the functional impact of HVEM binding to specific ligands in different settings has been complicated by the multiple interactions of HVEM and HVEM binding partners. To dissect the molecular basis for multiple functions, the inventors determined crystal structures that reveal the distinct HVEM surfaces that engage LIGHT or BTLA/CD160, including the human HVEM:LIGHT:CD160 ternary complex, with HVEM interacting simultaneously with both binding partners. Based on these structures, the inventors generated mouse HVEM mutants that selectively recognized either the TNF or Ig ligands in vitro. Knock-in mice expressing these muteins maintain expression of all the proteins in the HVEM network, yet they demonstrate selective functions for LIGHT in the clearance of bacteria in the intestine and for the Ig ligands in the amelioration of liver inflammation.

### INTRODUCTION

Members of the tumor necrosis factor receptor super family (TNFRSF) regulate diverse processes, but in several cases understanding these processes is hampered by the ability of receptors and ligands to bind to multiple partners. One prominent example is provided by the herpes virus entry mediator (HVEM), or TNFRSF14, initially identified as important for entry of herpes simplex virus (HSV) through recognition of HSV glycoprotein D (gD). Subsequently, a tumor necrosis factor super family (TNFSF) ligand for the herpes virus entry mediator (HVEM) was characterized, known as LIGHT (homologous to lymphotoxin, exhibits inducible expression and competes with HSV glycoprotein D for binding to herpesvirus entry mediator, a receptor expressed on T lymphocytes) or TNFSF14 (12, 13). Engagement of HVEM by LIGHT was implicated in multiple responses. For example, in T lymphocytes, it stimulates proliferation, a cytokine production, and the development of CD8 T cell memory (17, 12, 13 and 38). LIGHT also engaged HVEM to stimulate the cytokine production by type 3 innate lymphoid cells (ILC3) (34) and in keratinocytes it bounds HVEM to stimulate periostin, contributing to atopic dermatitis (15).

LIGHT also binds to another TNFRSF member, a lymphotoxin-beta receptor (LTβR or TNFRSF3), which is expressed by stromal and myeloid lineages. This interaction regulates lymph node formation, dendritic cell migration (47), and IL-12 production by a dendritic cell (DC) (28). The LIGHT-LTβR interaction also has been reported to induce apoptosis of cancer cells (46), is important for macrophage activity in wound healing (31) and influences lipid metabolism by regulating hepatic lipase expression in hepatocytes (4, 21). Furthermore, LIGHT participates in additional processes in which a specific receptor had not been implicated, including the resolution of inflammation in an experimental autoimmune encephalomyelitis (23), the induction of adipocyte differentiation (40), and the induction of osteoclastogenic signals (2, 13).

HVEM also binds with immunoglobulin superfamily (IgSF) molecules B and T lymphocyte attenuator (BTLA or CD272) and CD160. HVEM engages in bidirectional signaling, serving not only as a receptor, but potentially as a ligand for IgSF receptor signaling (37). HVEM: BTLA engagement delivers an overall inhibitory immune response (27), while the interaction between HVEM and CD160 on T cells might either attenuate the activities of specific subsets of CD4 T lymphocytes or enhance the activity of CD8 T cells (3;39). Notably, engagement of CD160 by HVEM also controls the cytokine production by natural killer (NK) cells and is important for mucosal immunity (36, 42, 44). Furthermore, HVEM was reported to interact with a synaptic adhesion-like molecule 5 (SALM5), mainly expressed in brain, to confer immune-privilege in the central nervous system (48). These different interactions are summarized in FIG. 7. CD160 also binds to some major histocompatibility complex (MHC) class I molecules (Le Bouteiller et al., 2002, 22), further expanding the complexity of this protein-protein interaction network.

The promiscuous interactions of HVEM pose challenges for characterizing the mechanistic contributions of the HVEM-associated pathways in different immune responses and diseases. Conditional knockouts might isolate effects in particular cell types, but elimination of expression of one protein, for example LIGHT, not only abolishes the LIGHT-HVEM binding, but also eliminates the LIGHT-LTβR binding and may also indirectly affect HVEM interactions with its IgSF ligands by altering the availability of HVEM (37). This complexity may make it difficult to reach definitive conclusions about the relevant binding partners responsible for a phenotype and may account for circumstances in which the phenotypes in whole body receptor and corresponding ligand knockouts did not agree (11).

Herein, in order to better understand this receptor-ligand network, the inventors set out to test mutants of HVEM with selective ligand binding. Based on multiple crystal structures, including the human HVEM:LIGHT:CD160 ternary complex, extensive epitope mapping and engineering of selective mHVEM mutants were performed. HVEM muteins were expressed in mice to show definitively that selective HVEM-ligand interactions were important in resistance to mucosal bacterial infection and in prevention of liver inflammation in a context where all members of the protein network were present and only selective interactions were disrupted.

### Example 2: Results

### Human HVEM:LIGHT Complex Exists as a 3:3 Assembly

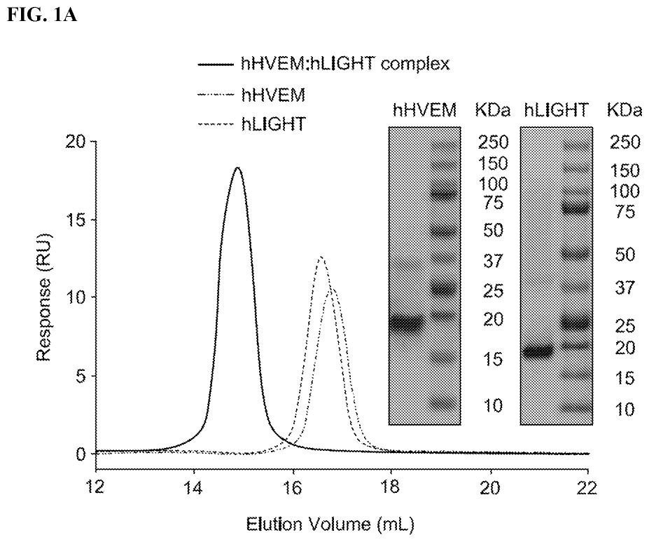

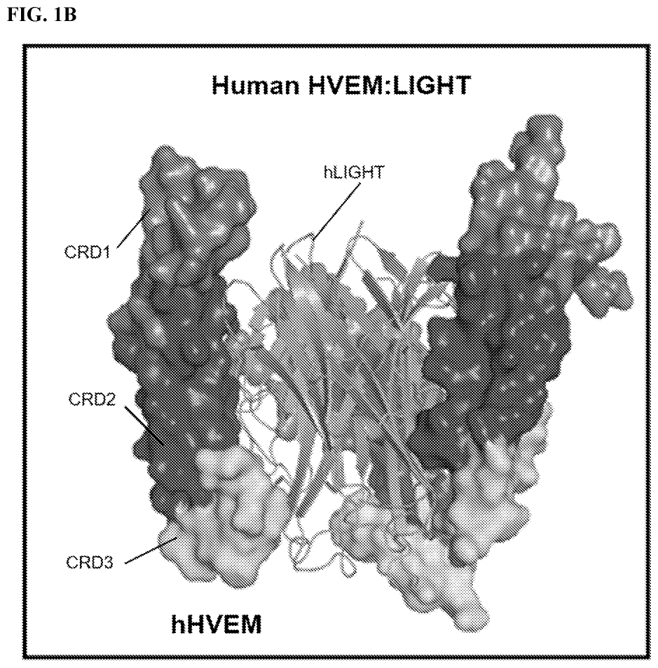

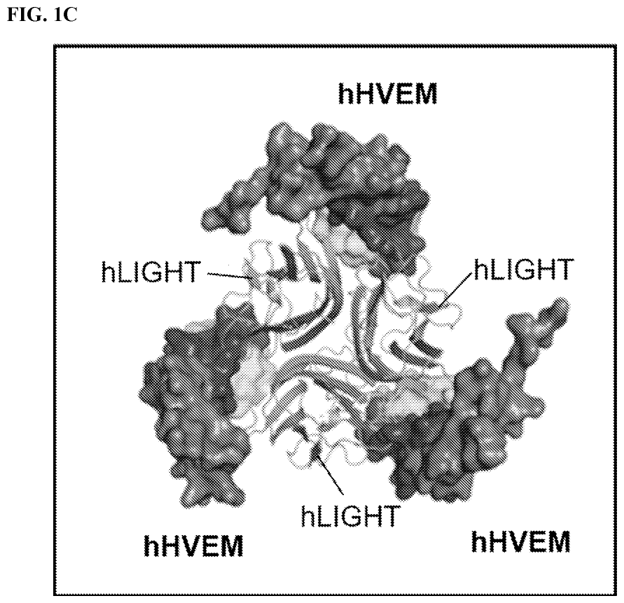

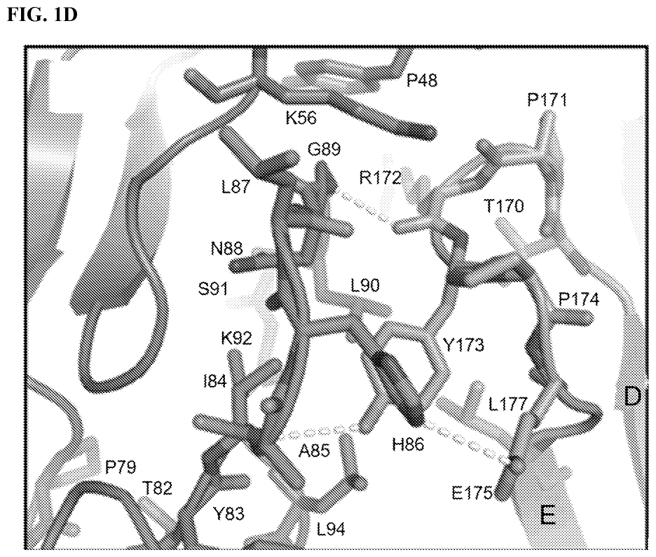

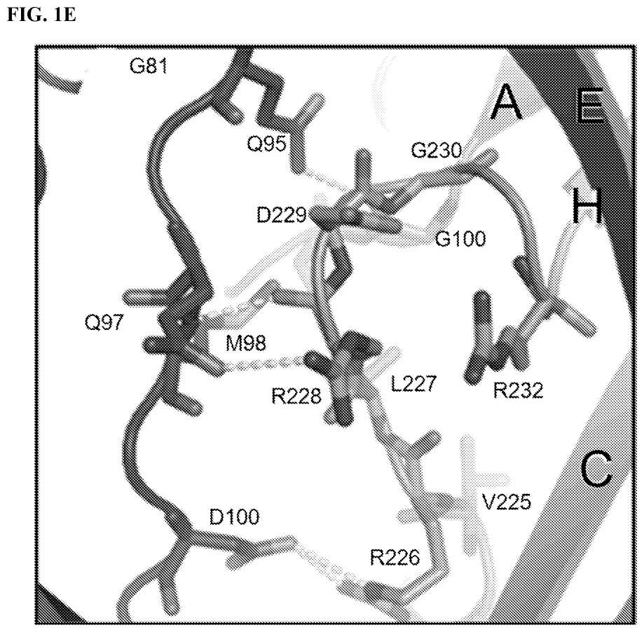

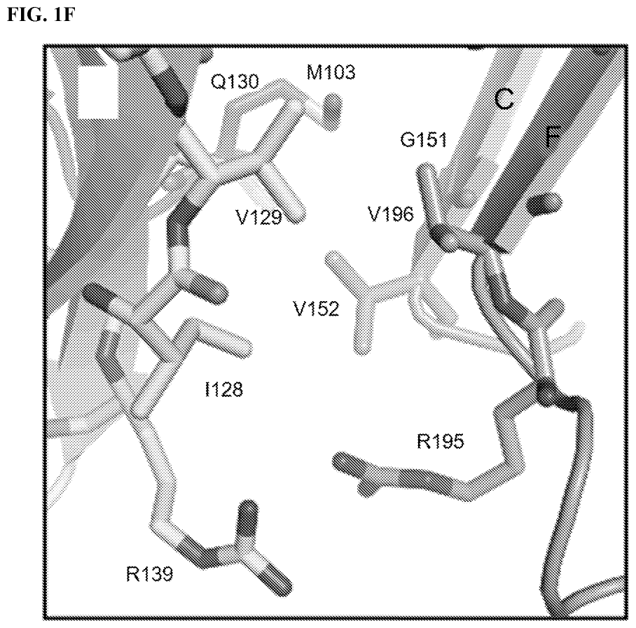

The extracellular domains of human LIGHT (denoted as hLIGHT; ˜18 KDa for the monomer and ˜54 KDa for the homotrimer) and human HVEM (denoted as hHVEM; ˜15 KDa) were purified to homogeneous, monodisperse species as indicated by an analytical size exclusion chromatography (SEC) (FIG. 1A). Mixing equal molar equivalents of hLIGHT and hHVEM monomers, resulted in a single species with an apparent molecular weight of ˜100 KDa, consistent with the formation of a 3:3 stoichiometric hHVEM:hLIGHT assembly in solution (FIGS. 1A-1C).

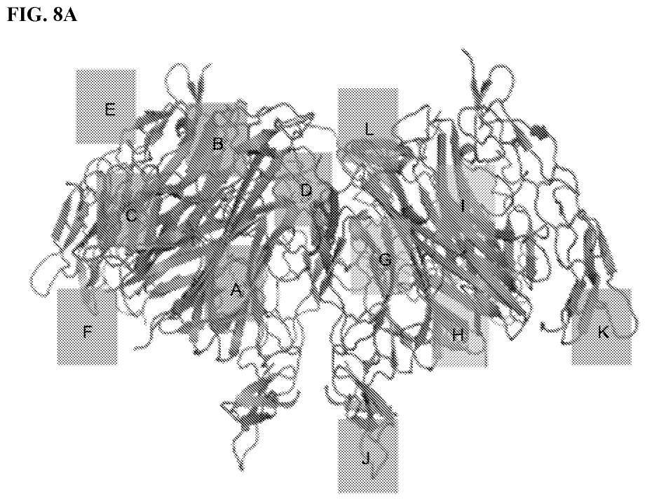

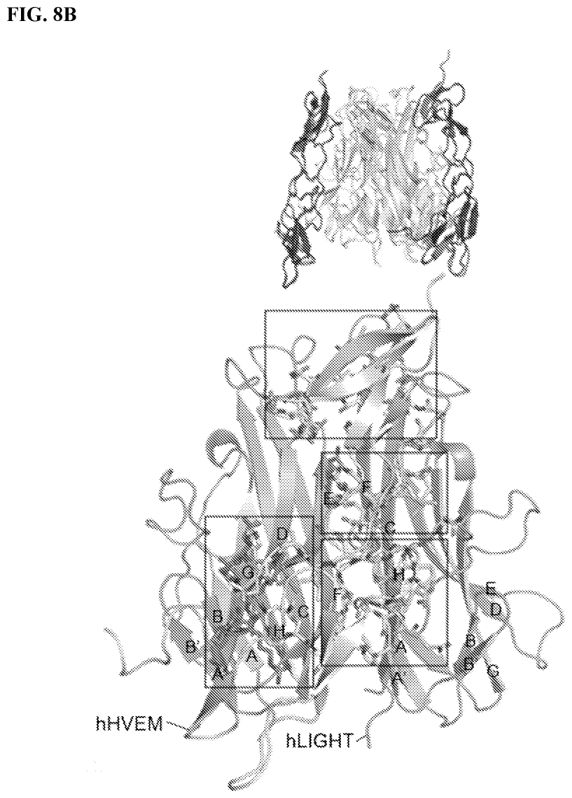

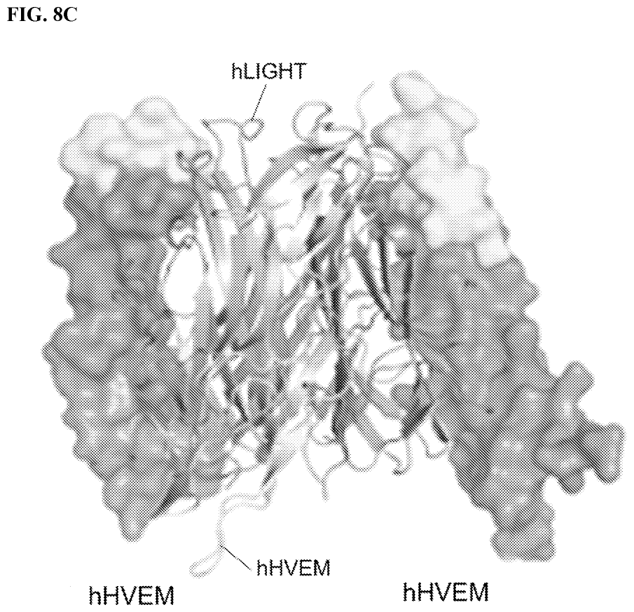

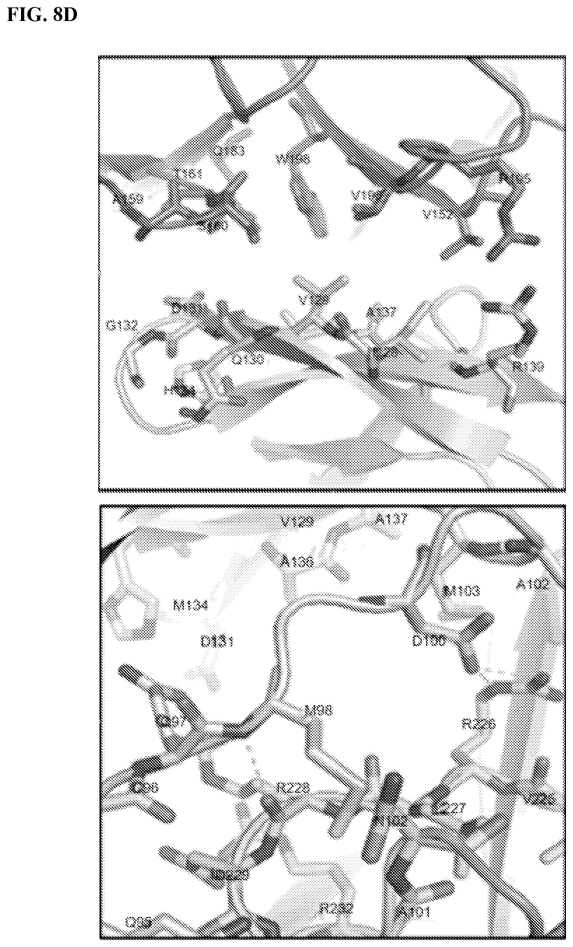

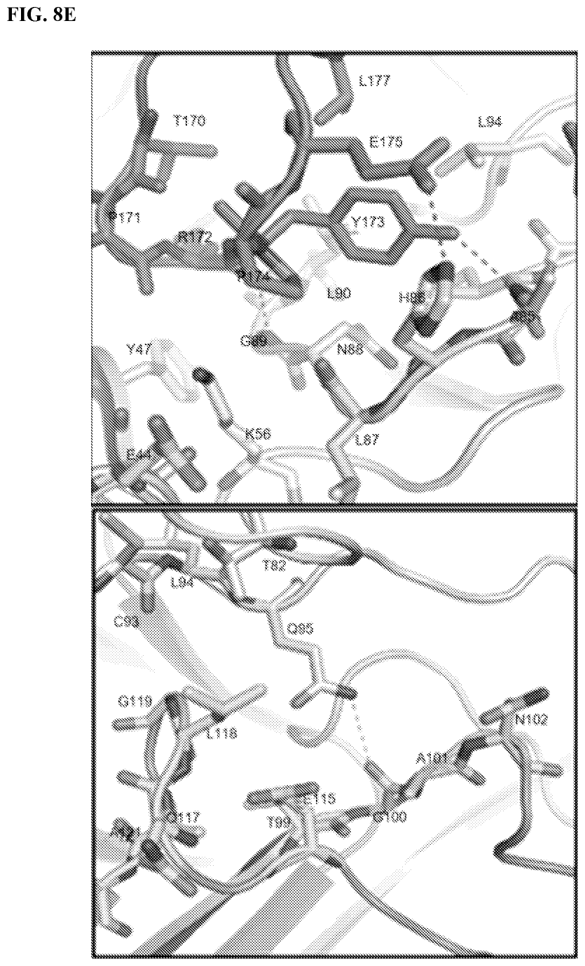

The crystal structure of the hHVEM:hLIGHT complex was determined to the resolution of 2.30 Å by molecular replacement using Protein Data Bank (PDB) entries 4KG8 (hLIGHT) and 4FHQ (hHVEM) as starting search models (Table 3). The asymmetric unit of the hHVEM:hLIGHT crystals contains six independent chains of hLIGHT and six independent chains of hHVEM, which formed two classical 3:3 TNF:TNFR hexameric assemblies with three-fold symmetry (FIGS. 8A-8C); a single 3:3 TNF:TNFR hexameric assembly was consistent with SEC analysis. The hHVEM ectodomain was composed of four cysteine rich domains (CRDs), while hLIGHT formed a compact homotrimeric structure. In the hexameric assembly, CRD1, CRD2 and CRD3 of hHVEM engaged hLIGHT via surfaces contributed by two adjacent hLIGHT protomers (FIG. 1B and FIG. 8C). The two independent hHVEM:hLIGHT hexameric complexes exhibited similar overall structures with a RMSD of 1.8 Å for 742 aligned C atoms. The regions with the greatest structural divergence resided in the N- and C-termini of the proteins, which did not directly contribute to the binding interface. The hHVEM:hLIGHT recognition interfaces were highly similar within and between the two complexes (FIG. 8B), and the following discussion was based on the hLIGHT G and H chains, and hHVEM J chain (FIG. 8A).

TABLE 3
Data Collection and Refinement Statistics

| | | |
|---|---|---|
| hHVEM:hLIGHT | hHVEM:hLIGHT:hCD160 | mHVEM |

Data Collection

| Wavelength used | 1.075 | | 0.97931 | | 0.97931 | |
|---|---|---|---|---|---|---|
| (Å) | | | | | | |
| Resolution range | 2.30-50.00 | | 3.50-50.00 | | 2.10-50.00 | |
| (Å) | (2.30-2.34) | | (3.50-3.83) | | (2.10-2.14) | |
| Space group | P2₁2₁2₁ | | I23 | | P4₁2₁2 | |
| Unit cell (Å) | a = 111.7, | | a = b = c = 214.7 | | a = b = 64.7, | |
| | b = 113.6, | | | | c = 69.0 | |
| | c = 163.3 | | | | | |
| Unique | 92792 | | 20868 | | 8989 | |
| reflections (N) | | | | | | |
| Redundancy | 10.8 | (10.7) | 20.7 | (17.9) | 13.5 | (9.9) |
| Completeness | 99.9 | (99.7) | 99.9 | (100) | 99.5 | (99.1) |
| I/sigma | 22.7 | (3.1) | 16.1 | (2.2) | 17.1 | (2.2) |
| R_{merge} | 0.125 | (0.936) | 0.191 | (1.674) | 0.135 | (0.938) |

| | | | | | |
|---|---|---|---|---|---|
| CC_{1/2} | N/A | 0.999 | (0.676) | 0.999 | (0.943) |

Refinement

| Resolution range | 2.30-48.92 | | 3.50-19.93 | | 2.10-20.00 | |
|---|---|---|---|---|---|---|
| (Å) | (2.30-2.36) | | (3.50-3.59) | | (2.10-2.16) | |
| R_{work} | 0.188 | (0.245) | 0.257 | (0.370) | 0.212 | (0.355) |
| R_{free} | 0.231 | (0.270) | 0.285 | (0.293) | 0.257 | (0.328) |

| | | | |
|---|---|---|---|
| Average B factor | 38.4 | 139.9 | 55.5 |
| (Å²) | | | |
| Rms bond (Å) | 0.021 | 0.005 | 0.018 |
| Rms angles (°) | 2.081 | 1.290 | 1.928 |
| PDB code | 4RSU | 7MSG | 7MSJ |

R_{merge} = ΣₕₖₗΣᵢ|Iᵢ(hkl) − <I(hkl)>|/ΣₕₖₗΣᵢIᵢ(hkl).
R_{work} = Σ|F_{c} − Fₒ|/ΣFₒ.
Parentheses indicate statistics for the highest resolution bin.

### The Binding Interface Between Human HVEM and LIGHT

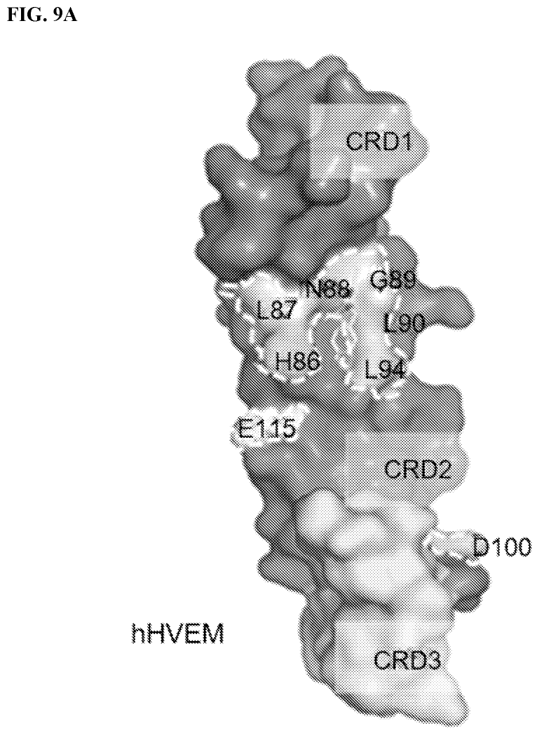

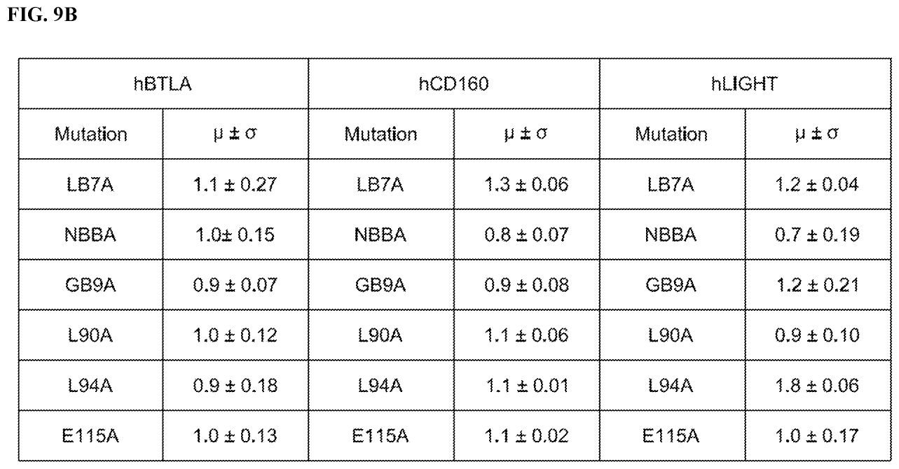

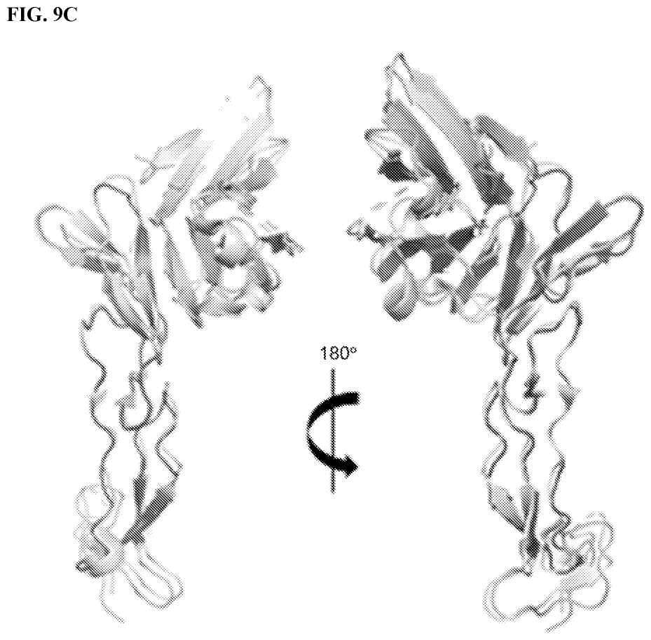

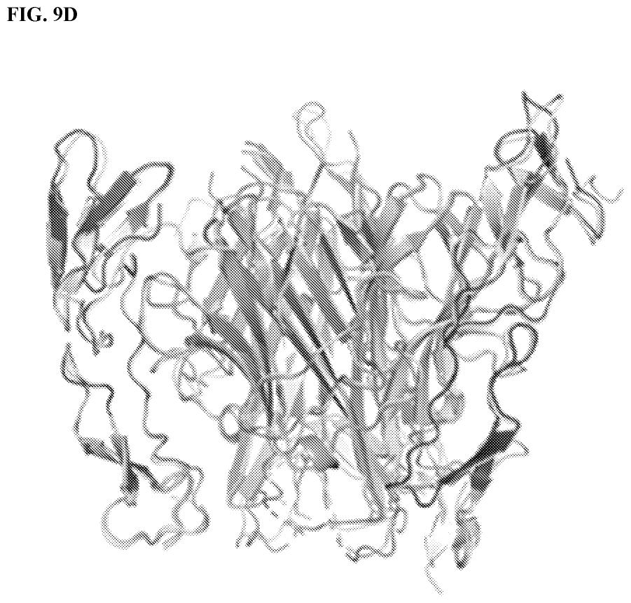

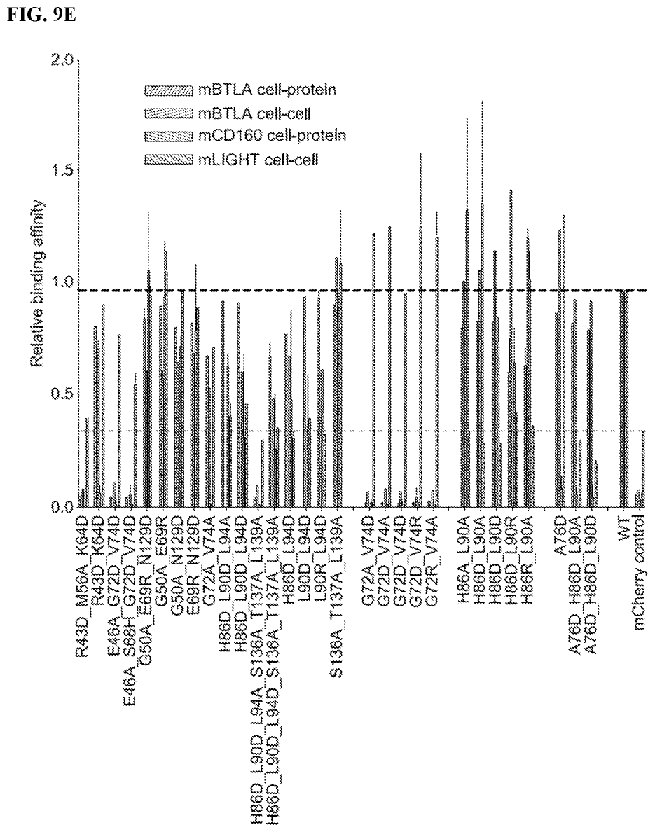

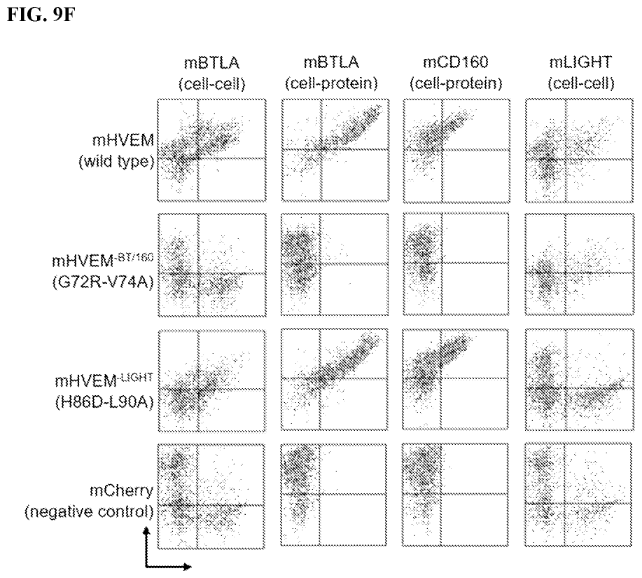

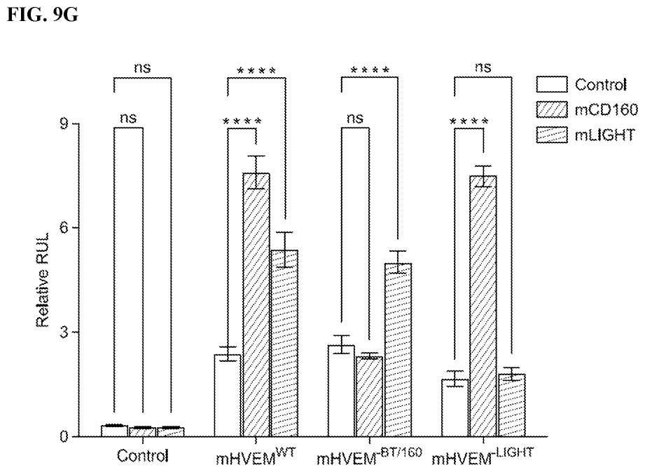

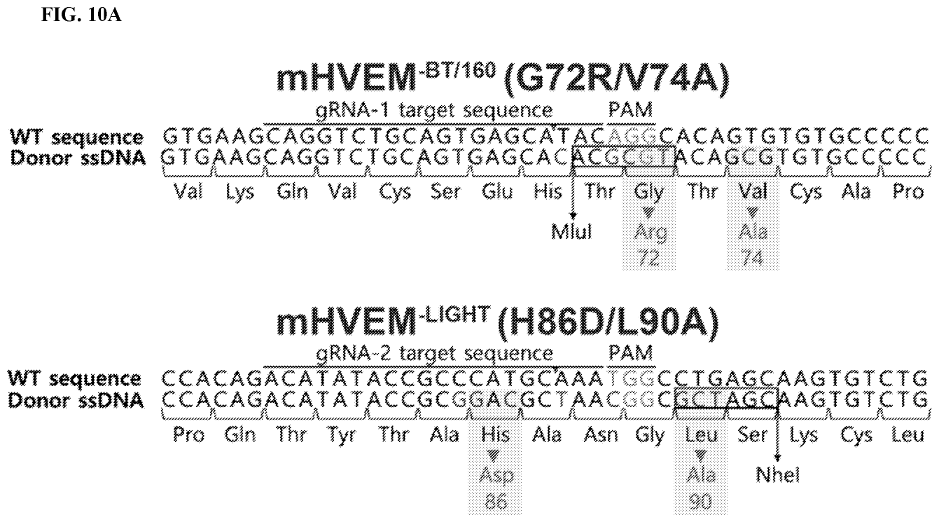

The structure of the hHVEM:hLIGHT complex showed that HVEM CRDI and CRD2 domains interacted with the DE, AA′ and GH loops of LIGHT, while HVEM CRD3 interacted with LIGHT CD and EF loops (FIGS. 1D-1F and FIGS. 8C-8D). The interaction between the hHVEM CRD2 and the hLIGHT DE loop appeared to be important for human HVEM:LIGHT recognition, as it contributed multiple potential polar contacts. The main chain amide group of hHVEM A85 (position numbered with initiation codon=1) formed a hydrogen bond with the side chain hydroxyl group of hLIGHT Y173 (FIG. 1D and FIG. 8D), consistent with the behavior of the Y173F mutation in hLIGHT, which significantly diminished the binding of hLIGHT with hHVEM (32). Human HVEM N88 did not directly contact hLIGHT Y173, but was relatively close, and the hHVEM N88A mutation attenuated binding to hLIGHT (FIGS. 9A-9D). Notably, the residues analogous to LIGHT Y173 in FasL, TL1A, TRAIL, TNFα and LTα were conserved and these tyrosines were also important for DE loop mediated receptor binding, whereas the homologous residues in RANKL, OX40L, CD40L and 4-1BBL were not tyrosines and were not critical for receptor binding, indicating diverse mechanisms of binding among different TNF ligands and receptors. The hHVEM G89 main chain amide group formed a hydrogen bond with the main chain oxygen of hLIGHT R172 (FIG. 1D and FIG. 8D). HVEM H86 side chain imidazole functionality made a polar contact with the side chain carboxyl group of hLIGHT E175 (FIG. 1D and FIG. 8D). It was previously reported that the hHVEM H86I mutation dramatically reduced binding to hLIGHT (35).

Human HVEM CRD2 formed four additional polar contacts with GH loop of hLIGHT (FIG. 1E and FIG. 8D). The hHVEM Q97 side chain oxygen formed a polar contact with hLIGHT R228 side chain. Human HVEM M98 backbone amide group contacted the backbone oxygen of hLIGHT R228 and the side chain carboxyl group of hHVEM D100 formed two polar contacts with the side-chain guanidinium group of hLIGHT R226 (FIG. 1E and FIG. 8D). The hHVEM D100R mutation resulted in undetectable binding with hLIGHT (35). The AA′ loop from the lower region of CRD2 contributes only a single polar contact, formed by the main chain oxygen from G100 of hLIGHT and the side chain amide group of hHVEM Q95 (FIG. 1E and FIG. 8D).

hHVEM CRD3 residues, including 1128-G132, H134 and A136-R139 participated in interactions with G151-V152 and A159-T161 from the CD loop, as well as residues Q183, R195-V196 and W198 from the EF loop of hLIGHT (FIGS. 8C-8D). Examination of the structure in this region revealed no polar contacts between hHVEM and hLIGHT. A modest hydrophobic interface was formed by the packing of the side chains of hHVEM residues 1128 and V129 against the side chains of hLIGHT V152 and V196 (FIG. 1F and FIG. 8D).

### Structure of the Human HVEM:LIGHT:CD160 Ternary Complex

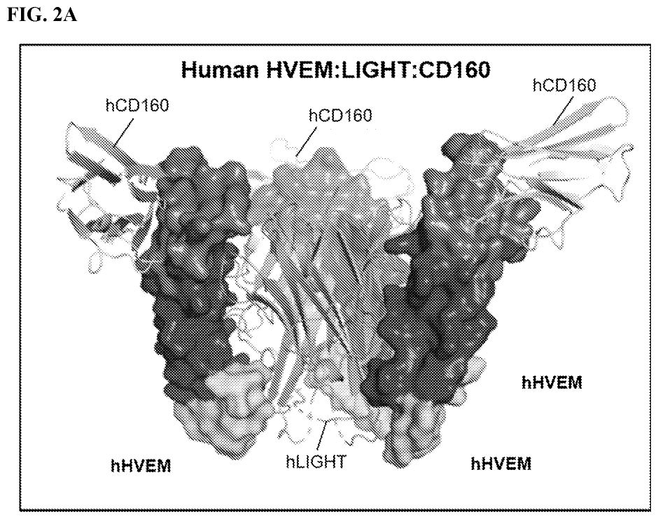

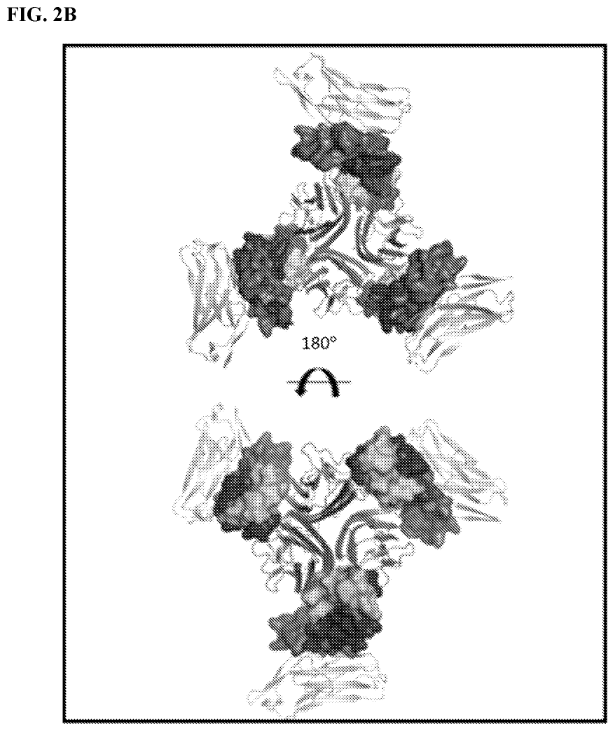

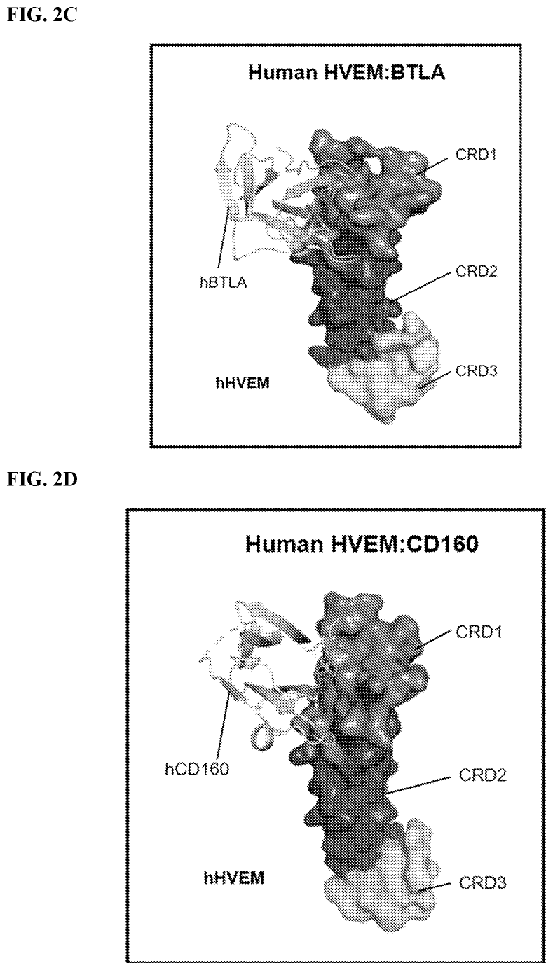

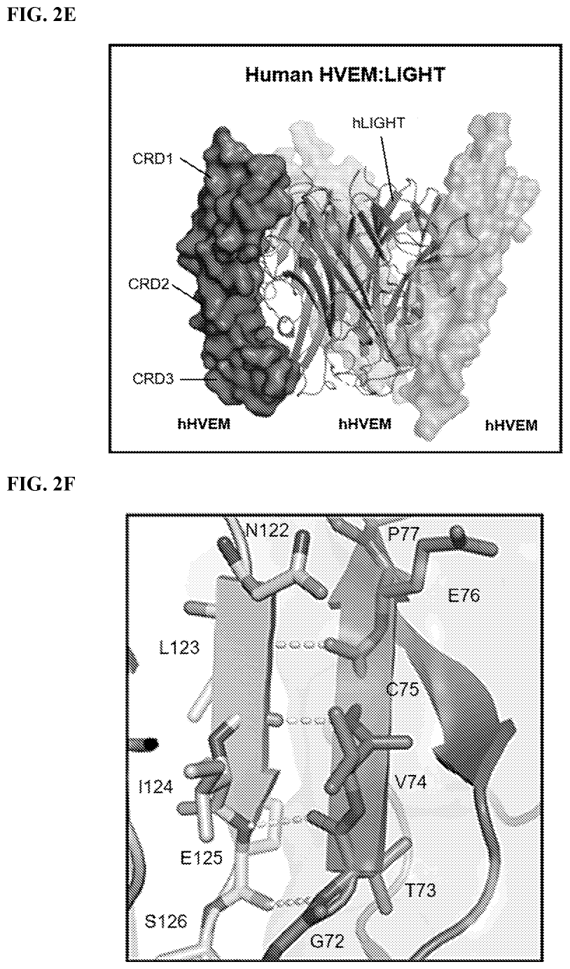

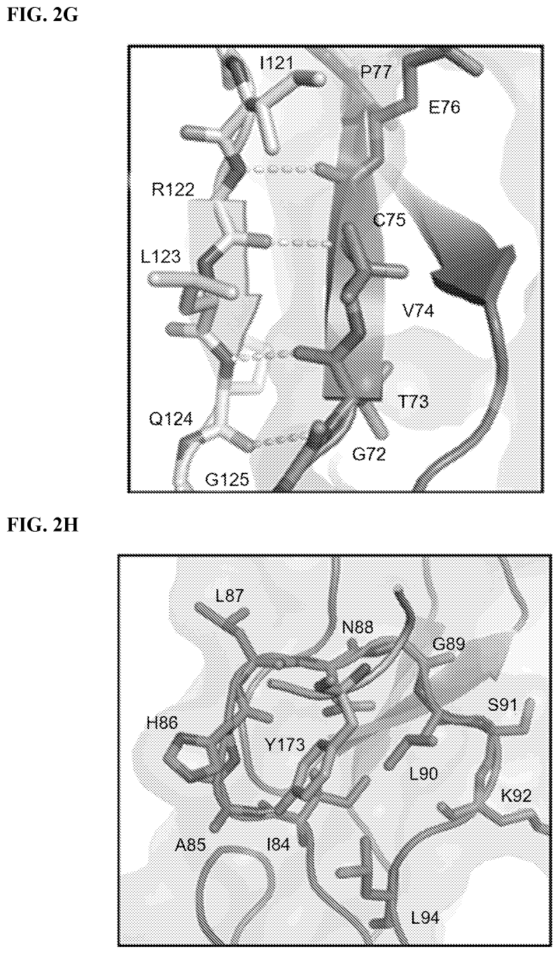

It was previously shown that LIGHT and the IgSF ligands did not compete for binding to HVEM (3, 19) suggesting the potential for forming a ternary complex. Therefore, a set out was made to solve the crystal structure of hHVEM:hLIGHT:hCD160 (human CD160 was denoted as hCD160) complex (PDB entry 7MSG). Accordingly, it was determined that the structure of this complex to 3.5 Å resolution by molecular replacement using CD160 (PDB entry 6NG9) and the hHVEM:hLIGHT complex described above (PDB entry 4RSU) as search models (FIGS. 2A-2B). The asymmetric unit contained three copies of each hHVEM, hLIGHT and hCD160, forming a ternary complex with 3:3:3 stoichiometry. Within the ternary assembly, hHVEM and hLIGHT exhibited the classical 3:3 TNF:TNFR assembly, with contacts that were very similar to the structure of the hHVEM:hLIGHT binary complex described above. The hHVEM:hLIGHT complex formed the core of the ternary complex with each hHVEM CRDI further binding a single molecules of hCD160 in a manner similar to that observed in the structure of the hHVEM:hCD160 binary complex (FIG. 2A and FIG. 2D and FIG. 9C). Notably, the structures of hHVEM:hLIGHT:hCD160 and hHVEM:hCD160 complexes relied on the use of a single chain hCD160-hHVEM fusion protein as the relatively weak interaction of hCD160-hHVEM (7.1±0.9 uM) did not support the stable complex formation in solution (19). The crystal structure of the hHVEM:hLIGHT:hCD160 complex provided direct evidence that hLIGHT and hCD160 may simultaneously engage hHVEM, resulting in a higher order assembly with the potential of coordinated signaling through both hHVEM and hCD160. Notably, the simultaneous interaction of hCD160 and hLIGHT with hHVEM altered the local organization of hCD160, as engagement of hHVEM with trimeric hLIGHT might enforce close proximity of up to three hCD160 molecules with distinct geometric organization, as compared to the engagement of hCD160 and hHVEM in the absence of hLIGHT.

Crystal structures and complementary mutagenesis studies of hHVEM:hCD160 and hHVEM:hBTLA (human BTLA is denoted as hBTLA) complexes demonstrated that both hCD160 and hBTLA mainly bind to CRDI on hHVEM (FIGS. 2C-2D) (7, 19). In contrast, the crystal structure of the hHVEM:hLIGHT complex shows hLIGHT bound to CRD2, CRD3 and a small part of CRDI on hHVEM (FIG. 2E). Crystal structures of hHVEM in complex with hBTLA and hCD160 highlighted an anti-parallel intermolecular β-strand interaction, in which the β-strand composed of residues G72-P77 from CRDI in hHVEM contacts the edge β-strands in hBTLA and hCD160 through canonical main-chain-to-main-chain β-sheet hydrogen bonds (FIGS. 2F-2G). This pattern of hCD160 interactions with hHVEM was conserved in the ternary hHVEM:hLIGHT:hCD160 complex. Mutations of residues within this intermolecular β-strand (G72-P77) in HVEM CRDI significantly altered the binding affinities (35), while hHVEM CRD2 mutations did not significantly alter the affinities to hCD160 and hBTLA. In contrast, HVEM CRD2 mutations, particularly the HVEM residues formed the concave cavity surrounding hLIGHT Y173, significantly affected hHVEM:hLIGHT binding (FIG. 2H). Because both hCD160 and hBTLA bind to similar epitopes on hHVEM CRD1 (7, 19), it is likely that hHVEM, hLIGHT and hBTLA were able to form a ternary complex similar to the trimolecular complex of hHVEM:hLIGHT:hCD160 that was determined.

### Structure Guided Mutagenesis of Mouse HVEM Mutants

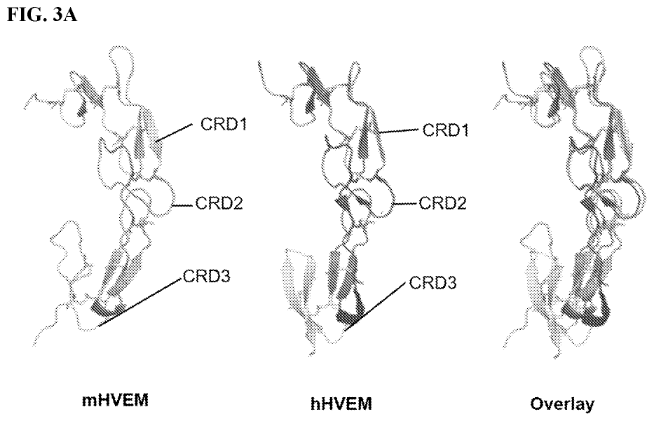

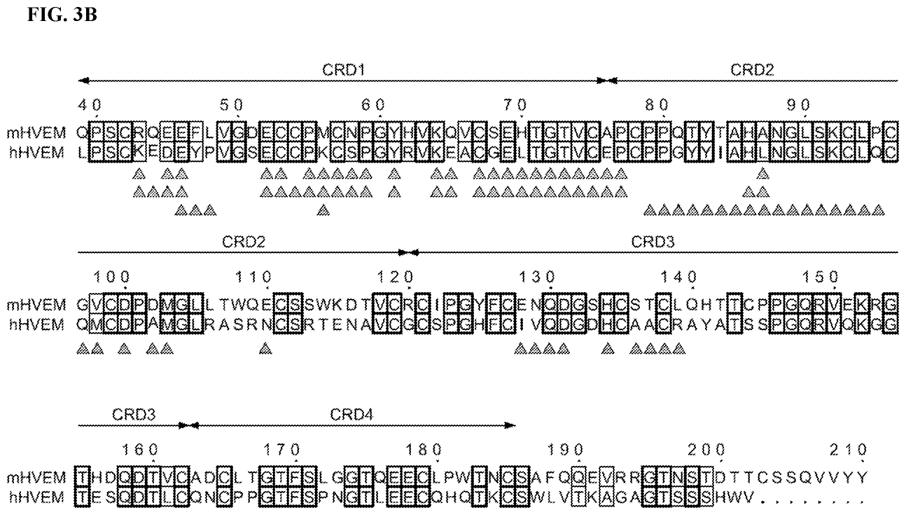

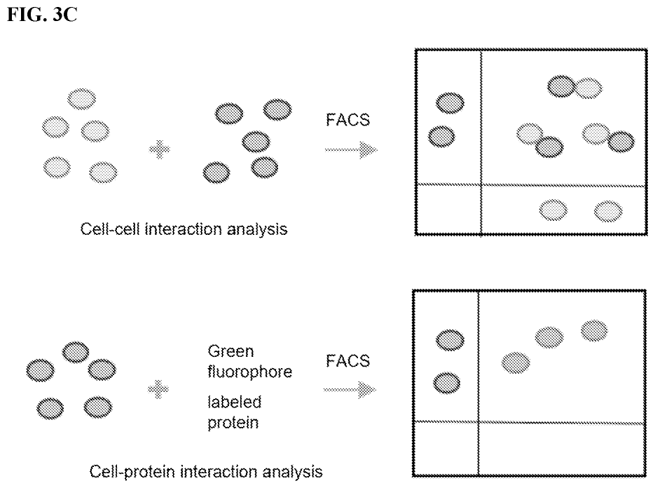

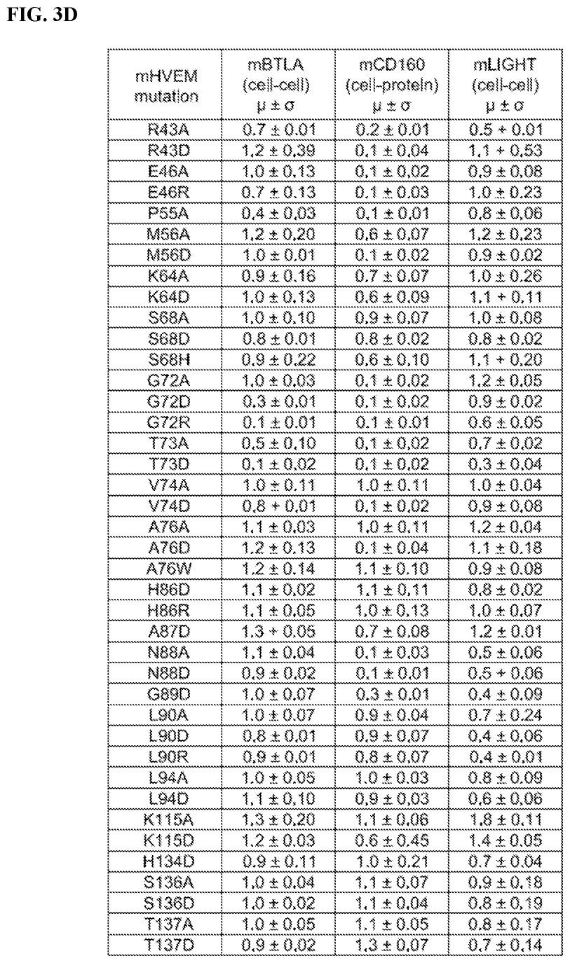

The mHVEM (mouse HVEM was denoted as mHVEM, PDB entry 7MSJ) structure was determined to 2.10 Å resolution by molecular replacement using the human HVEM (PDB entry 4FHQ) as the search model. The mouse and human HVEM structures are similar with RMSD of 2.7 Å for 97 aligned C_{□} atoms, with the biggest differences in CRD3 (FIGS. 3A-3B). Based on structural and sequence alignments between hHVEM and mHVEM, the solvent accessible mHVEM residues close to the putative binding interfaces were mutated to dissect the interaction network and enable in vivo HVEM functional studies.

The relative binding affinities of mHVEM mutants with mBTLA and mLIGHT (mouse BTLA and LIGHT were denoted as mBTLA and mLIGHT, respectively) were evaluated by a cell-cell interaction assay (FIG. 3C). The relative binding affinities of mHVEM mutants for mCD160 (mouse CD160 was denoted as mCD160) binding were screened using a cell-soluble protein assay because of low surface expression of the CD160 protein. A total of 52 mHVEM surface residues within or close to the likely ligand binding interfaces were individually mutated to different amino acids to probe the effect on ligand binding and to identify variants with selective ligand recognition (FIG. 3D). For example, alteration of mHVEM G72 or V74 to aspartic acid attenuated binding to both mBTLA and mCD160, but not binding to mLIGHT; the mHVEM R43D, M56D or A76D mutations decreased binding to mCD 160 but not mBTLA and mLIGHT; the mHVEM H86D, L90A, L94A and L94D mutations compromised the interaction with mLIGHT but not to mBTLA or mCD160 (FIG. 3D).

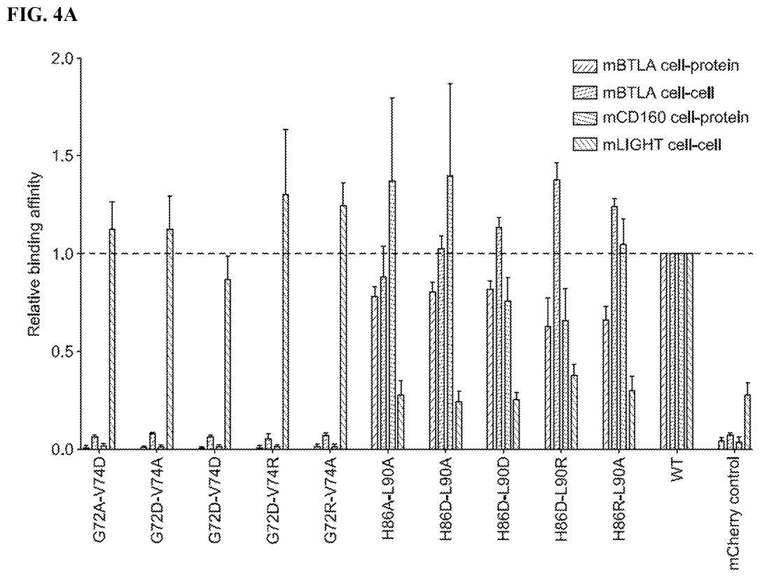

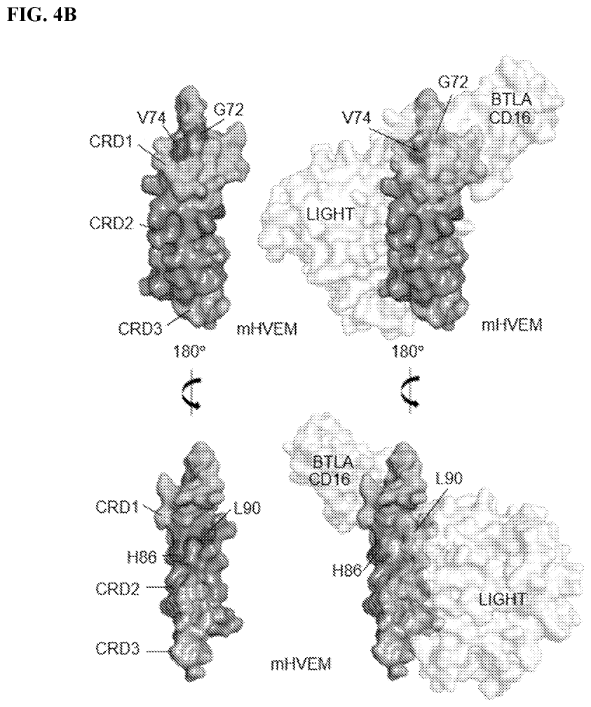

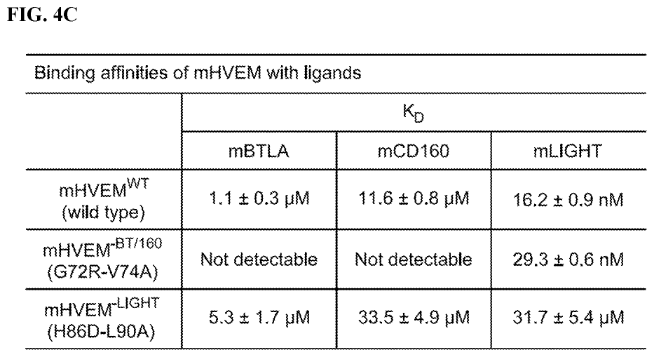

To further modulate the selectivity toward mLIGHT or mBTLA/mCD160, mHVEM mutations with similar binding properties were combined (FIG. 4A and FIG. 9E). For example, the combination of the G72 and V74 mutations completely eliminated binding to both mBTLA and mCD160, but did not appreciably impact mLIGHT binding in the flow cytometry based binding assays. Various pairwise combinations of mutations of H86, L90 and L94 eliminated mLIGHT binding, but did not substantially impact binding to mBTLA or mCD160 (FIG. 4A and FIGS. 9E-9G). Thus, these compound mutations resulted in several additional mHVEM variants with considerable binding selectivity. Although triple mutation of H86, L90 and L94 removed mLIGHT binding, it also dramatically reduced binding to mBTLA and mCD160 (FIG. 9E). Not surprisingly, other combinations of mutations also reduced the binding to all ligands, such as the mHVEM R43D-M56A-K64D triple mutation (FIG. 9E).

Residues G72 and V74 contributed to the binding interface of the hHVEM:hCD160 and hHVEM:hBTLA complexes (FIGS. 2E-2G and FIG. 4B), whereas H86 and L90 resides were within the hHVEM:hLIGHT interface in close proximity to hLIGHT Y173, based on the hHVEM:hLIGHT structure (FIG. 2H and FIG. 4B). The mHVEM G72R-V74A double mutation exhibited no binding to mBTLA or mCD160, while it retained wild-type binding to mLIGHT in the cell-cell and cell-protein interaction system (FIG. 4A and FIGS. 9E-9G). This mHVEM mutant was selected for further analysis and is designated as mHVEM^{-BT/160}, denoting loss of BTLA and CD160 binding. The mHVEM H86D-L90A double mutation showed no binding to mLIGHT and wild-type binding to mBTLA and mCD160 (FIG. 4A and FIGS. 9E-9G). This mHVEM H86D-L90A mutant was thus designated as mHVEM^{-LIGHT}, denoted loss of LIGHT binding. Both mHVEM^{-BT/160} and mHVEM^{-LIGHT} proteins were expressed in soluble form and their ligand binding was measured by surface plasmon resonance. The mHVEM^{-BT/160} eliminated binding to both mBTLA/mCD160 while it still retained close to wild-type binding to mLIGHT (FIG. 4C). The mHVEM^{-LIGHT} had approximately 5-fold and 3-fold reduced binding to mBTLA and mCD160, respectively, but had more than a three log-fold decrease in binding to mLIGHT (FIG. 4C). It was also determined if signaling in vitro by mHVEM muteins was ligand selective. WT mHVEM or mHVEM muteins were co-transfected in 293T cells with an NF-κB-driven luciferase vector. Transfectants of 293T cells with either mCD160 or mLIGHT activated downstream NF-κB signaling of WT mHVEM (FIG. 9G). Activation was selective, however, as mHVEM^{-BT/160} transfectants might be signaled by LIGHT but not by CD160 expressing cells, and the opposite was true for cells expressing mHVEM^{-LIGHT}.

mHVEM-LIGHT Mice were More Susceptible to Yersinia Infection

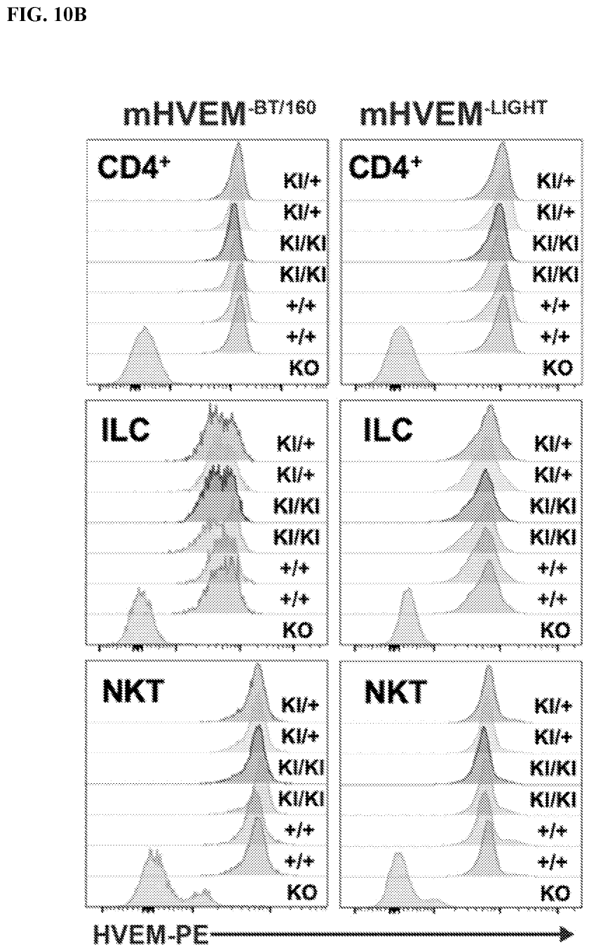

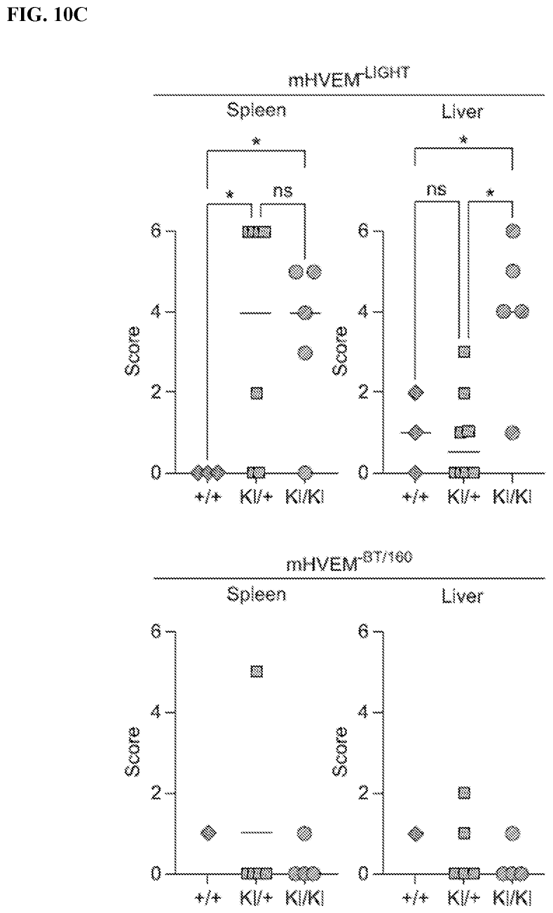

The role of the mouse HVEM muteins was tested, mHVEM^{-BT/160} (G72R-V74A) and mHVEM^{-LIGHT} (H86D-L90A) in vivo. The CRISPR/Cas9 system was utilized to generate two knockin (KI) mouse strains (FIG. 10A). KI homozygous mice having either HVEM mutein were born at the expected frequency with normal size and maturation. Immune cells from homozygous KI mice from either strain expressed a normal surface level of HVEM in different cell types, including splenic CD4⁺ T cells, invariant nature killer T (iNKT) cells, and innate lymphoid cells (ILCs) (FIG. 10B).

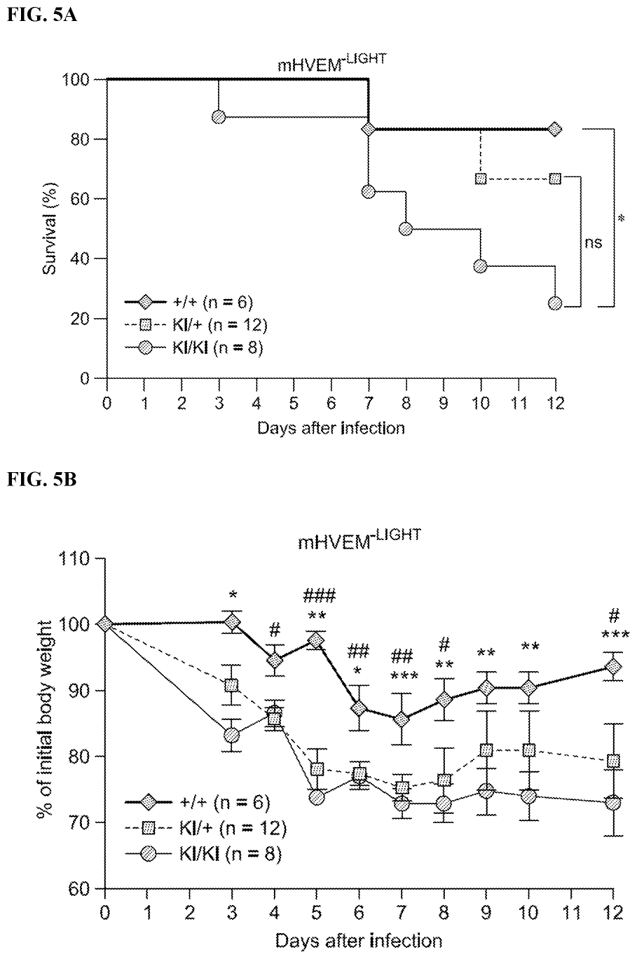

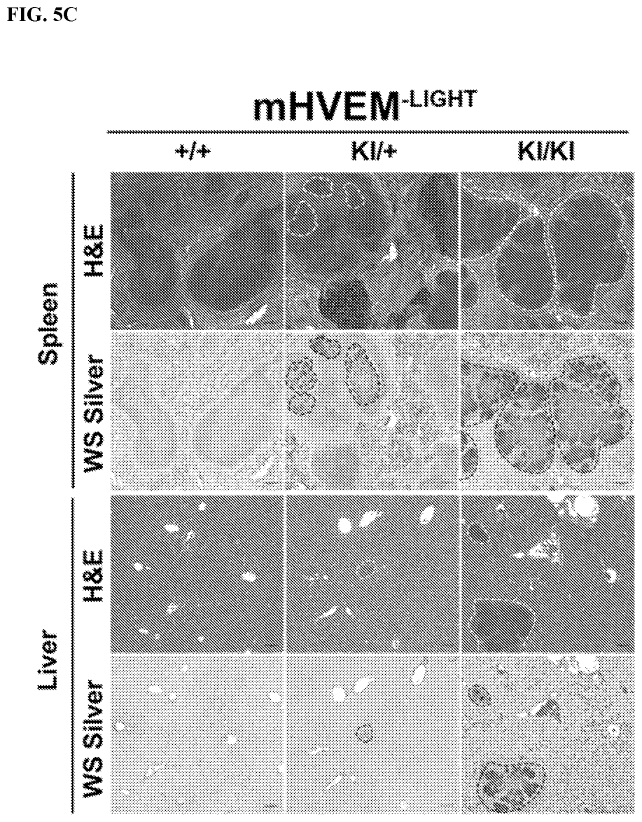

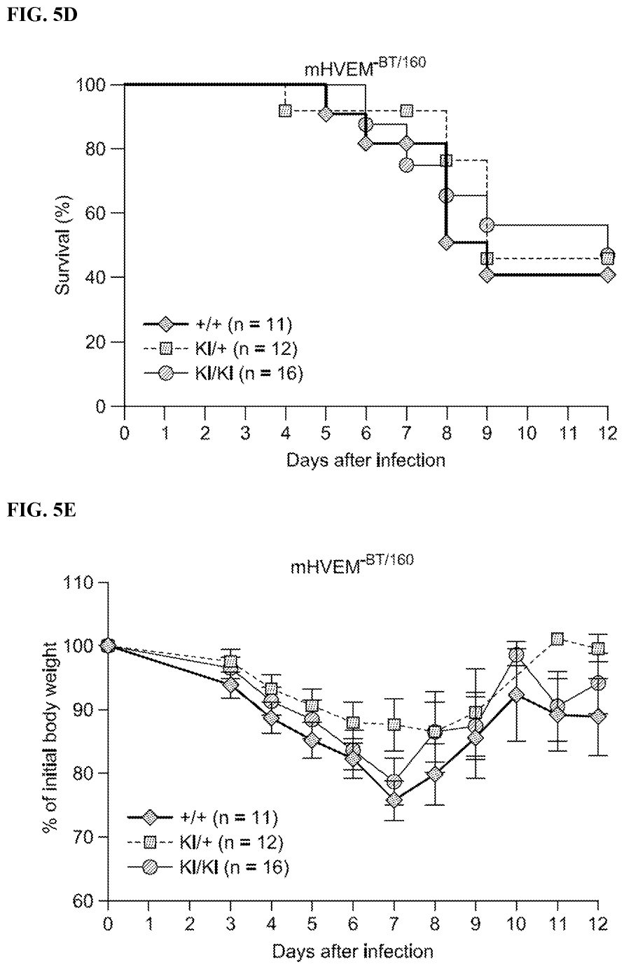

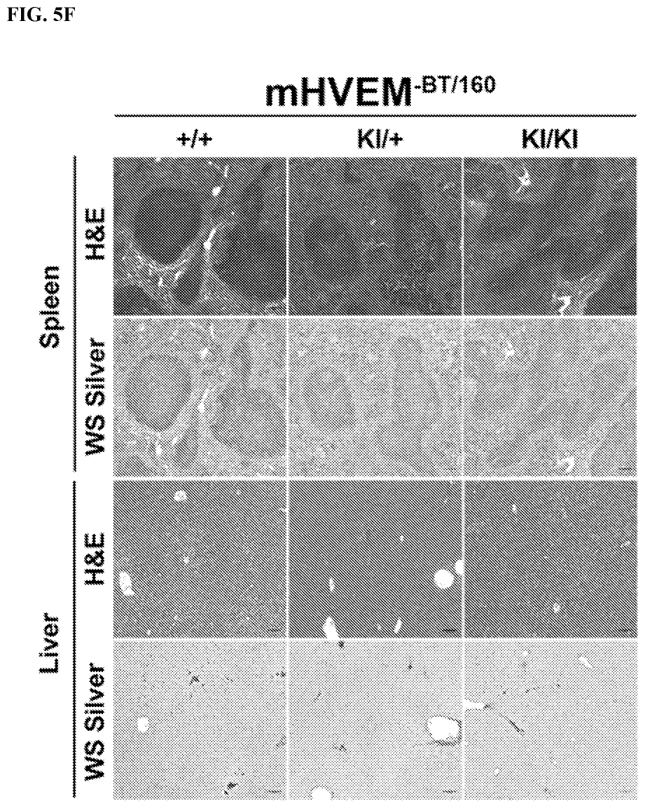

Previously, using conditional HVEM knockouts, it was reported that HVEM signals in ILC3 were critical for host defense against oral infection with Yersinia enterocolitica (Y. enterocolitica) (34). Importantly, the evidence from whole body LIGHT-deficient mice suggested that this HVEM-mediated protection was dependent on LIGHT, not on BTLA or CD160. These data did not exclude a contribution by other aspects of this network. For example, the LTβR deficient mice were not tested and LIGHT-LTβR interactions were also eliminated when the gene encoding LIGHT was deleted. To test the in vivo function of the HVEM muteins, mHVEM^{-BT/160} and mHVEM^{-LIGHT} mice were orally infected with Y. enterocolitica. Homozygous mHVEM^{-LIGHT} (KI/KI) mice displayed lower survival, more pronounced weight loss, and large areas of necrosis in the liver and spleen compared with control WT mice (FIGS. 5A-5C and FIG. 10C). This severe disease outcome was similar to that observed in Light knockout mice (34), indicating the LIGHT-LTβR interactions did not contribute to resistance or cannot overcome the effect of loss of LIGHT binding to HVEM expressed by ILC3. Interestingly, heterozygous mHVEM^{-LIGHT} (KI/+) mice had an intermediate phenotype, with weight loss similar to homozygous mHVEM^{-LIGHT} mice, but they showed better survival than mHVEM^{-LIGHT} mice, as well as reduced necrotic areas and decreased bacterial foci in spleen and liver. Considering that LIGHT binding induced a trimerization of HVEM that likely enhanced signaling, an intermediate phenotype might be expected in KI/+ heterozygous mice that would form fewer WT (wild-type) HVEM trimers. In a separate group of Y. enterocolitica infections carried out with mHVEM^{-BT/160} mice, animals homozygous for a gene encoding the HVEM mutein that did not bind either IgSF ligand responded similarly to WT mice (FIGS. 5D-5F and FIG. 10C). Because of normal experimental variability in bacterial cultures, there was increased weight loss and decreased survival in the WT mice in the series of experiments with mHVEM^{-BT/160} mice (FIGS. 5D-5F) compared to WT controls in experiments with mHVEM^{-LIGHT} mice (FIGS. 5A-5C). The key comparison, however, was mHVEM mutein expressing to WT mice within an experiment, and only mHVEM^{-LIGHT} showed a difference. Also, note that clearance was greatly diminished at day 12 only in mHVEM^{-LIGHT} mice and the recovery from weight loss was complete at the end of the experiment in surviving mHVEM^{-BT/160} mice, similar to the WT controls. Therefore, the data show that LIGHT is the unique ligand for HVEM in protection from Y. enterocolitica and that LIGHT binding to the LTβR was not relevant in this context.

mHVEM^{-BT/160} Mice were More Susceptible to Hepatic Inflammation

Previous studies have reported that Btla^{-/-} or Cd160^{-/-} mice were more susceptible to hepatic injury induced by Concanavalin A (ConA) or by the synthetic glycosphingolipid alpha-galactosylceramide (αGalCer) (16, 17, 24). Studies described herein were focused on αGalCer because of its well-defined mechanism of action as a specific activator of iNKT cells, which were very abundant in intrahepatic lymphocyte populations. When mice were injected with αGalCer, iNKT cells were rapidly stimulated and produced many types of pro-inflammatory cytokines, including the TNF, IFNγ, and IL-4, driving liver injury (Biburger and Tiegs, 2005; 43). Furthermore, both BTLA and CD160 were expressed by iNKT cells and both molecules served to attenuate production of inflammatory cytokines by iNKT cells during αGalCer-induced acute hepatitis (17; 24), provided an example in which two HVEM binding IgSF molecules were required in one cell type. The function of LIGHT in this model had not been reported.

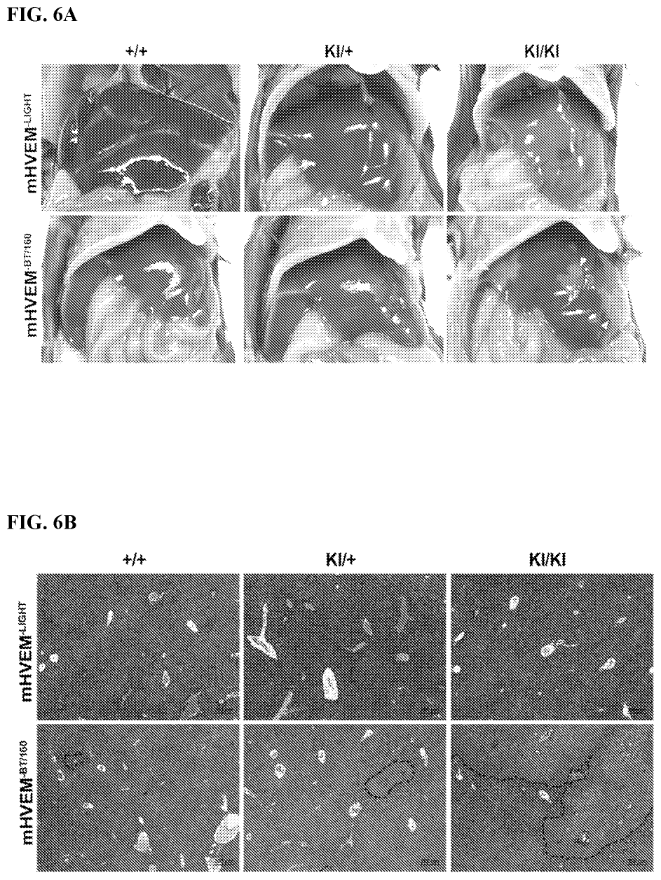

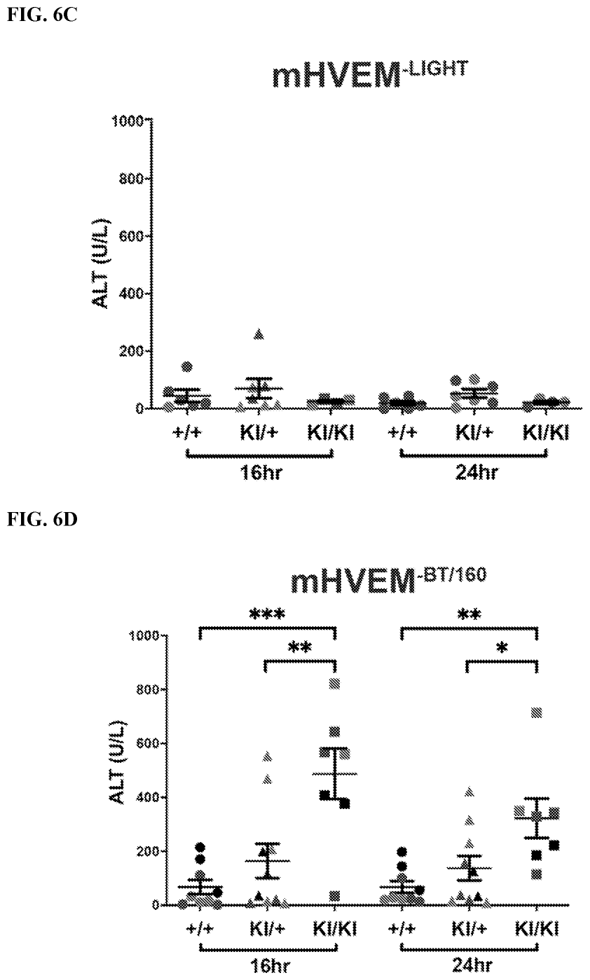

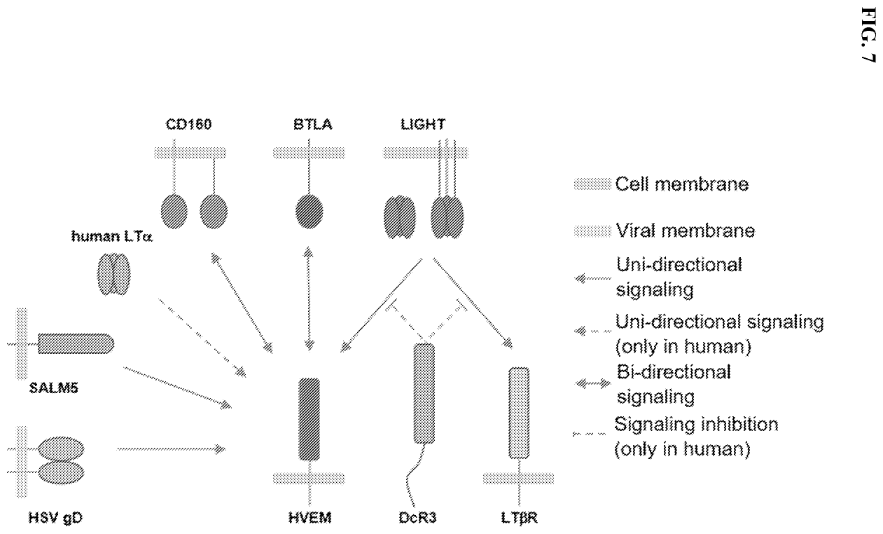

αGalCer was injected into female mHVEM^{-LIGHT} and mHVEM^{-BT/160} mice and controls. mHVEM^{-LIGHT} mice presented with a similar phenotype to controls, which at this dose induced only limited αGalCer-triggered liver damage and serum ALT activity (FIGS. 6A-6C). By contrast, larger light spots on the surface of liver and massive hepatic necrotic regions developed in mHVEM^{-BT/160} mice (FIGS. 6A-6B). Consistently, serum alanine aminotransferase (ALT) activity was elevated in mHVEM^{-BT/160} mice compared with littermate control or heterozygous (KI/+) mice (FIG. 6D). Heterozygous mHVEM^{-BT/160} mice showed an intermediate phenotype, particularly with regard to the ALT measurement. Considering that the IgSF ligand-HVEM interaction was monomeric, this phenotype might reflect HVEM gene haploinsufficiency. These findings suggest that HVEM: BTLA and/or HVEM: CD160 engagement generated negative signaling in iNKT cells, thereby preventing severe αGalCer-induced liver injury and hepatitis.

### Example 3: Discussion

HVEM and its ligands constitute an interacting network of cell surface proteins that affect many aspects of lymphocyte function, as well as the responses of numerous other cells types including eosinophils, keratinocytes, epithelial cells and macrophages in the brain (9; 15; 36; 48). In order to understand how HVEM functions in vivo in this network, and to develop therapeutics based on its mechanisms of action, one important tool is new mouse strains including those that delete HVEM expression in certain cell types (25; 34), mutants that separate HVEM ligand function from HVEM signaling, and expression of HVEM mutants with selective binding to only certain ligands. Herein is disclosed structures of human orthologs of members this network, including the ternary hHVEM:hLIGHT:hCD160 and binary hHVEM:hLIGHT complexes; as well as the structure of mHVEM in isolation. These structures guided mutagenesis studies that identified HVEM muteins with selective ligand binding. Additionally, the inventors have tested these HVEM muteins in vivo in mouse strains. In this way, without eliminating expression of any member of the network, the inventors have shown that that selective HVEM-ligand interactions are responsible for host defense from enteric bacterial infection and for the prevention of liver inflammation.

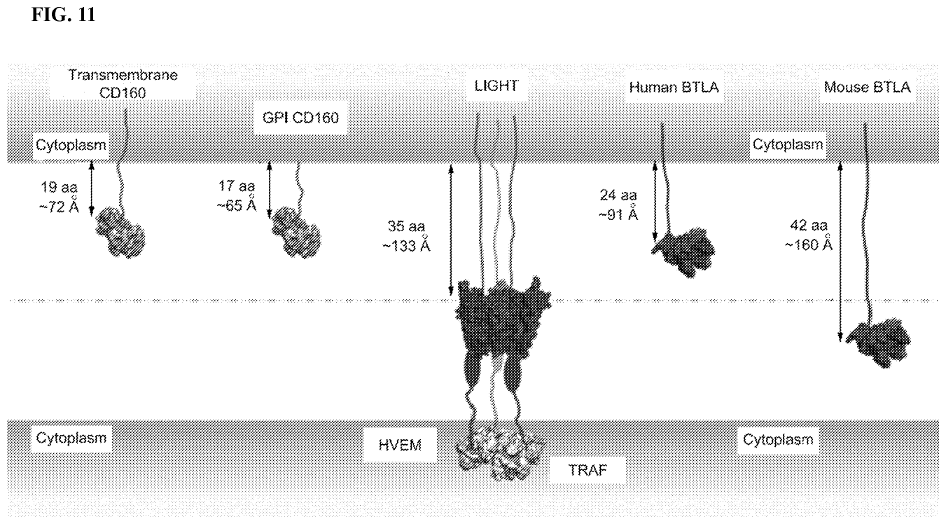

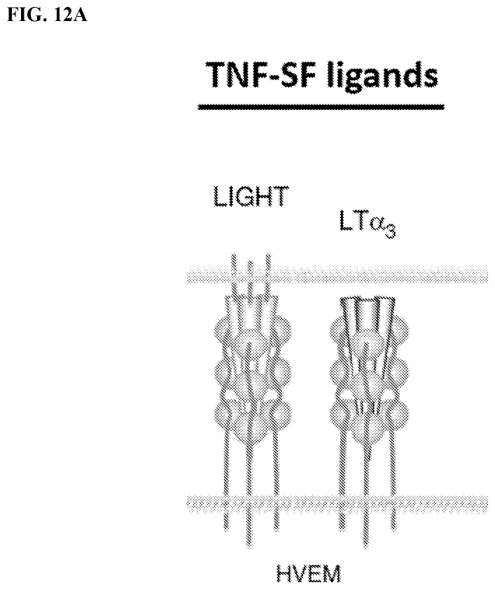

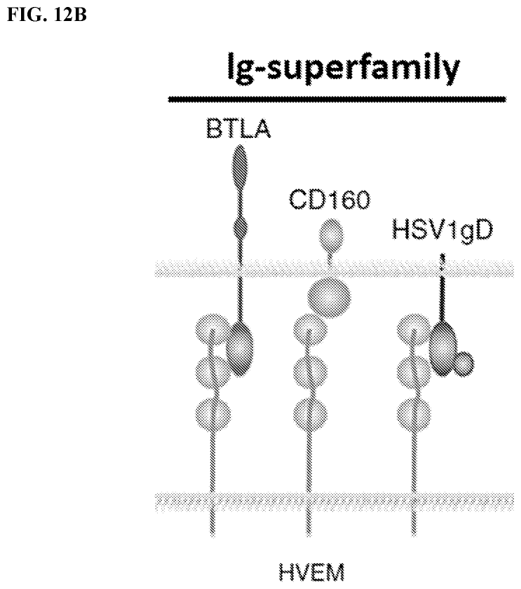

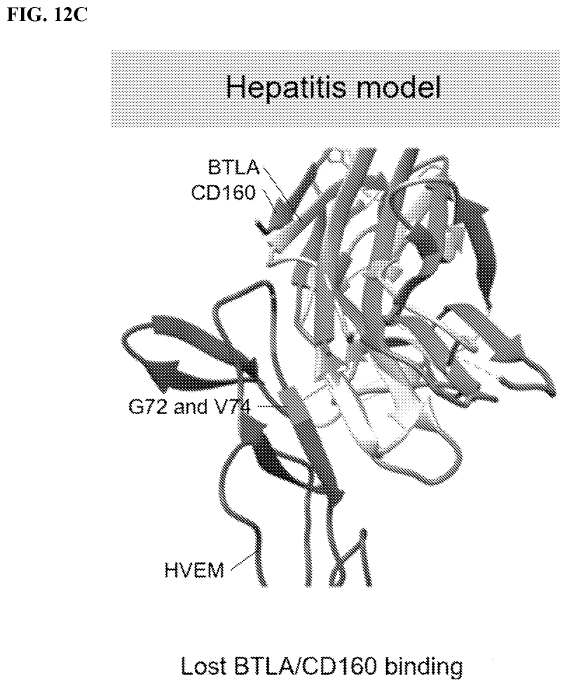

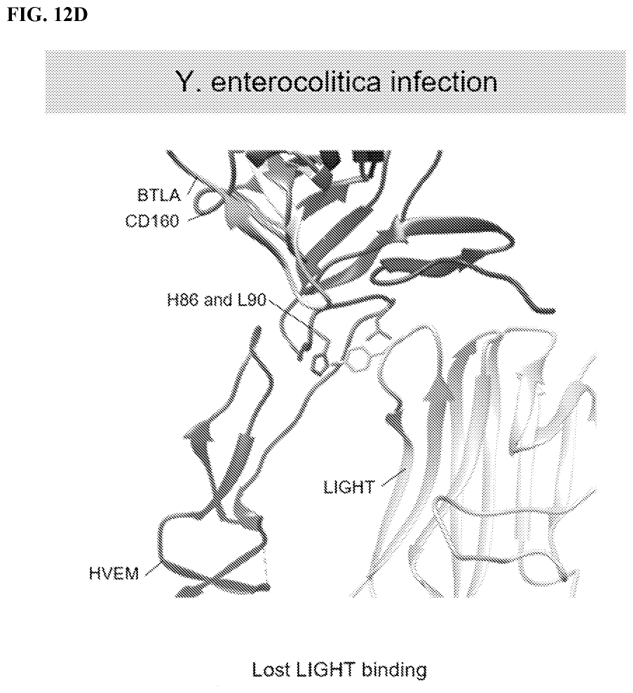

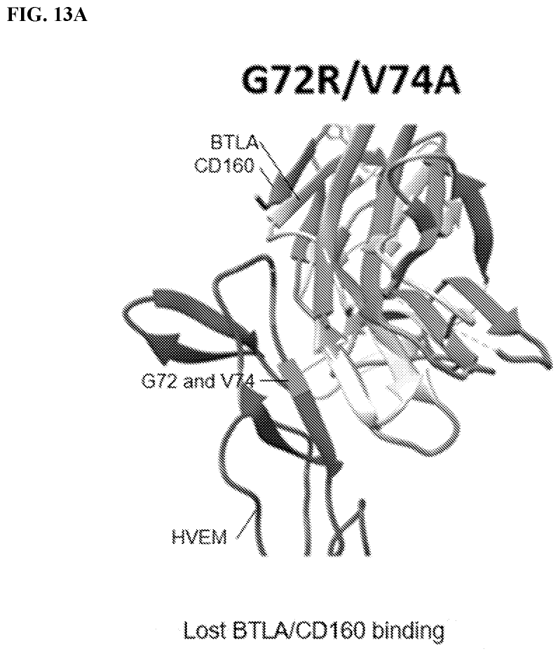

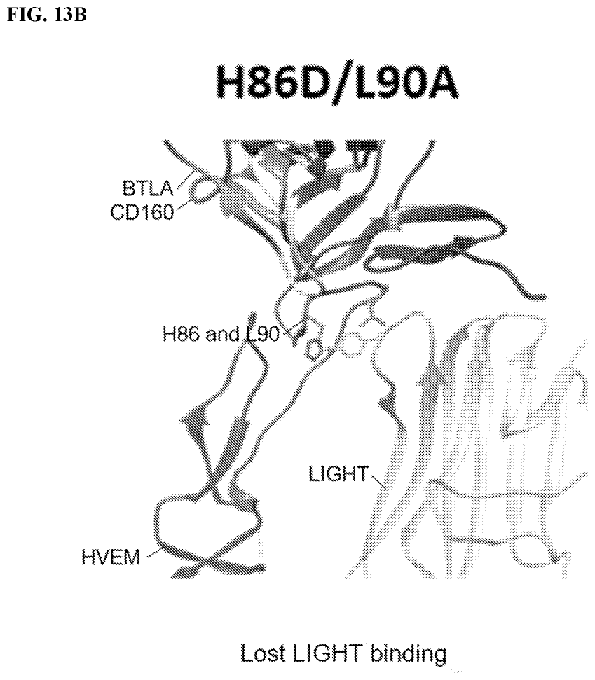

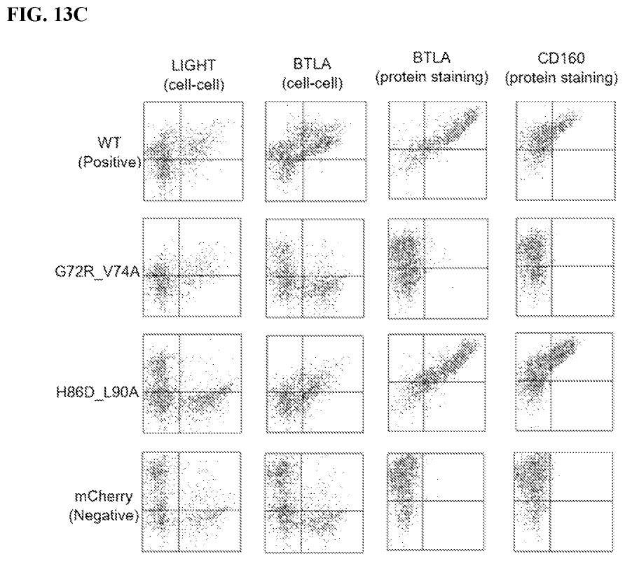

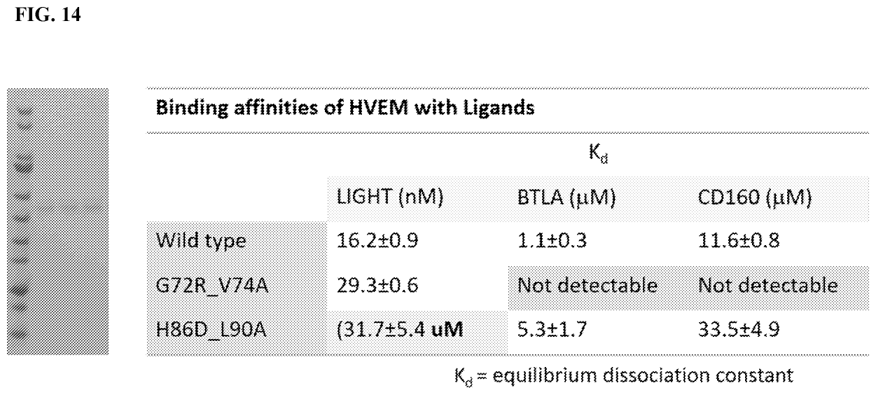

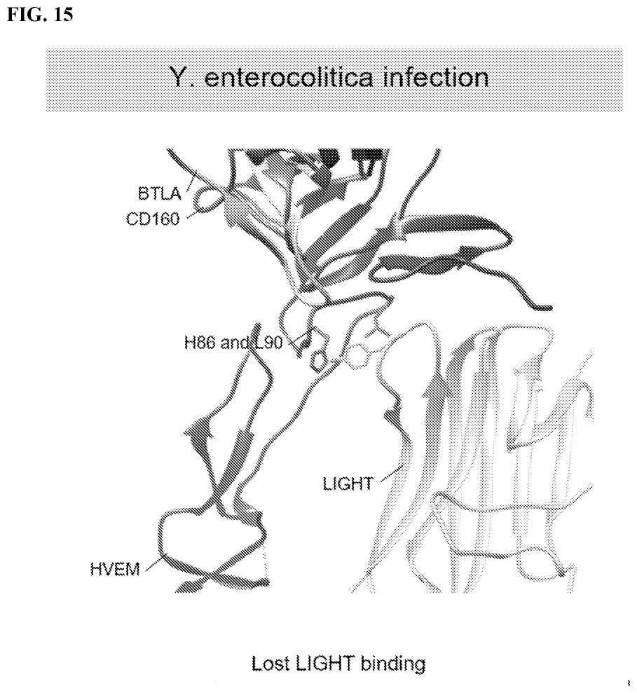

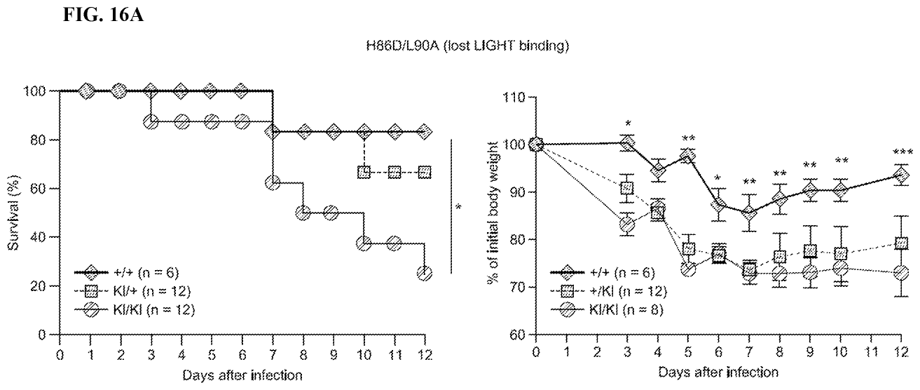

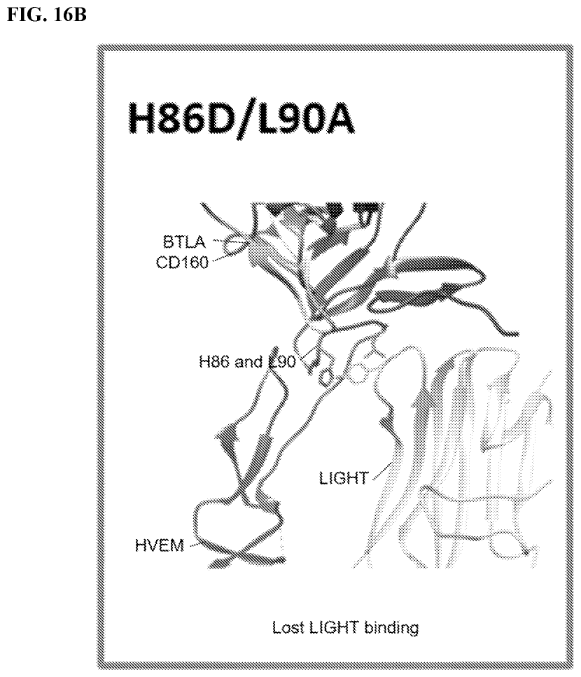

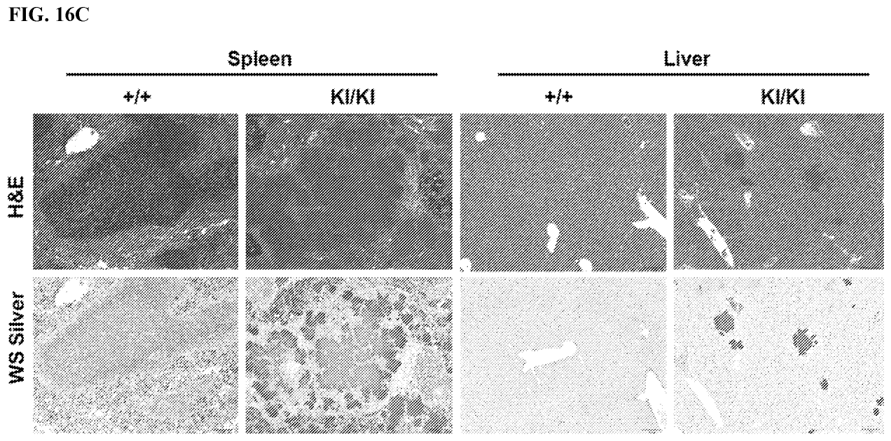

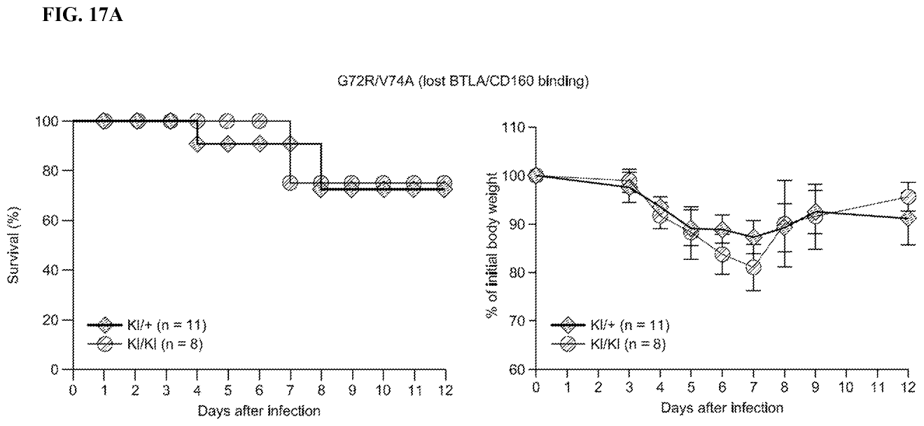

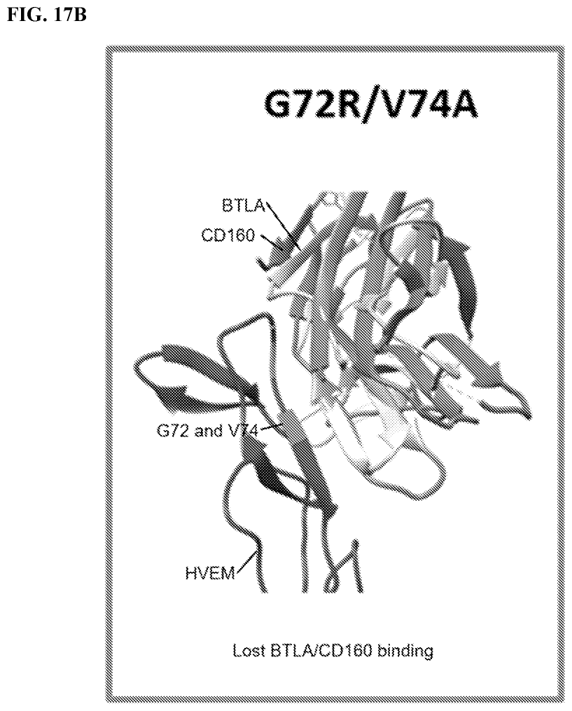

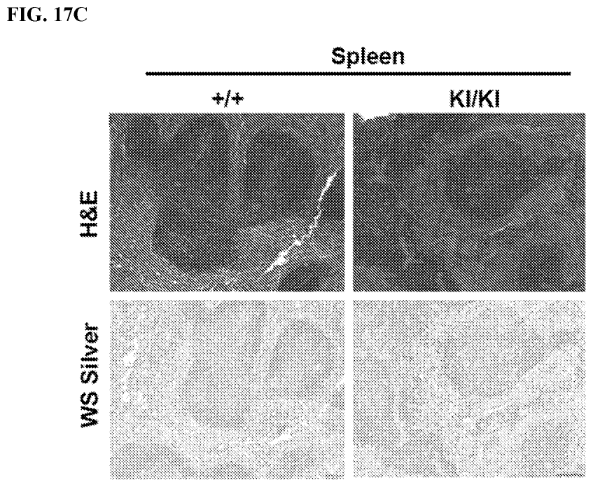

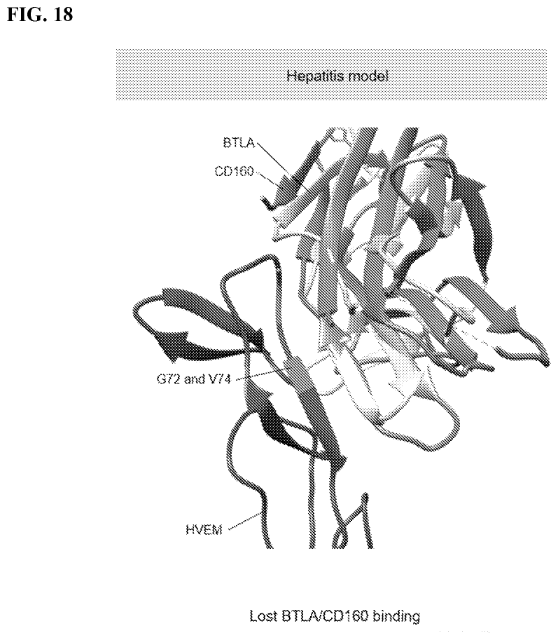

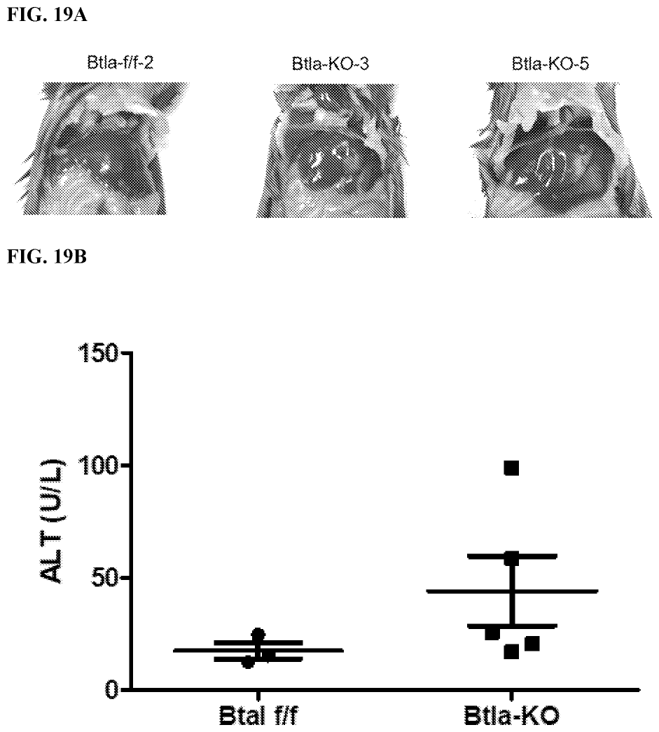

In contrast to the homotrimeric structure of LIGHT, BTLA and CD160 proteins are monomers (7; 48). Crystallographic and biochemical studies illustrated that hHVEM:hBTLA and hHVEM:hCD160 complexes are characterized by a 1:1 stoichiometry (FIGS. 2C-2D) (7). Unlike trimeric LIGHT, which directly drives formation of assemblies containing multiple HVEM molecules, monomeric BTLA and CD160 may activate HVEM receptor to promote NF-κB signaling and cell survival (5, 6) through other mechanisms. The membrane-anchored forms of BTLA and CD160 might drive the localized enrichment of HVEM at cell-cell interfaces, and as a consequence enhance the local concentration of HVEM cytoplasmic domains and associated signaling molecules. Additionally, soluble trimeric LIGHT could contribute by driving the formation of assemblies that bring up to three molecules of HVEM into close proximity, which may facilitate increased local density of HVEM:BTLA and HVEM:CD160 complexes. The recognition interfaces in the ternary hHVEM:hLIGHT:hCD160 complex are similar to those in the binary hHVEM:hCD160 and hHVEM:hLIGHT complexes, suggesting that little molecular accommodation is required for HVEM to simultaneously engage two types of binding partners. It remains to be determined under which conditions HVEM concurrently binds LIGHT and one of its IgSF ligands, if a trimeric HVEM:LIGHT complex can contain mixed IgSF binding partners, both CD160 and BTLA, and importantly, whether these interactions enhance BTLA or CD160 mediated signals. Furthermore, LIGHT can be expressed in membrane bound or soluble forms, and it is not known if the membrane-bound form also can bind HVEM simultaneously with BTLA or CD160. Previously, it was suggested that when LIGHT and BTLA are presented on the same cell membrane, membrane LIGHT might limit BTLA binding in trans due to steric incompatibilities associated with the position of the LIGHT and IgSF binding sites on HVEM relative to the cell membrane (37). In humans, the stalk region of LIGHT is 35 amino acids, while for BTLA it is only 24 amino acids. For hCD160, it is 17 amino acids for the glycosylphosphatidylinositol (GPI)-linked form and 19 amino acids for the transmembrane form. These constraints would position BTLA and CD160 too close to the cell membrane to bind HVEM together with LIGHT (FIG. 11). Therefore, it is possible that the membrane bound and secreted forms of LIGHT could have different impacts on HVEM: BTLA and HVEM: CD160 binding, based on their position relative to the cell membrane, but additional in vitro and in vivo studies will be required to verify this.

Whole body and cell-type-specific gene knock outs have provided important insights into the function of HVEM and its binding partners (25;34). Elimination of expression of one member of this network, however, could have complex effects on others. For example, deletion of LIGHT not only eliminates LIGHT-HVEM interaction, but also the LIGHT-LTβR interaction. It is also possible that LIGHT deletion might provide more LTβR available for binding to LTαß₂, and in humans, blockade of LIGHT may alter the degree of inhibition of TL1A and FasL by DcR3, a decoy receptor not present in mice. Although analysis of no single mutation may discriminate between all these possibilities, a setup was made to test the importance in vivo of pairwise interactions in the HVEM network in a context in which expression of all of the proteins was maintained. To do this, the inventors mutated solvent accessible amino acids in mHVEM that were close to the ligand binding interfaces defined by structural analyses. The inventors succeeded in identifying mHVEM muteins with selective binding in vitro for either LIGHT or for the two IgSF ligands. These HVEM proteins were expressed at normal amounts on cells in genetically altered mouse strains and were tested in vivo following oral infection with Y. enterocolitica and following injection with αGalCer to activate iNKT cells to cause liver inflammation. These data demonstrate a high degree of ligand selectivity in this more complete network. The present study data show that LIGHT-HVEM interactions are required for host defense against Y. enterocolitica. In mice that retain normal expression of LIGHT and HVEM, but in which only the ability of these proteins to interact was greatly diminished, bacteria spread and weight loss were increased and survival was diminished. The phenotype was similar to mice deficient for HVEM in T cells and ILC3, or in whole body knockout mice lacking LIGHT expression. There was no effect on the host response in mice in which HVEM binding to CD160 and BTLA was diminished. Similarly, liver inflammation was dependent on CD160 and/or BTLA interacting with HVEM. As suggested by other studies (16, 17, 24), this behavior may be due to the loss of inhibitory signaling in the iNKT cells that initiate this inflammatory response. It was not greatly dependent on LIGHT binding to HVEM, suggesting LIGHT induced HVEM trimerization is not a major factor in promoting or inhibiting BTLA and CD160 signaling in this system.

### Example 4: Materials and Methods

### Molecular Cloning and Mutagenesis

A portion of the hHVEM gene encoding residues L39-C162 and mHVEM encoding residues Q39-T142 were amplified by PCR and the resulting DNA fragments were digested with endonucleases BglII and AgeI and ligated into plasmid pMT/BiP/V5-His for His-tag fusion protein production in Drosophila S2 cells. DNA fragment encoding the amino acid sequence “HHHHHHG” (SEQ ID NO: 1) fused to hLIGHT (L83-V240) was cloned into pMT/BiP/V5-His. The mCD160 gene encoding residues 301-154H with the C-terminus fused with amino acids “HHHHHHGGGGSGLNDIFEAQKIEWHE” (SEQ ID NO: 2) was cloned into pET3a. The DNA sequences encoding a protein biologic composed of mHVEM residues (Q39-Q206) followed by human IgG1 and a subsequent hexa-His tag sequences were cloned into pcDNA 3.3 vector (Life technologies) using In-fusion HD cloning enzyme premix (Clontech). DNA fragment encoding the amino acid sequence “HHHHHHGG” (SEQ ID NO: 3) fused to the N-terminus of the single chain homotrimeric mLIGHT extracellular domain (G73-V239) connecting by two (GGGGS) 4 (SEQ ID NO: 10) linkers was cloned into pcDNA 3.3 vector (Life technologies).

A DNA fragment encoding residues of L39-V202 of hHVEM was cloned into an engineered pEGFP-N1 vector (Clontech) for expression as a protein fused with a PD-L1 trans-membrane domain followed by the fluorophore eGFP at the C-terminus. The hHVEM mutant library was generated using the QuickChange II Site-Directed Mutagenesis Kit (Agilent Technologies). Full length of WT mHVEM and mutants were cloned into pmCherry-N1 vector (Clontech), respectively. Full length of mBTLA was cloned into pEGFP-N1 vector (Clontech). Full length of mLIGHT was cloned into pIRES2-EGFP vector (Clontech), which contains a subsequent IRES (Internal Ribosome Entry Site) sequence following by a fluorescent EGFP ORF. For the in vitro HVEM signaling assay, full length proteins including mHVEMWT, mHVEM-BT/160, mHVEM-LIGHT, mCD160, and mLIGHT were cloned into pEGFP-N2 vector (Clontech) with a stop codon so they were not expressed as fusion proteins with EGFP.

### Protein Production and Purification

All hHVEM, hLIGHT and mHVEM proteins were expressed and purified as previously described (20). The extracellular domains of hHVEM (L39-C162), hLIGHT (L83-V240) and mHVEM (Q39-T142) were separately cloned into the pMT/BiP/V5-His A vector (Invitrogen) and co-transfected into Drosophila S2 cells with the pCoBlast (Invitrogen) plasmid at a 20:1 ratio. A stable cell line was selected with Blasticidin following the manufacture's protocol (Invitrogen). All hHVEM, hLIGHT and mHVEM expression were induced with copper sulfate (500 μM final concentration). The proteins from filtered culture supernatants were purified by Ni-NTA column (QIAGEN) and size exclusion chromatography (HiLoad Superdex 75; Amersham). The single chain hCD160-hHVEM fusion protein was expressed in Drosophila S2 cells and purified to homogeneity as previously described (19). The mCD160 protein was purified as inclusion bodies and refolded as previously described (19). The expression vectors encoding mHVEM (Q39-Q206) fused with human IgG1 and a subsequent hexa-His tag sequences were transfected into Expi293 (Gibco) cells using ExpiFectamine 293 transfection kit (Gibco) and the resulting proteins were purified using Ni-resins (Qiagen). The vector encoding a hexa-His tag fused to a single chain homotrimeric mLIGHT extracellular domain (G73-V239) connecting by two (GGGGS) 4 (SEQ ID NO: 10) linkers was transfected into Expi293 (Gibco) cells using the ExpiFectamine 293 transfection kit (Gibco) and the resulting proteins were purified using Ni-resins (Qiagen) and size exclusion chromatography (HiLoad Superdex 75; Amersham). The resulting purified mLIGHT proteins were used freshly.

### Cell Culture

Transformed E. coli cells were cultured in LB (Lysogeny Broth) medium supplemented with 100 mg/L Carbenicillin at 37° C. Transfected Drosophila S2 cells were cultured in complete Schneider's Drosophila medium (Life Technologies) supplemented with 10% heat-inactivated fetal bovine serum in the presence of 25 mg/L Blasticidin for establishing stable cell lines. Protein expression in Drosophila S2 cell lines was induced in Express Five SFM medium (Life Technologies) in the presence of 500 mM CuSO4 at 25° C. Expi293 or 293T cells were maintained in DMEM (Corning) with 10% FBS at 37° C. with 5% CO2. The transfected Expi293 cells were cultured at 37° C. with 5% CO2 for flow cytometry analysis or at 30° C. with 5% CO2 for protein expression.

### Crystallization, Structure Determination and Refinement

The purified hHVEM and hLIGHT proteins were concentrated separately and mixed in a 1:1 molar ratio to generate the hHVEM:hLIGHT complex, at a concentration of 3 mg/mL in 10 mM HEPES, pH 7.0 and 150 mM NaCl solution. The resulting hHVEM:hLIGHT complex was crystallized by sitting drop vapor diffusion using 0.5 uL of protein and 0.5 uL of precipitant composed of 0.1 M Bis-Tris, pH5.5, 0.2 M MgCl2 and 9% PEG3350. Crystals were cryo-protected by immersion in crystallization buffer supplemented with 20% of glycerol, and flash-cooled in liquid nitrogen. The purified single chain hCD160-hHVEM proteins and hLIGHT were concentrated separately and mixed in a 1:1 molar ratio to generate the hHVEM:hLIGHT:hCD160 complex at a concentration of 5 mg/mL in 10 mM HEPES, pH 7.0 and 150 mM NaCl solution. The resulting hHVEM:hLIGHT:hCD160 complex was crystallized by sitting drop vapor diffusion using 0.5 uL of protein and 0.5 uL of precipitant composed of 12% (W/V) PEG3350 and 4% (V/V) tacsimate. Crystals were cryo-protected by immersion in crystallization buffer supplemented with 20% ethylene glycerol, and flash-cooled in liquid nitrogen. The purified mHVEM was concentrated to 3 mg/mL in 10 mM HEPES, pH 7.0 and 150 mM NaCl solution and then crystallized by sitting drop vapor diffusion using 0.5 uL of protein and 0.5 uL of precipitant composed of 90% (V/V) solution A with 0.2 M lithium sulfate, 0.1 M sodium acetate/acetic acid, pH4.5, 30% (W/V) PEG 8000 and 10% (V/V) solution B with NDSB-211. Crystals were cryo-protected by immersion in crystallization buffer supplemented with 40% of glycerol, and flash-cooled in liquid nitrogen.

Diffraction data from the hHVEM:hLIGHT complex were collected at Brookhaven National Laboratory (BNL) beamline X29 (Table 3). Diffraction data from hHVEM:hLIGHT:hCD160 complex and mHVEM were collected at Advanced Photon Source Sector 31, Argonne National Laboratory (Table 3). All diffraction data were integrated and scaled with HKL2000 (29). Phases of the hHVEM:hLIGHT complex were calculated by molecular replacement using the existing PDB structures 4KG8 and 4FHQ as the starting models and the software Molrep in the CCP4 package (45). Phases of hHVEM:hLIGHT:hCD160 complex were calculated by molecular replacement using the existing PDB structure 6NG9 and hHVEM:hLIGHT complex (PDB entry 4RSU) as the starting models and the software Molrep in the CCP4 package (45). Phases of mHVEM were calculated by molecular replacement using the existing PDB structure 4FHQ as the starting model and the software Molrep in the CCP4 package (45). Electron density maps were manually inspected and improved using COOT (10). Following several cycles of manual building in COOT and refinement in REFMAC5, the hHVEM:hLIGHT complex Rwork and Rfree converged to 18.4% and 22.6%, respectively (10, 45).

### Mutagenesis Screening

500 ng wild type and mutants of hHVEM-GFP fusion plasmids in 50 μL PBS were mixed with 50 uL of 0.04 M polyethylenimine (PEI), respectively. The mixtures were kept still for 10 min and then added separately to a 24-well plate with each well containing 1 mL of 106/mL HEK293-Freestyle cells (Invitrogen). The transfected cells were cultured by shaking at a speed of 200 rpm at 37° C. for 72 h followed the transfection, and then the cells were collected and resuspended in PBS. Cells from each well were further diluted to 106 cells/mL.

100 uL of the diluted transfected cells were incubated separately with hCD160-6×His tag, hBTLA-6×His tag (R&D systems) and hLIGHT-6×His tag proteins (made by the methods described above) in the mixtures with anti-6×His tag PE-labeled antibody (Abcam) for 20 min on ice. The cells were subsequently spun down, washed once and resuspended in 100 uL PBS buffer containing additional 0.5% BSA and then subjected to flow cytometric analysis. The cells were gated on GFP positive cells to ensure hHVEM expression and analyzed for the percentage of PE positive cells. The binding of wild-type hHVEM was normalized as 1. The relative binding of hHVEM mutants were calculated by comparing the PE positive cell percentage to the control wild-type hHVEM groups. The error bars reflect the results of three independent experiments.

The mHVEM, mBTLA and mLIGHT constructs were transfected into HEK293 FreeStyle (Life technologies) cells using PEI (Linear Polyethylenimine with molecular weight of 25000; Polysciences Inc.). After 2˜3 days, the cells were harvested and diluted to 106/mL. For measuring cell-cell interactions, 100 uL of cells expressing mHVEM-mCherry proteins were mixed with 100 uL of cells expressing mBTLA-EGFP or mLIGHT-IRES-EGFP proteins and then subjected to shaking (900 RPM) at room temperature for 2 h. These cells were further recorded and analyzed by flow cytometry. For protein staining, 100 uL of cells expressing mHVEM-mCherry proteins were mixed with 0.3 ug mBTLA-penta-His-tag/mCD160-biotin proteins and 0.5 ug of green fluorescent anti-His-tag (Abcam; Cat: ab 1206)/Alexa Fluor 488 conjugated streptavidin (Life technologies; Cat: S11223) proteins. The cells were incubated for 30 min with shaking at room temperature and washed once by PBS containing 0.2% BSA (PBS-BSA). The cells were re-suspended in 100 uL of PBS-BSA and analyzed by flow cytometry.

### Octet Bio-Layer Interferometry

For measuring binding affinities, mHVEM-hIgG1 was immobilized on the sensors (ForteBio) and then challenged with different concentrations of mLIGHT, mBTLA or mCD160. The results were exported and then analyzed using Prism 5 (GraphPad Software). Final response curves were generated after subtracting the responses of the control groups. The equilibrium dissociation constants (KD) of the mHVEM-hIgG1 interaction with mLIGHT were calculated based on the response curves by fitting the data to the equation Y=Bmax X/(X+KD) (Y is the averaged maximum response of each experiments. X is the concentration of the analytes and Bmax is the maximum specific binding. The equilibrium dissociation constants (KD) of mHVEM-hIgG1 interaction with mBTLA or mCD160 were calculated based on the 1:1 Langmuir model.

### HVEM Signaling Assay

Plasmid pGL4.32[luc2P/NF-κB-RE/Hygro] (NF-κB-driven firefly luciferase; Promega) and pRL-TK (Renilla luciferase as an internal control; Promega) were co-transfected with mHVEMWT, mHVEM-BT/160, mHVEM-LIGHT, or control (vector only) into 293T cells by TransIT®-LT1 Transfection Reagent (Mirus). 24 h later, transfected cells were co-cultured with control, mCD160-, or mLIGHT-transfected 293T cells. Luciferase activity was measured on the EnVision® 2104 Multimode Plate Reader (PerkinElmer) using the Dual-Glo® Luciferase Assay System kit (Promega) after another 18 h.

Generation of mHVEM Mutant Mice

The mHVEM mutant mice were generated using the CRISPR/Cas9 system. The transgenic mouse core of the UC San Diego Moores Cancer Center injected the sgRNA-Cas9 complex plus a specific single-stranded DNA (ssDNA) homology directed repair (HDR) template into C57BL/6 pronuclear embryos. All materials of the CRISPR/Cas9 system were designed and ordered from Integrated DNA Technologies (IDT, Newark, NJ). Two specific sgRNAs targeted exon 3 of the Tnfrsf14 locus, sgRNA-1 for Tnfrsf14G72R/V74A (mHVEM-BT/160): 5′-CAGGTCTGCAGTGAGCATAC-3′ (SEQ ID NO: 4) and sgRNA-2 for Tnfrsf14H86D/L90A (mHVEM-LIGHT): 5′-ACATATACCGCCCATGCAAA-3′ (SEQ ID NO: 5). Two specific ssDNAs were used as HDR templates, mHVEM-BT/160: 5′-TGGCTGCAGGTTACCATGTGAAGCAGGTCTGCAGTGAGCACACGCGTACAGCGT GTGCCCCCTGTCCCCCACAGACATATACCGCCCATGCA-3′ (SEQ ID NO: 6) and mHVEM-LIGHT: 5′-CAGGCACAGTGTGTGCCCCCTGTCCCCCACAGACATATACAGCGGACGCTAATG GCGCTAGCAAGTGTCTGCCCTGCGGAGTCTGTGATCCAGGTAGGA-3′ (SEQ ID NO: 7). For screening, the inventors created a new restriction enzyme site near the PAM sequence, which did not alter the amino acid sequence. A new MluI or NheI site was thereby created in the knockin genomes of the mHVEM-BT/160 or mHVEM-LIGHT mice, respectively. The F0 founder pups were screened for exon 3 of the Tnfrsf14 locus by enzyme digestion and PCR using the primers Hvem-exon3-F1 (5′-GTACAGTGTTCAGTTCAGGGATAG-3′) (SEQ ID NO: 8) and Hvem-exon3-R1 (5′-AGCAGGAAAGAACCTCTCATTAC-3′) (SEQ ID NO: 9). The Tnfrsf14 exon 3 sequences were cloned and sequenced from each line of founder mice that had undergone HDR repair. The successfully HDR repair F0 founders were first backcrossed to the WT C57BL/6 strain. Germ-line transmission of each line of mHVEM mutant mice (N1) was verified by PCR and restriction enzyme digestion analysis. Testing for potential off-target genes, analyzed by the software from IDT, and homologous sequences were confirmed by PCR using a specific pair of primers on each gene and sequencing at the N1 generation. Six potential off-target genes were examined from mHVEM^{-BT/160} strain and four genes from mHVEM^{-LIGHT} strain. Two and four founders from mHVEM^{-BT/160} or mHVEM^{-LIGHT} strain, respectively, were verified and backcrossed again to the WT C57BL/6 mice. After two backcrosses with C57BL/6 mice, the inventors obtained heterozygous (KI/+) mice (N2) from each mHVEM mutant strain. The inventors obtained homozygous offspring (N2F1) by intercrossing the N2 generation of KI/+ mice. Age and gender matched cohoused littermates were used for experiments. All mice were bred and housed under specific pathogen-free (SPF) conditions in the vivarium of La Jolla Institute for Immunology (LJI) and all animal experimental procedures were approved by the LJI Animal Care and Use Committee.

### Bacterial Infection

Yersinia enterocolitica strain WA-C (pYV::CM) was prepared as described previously (34, 41). Briefly, Yersinia were grown overnight in LB broth at 30° C., and the overnight culture was expanded with fresh medium for 6 h. Bacteria were washed and diluted with PBS. Co-housed male littermates were infected by oral gavage with 1×108 c.f.u. of Y. enterocolitica. Co-housed mice were separated to individual cages after infection and infected mice were analyzed by measurement of body weight daily and tissues were harvested at 7 days after infection for determination of bacterial c.f.u. and histologic analysis as described previously (34). The body weight was not recorded further once a mouse died or lost more than 30% body weight.

### Histopathology Analysis

Several spleen and liver samples were harvested from each strain which had representative symptoms at 7 days after Y. enterocolitica infection, fixed in zinc formalin, routinely embedded into paraffin blocks, cut at 4 μm thickness, and stained with either hematoxylin and eosin (H&E), or by Warthin-Starry (WS) silver method. Slides were scanned with ZEISS Axio Scan.Z1 Digital Slide Scanner (ZEISS). Splenic and hepatic H&E sections were blinded to conditions and provided with both glass slides and whole slide images by a board-certified pathologist familiar with Yersinia infection disease. After reviewing all slides, the pathologist determined there were 6 categories of lesions in each the liver and spleen. For splenic slides: 0, white pulp lymphoid hyperplasia, no Yersinia colonies; 1, focal marginal zone necrosis, no Yersinia colonies; 2, multifocal marginal zone necrosis, no Yersinia colonies; 3, large marginal zone Yersinia colonies with no necrosis/inflammation; 4, multifocal marginal zone Yersinia colonies with some necrosis or splenitis; 5, large marginal zone Yersinia colonies with necrotizing splenitis; 6, large marginal zone Yersinia colonies with abscesses. For hepatic slides: 0, No lesions; 1, minimal hepatitis or hyper cellularity, no Yersinia colonies; 2, mild multifocal necrotizing hepatitis, no Yersinia colonies; 3, moderate multifocal necrotizing hepatitis, no Yersinia colonies; 4, multiple Yersinia colonies, no inflammation; 5, moderate necrotizing hepatitis with Yersinia colonies; 6, marked necrotizing hepatitis with Yersinia colonies. Slides were then reviewed again and assigned a score.

### Hepatic Inflammation

Co-housed female littermates were inoculated with 2 ug αGalCer (KRN7000, Kyowa Kirin Research, La Jolla, California) in a total volume of 200 ul PBS by retro-orbital injection. Serum ALT activity was measured using a colorimetric/fluorometric assay kit (K752, Biovision) at 16 or 24 h after injection. Hepatic tissues were collected, and the necrotic areas were determined using H&E staining at 24 h after αGalCer treatment.

### Statistics Analysis

All data were randomly collected and analyzed using Microsoft Office Excel and GraphPad Prism 8 software. Data were shown as mean with the standard error of the mean (s.e.m.) or standard deviation (s.d.). The detail of statistical analysis methods and the representing number of mice (n) is indicated in each figure legend. Statistical significance is indicated by *P<0.05; **P<0.01; ***P<0.001; ****P<0.0001.

### REFERENCES

1. Bossen, C., K. Ingold, A. Tardivel, J. L. Bodmer, O. Gaide, S. Hertig, C. Ambrose, J. Tschopp, and P. Schneider. 2006. Interactions of tumor necrosis factor (TNF) and TNF receptor family members in the mouse and human. The Journal of biological chemistry 281:13964-13971.
2. Brunetti, G., R. Rizzi, A. Oranger, I. Gigante, G. Mori, G. Taurino, T. Mongelli, G. Colaianni, A. Di Benedetto, R. Tamma, G. Ingravallo, A. Napoli, M. F. Faienza, A. Mestice, P. Curci, G. Specchia, S. Colucci, and M. Grano. 2014. LIGHT/TNFSF14 increases osteoclastogenesis and decreases osteoblastogenesis in multiple myeloma-bone disease. Oncotarget 5:12950-12967.
3. Cai, G., A. Anumanthan, J. A. Brown, E. A. Greenfield, B. Zhu, and G. J. Freeman. 2008. CD160 inhibits activation of human CD4+ T cells through interaction with herpesvirus entry mediator. Nature immunology 9:176-185.
4. Chellan, B., E. P. Koroleva, T. J. Sontag, A. V. Tumanov, Y. X. Fu, G. S. Getz, and C. A. Reardon. 2013. LIGHT/TNFSR14 can regulate hepatic lipase expression by hepatocytes independent of T cells and Kupffer cells. PloS one 8: e54719.
5. Cheung, T. C., L. M. Oborne, M. W. Steinberg, M. G. Macauley, S. Fukuyama, H. Sanjo, C. D'Souza, P. S. Norris, K. Pfeffer, K. M. Murphy, M. Kronenberg, P. G. Spear, and C. F. Ware. 2009a. T cell intrinsic heterodimeric complexes between HVEM and BTLA determine receptivity to the surrounding microenvironment. Journal of immunology (Baltimore, Md.: 1950) 183:7286-7296.
6. Cheung, T. C., M. W. Steinberg, L. M. Oborne, M. G. Macauley, S. Fukuyama, H. Sanjo, C. D'Souza, P. S. Norris, K. Pfeffer, K. M. Murphy, M. Kronenberg, P. G. Spear, and C. F. Ware. 2009b. Unconventional ligand activation of herpesvirus entry mediator signals cell survival. Proceedings of the National Academy of Sciences of the United States of America 106:6244-6249.
7. Compaan, D. M., L. C. Gonzalez, I. Tom, K. M. Loyet, D. Eaton, and S. G. Hymowitz. 2005. Attenuating lymphocyte activity: the crystal structure of the BTLA-HVEM complex. The Journal of biological chemistry 280:39553-39561.
8. Desai, P., G. Abboud, J. Stanfield, P. G. Thomas, J. Song, C. F. Ware, M. Croft, and S. Salek-Ardakani. 2017. HVEM Imprints Memory Potential on Effector CD8 T Cells Required for Protective Mucosal Immunity. J Immunol 199:2968-2975.
9. Doherty, T. A., P. Soroosh, N. Khorram, S. Fukuyama, P. Rosenthal, J. Y. Cho, P. S. Norris, H. Choi, S. Scheu, K. Pfeffer, B. L. Zuraw, C. F. Ware, D. H. Broide, and M. Croft. 2011. The tumor necrosis factor family member LIGHT is a target for asthmatic airway remodeling. Nat Med 17:596-603.
10. Emsley, P., B. Lohkamp, W. G. Scott, and K. Cowtan. 2010. Features and development of Coot. Acta crystallographica. Section D, Biological crystallography 66:486-501.
11. Giles, D. A., S. Zahner, P. Krause, E. Van Der Gracht, T. Riffelmacher, V. Morris, A. Tumanov, and M. Kronenberg. 2018. The Tumor Necrosis Factor Superfamily Members TNFSF14 (LIGHT), Lymphotoxin beta and Lymphotoxin beta Receptor Interact to Regulate Intestinal Inflammation. Front Immunol 9:2585.
12. Harrop, J. A., P. C. McDonnell, M. Brigham-Burke, S. D. Lyn, J. Minton, K. B. Tan, K. Dede, J. Spampanato, C. Silverman, P. Hensley, R. DiPrinzio, J. G. Emery, K. Deen, C. Eichman, M. Chabot-Fletcher, A. Truneh, and P. R. Young. 1998a. Herpesvirus entry mediator ligand (HVEM-L), a novel ligand for HVEM/TR2, stimulates proliferation of T cells and inhibits HT29 cell growth. The Journal of biological chemistry 273:27548-27556.
13. Harrop, J. A., M. Reddy, K. Dede, M. Brigham-Burke, S. Lyn, K. B. Tan, C. Silverman, C. Eichman, R. DiPrinzio, J. Spampanato, T. Porter, S. Holmes, P. R. Young, and A. Truneh. 1998b. Antibodies to TR2 (herpesvirus entry mediator), a new member of the TNF receptor superfamily, block T cell proliferation, expression of activation markers, and production of cytokines. Journal of immunology (Baltimore, Md.: 1950) 161:1786-1794.
14. Hemingway, F., T. G. Kashima, H. J. Knowles, and N. A. Athanasou. 2013. Investigation of osteoclastogenic signalling of the RANKL substitute LIGHT. Experimental and molecular pathology 94:380-385.
15. Herro, R., J. W. Shui, S. Zahner, D. Sidler, Y. Kawakami, T. Kawakami, K. Tamada, M. Kronenberg, and M. Croft. 2018. LIGHT-HVEM signaling in keratinocytes controls development of dermatitis. The Journal of experimental medicine 215:415-422.
16. Iwata, A., N. Watanabe, Y. Oya, T. Owada, K. Ikeda, A.i Suto, S. Kagami, K. Hirose, H. Kanari, S. Kawashima, T. Nakayama, M. Taniguchi, I. Iwamoto, and H. Nakajima. 2010. Protective roles of B and T lymphocyte attenuator in NKT cell-mediated experimental hepatitis. Journal of immunology (Baltimore, Md.: 1950) 184:127-133.
17. Kim, T. J., G. Park, J. Kim, S. A. Lim, J. Kim, K. Im, M. H. Shin, Y. X. Fu, M. L. Del Rio, J. I. Rodriguez-Barbosa, C. Yee, K. S. Suh, S. J. Kim, S. J. Ha, and K. M. Lee. 2019. CD160 serves as a negative regulator of NKT cells in acute hepatic injury. Nat Commun 10:3258.
18. Le Bouteiller, P., A. Barakonyi, J. Giustiniani, F. Lenfant, A. Marie-Cardine, M. Aguerre-Girr, M. Rabot, I. Hilgert, F. Mami-Chouaib, J. Tabiasco, L. Boumsell, and A. Bensussan. 2002. Engagement of CD160 receptor by HLA-C is a triggering mechanism used by circulating natural killer (NK) cells to mediate cytotoxicity. Proceedings of the National Academy of Sciences of the United States of America 99:16963-16968.
19. Liu, W., S. C. Garrett, E. V. Fedorov, U. A. Ramagopal, S. J. Garforth, J. B. Bonanno, and S. C. Almo. 2019. Structural Basis of CD160: HVEM Recognition. Structure 27:1286-1295.e1284.
20. Liu, W., V. Vigdorovich, C. Zhan, Y. Patskovsky, J. B. Bonanno, S. G. Nathenson, and S. C. Almo. 2015. Increased Heterologous Protein Expression in Drosophila S2 Cells for Massive Production of Immune Ligands/Receptors and Structural Analysis of Human HVEM. Molecular biotechnology 57:914-922.
21. Lo, J. C., Y. Wang, A. V. Tumanov, M. Bamji, Z. Yao, C. A. Reardon, G. S. Getz, and Y. X. Fu. 2007. Lymphotoxin beta receptor-dependent control of lipid homeostasis. Science 316:285-288.
22. Maeda, M., C. Carpenito, R. C. Russell, J. Dasanjh, L. L. Veinotte, H. Ohta, T. Yamamura, R. Tan, and F. Takei. 2005. Murine CD160, Ig-like receptor on NK cells and NKT cells, recognizes classical and nonclassical MHC class I and regulates NK cell activation. Journal of immunology (Baltimore, Md.: 1950) 175:4426-4432.
23. Mana, P., D. Linares, D. G. Silva, S. Fordham, S. Scheu, K. Pfeffer, M. Staykova, and E. M. Bertram. 2013. LIGHT (TNFSF14/CD258) is a decisive factor for recovery from experimental autoimmune encephalomyelitis. Journal of immunology (Baltimore, Md.: 1950) 191:154-163.
24. Miller, M. L., Y. Sun, and Y. X. Fu. 2009. Cutting edge: B and T lymphocyte attenuator signaling on NKT cells inhibits cytokine release and tissue injury in early immune responses. Journal of immunology (Baltimore, Md.: 1950) 183:32-36.
25. Mintz, M. A., J. H. Felce, M. Y. Chou, V. Mayya, Y. Xu, J. W. Shui, J. An, Z. Li, A. Marson, T. Okada, C. F. Ware, M. Kronenberg, M. L. Dustin, and J. G. Cyster. 2019. The HVEM-BTLA Axis Restrains T Cell Help to Germinal Center B Cells and Functions as a Cell-Extrinsic Suppressor in Lymphomagenesis. Immunity 51:310-323 e317.
26. Montgomery, R. I., M. S. Warner, B. J. Lum, and P. G. Spear. 1996. Herpes simplex virus-1 entry into cells mediated by a novel member of the TNF/NGF receptor family. Cell 87:427-436.
27. Murphy, T. L., and K. M. Murphy. 2010. Slow down and survive: Enigmatic immunoregulation by BTLA and HVEM. Annu Rev Immunol 28:389-411.
28. Okwor, I., G. Xu, H. Tang, Y. Liang, Y. X. Fu, and J. E. Uzonna. 2015. Deficiency of CD40 Reveals an Important Role for LIGHT in Anti-Leishmania Immunity. Journal of immunology (Baltimore, Md.: 1950) 195:194-202.
29. Otwinowski, Z., and W. Minor. 1997. Processing of X-ray diffraction data. Methods enzymol 276:307-326.
30. Pasero, C., and D. Olive. 2013. Interfering with coinhibitory molecules: BTLA/HVEM as new targets to enhance anti-tumor immunity. Immunol Lett 151:71-75.
31. Petreaca, M. L., D. Do, S. Dhall, D. McLelland, A. Serafino, J. Lyubovitsky, N. Schiller, and M. M. Martins-Green. 2012. Deletion of a tumor necrosis superfamily gene in mice leads to impaired healing that mimics chronic wounds in humans. Wound Repair Regen 20:353-366.
32. Rooney, I. A., K. D. Butrovich, A. A. Glass, S. Borboroglu, C. A. Benedict, J. C. Whitbeck, G. H. Cohen, R. J. Eisenberg, and C. F. Ware. 2000. The lymphotoxin-beta receptor is necessary and sufficient for LIGHT-mediated apoptosis of tumor cells. The Journal of biological chemistry 275:14307-14315.
33. Sedy, J. R., and P. Ramezani-Rad. 2019. HVEM network signaling in cancer. Adv Cancer Res 142:145-186.
34. Seo, G. Y., J. W. Shui, D. Takahashi, C. Song, Q. Wang, K. Kim, Z. Mikulski, S. Chandra, D. A. Giles, S. Zahner, P. H. Kim, H. Cheroutre, M. Colonna, and M. Kronenberg. 2018. LIGHT-HVEM Signaling in Innate Lymphoid Cell Subsets Protects Against Enteric Bacterial Infection. Cell Host Microbe 24:249-260 e244.
35. Shrestha, R., S. C. Garrett-Thomson, W. Liu, S. C. Almo, and A. Fiser. 2020. Redesigning HVEM Interface for Selective Binding to LIGHT, BTLA, and CD160. Structure 28:1197-1205 e1192.
36. Shui, J. W., A. Larange, G. Kim, J. L. Vela, S. Zahner, H. Cheroutre, and M. Kronenberg. 2012. HVEM signalling at mucosal barriers provides host defence against pathogenic bacteria. Nature 488:222-225.
37. Steinberg, M. W., T. C. Cheung, and C. F. Ware. 2011. The signaling networks of the herpesvirus entry mediator (TNFRSF14) in immune regulation. Immunol Rev 244:169-187.
38. Tamada, K., K. Shimozaki, A. I. Chapoval, Y. Zhai, J. Su, S. F. Chen, S. L. Hsieh, S. Nagata, J. Ni, and L. Chen. 2000. LIGHT, a TNF-like molecule, costimulates T cell proliferation and is required for dendritic cell-mediated allogeneic T cell response. Journal of immunology (Baltimore, Md.: 1950) 164:4105-4110.
39. Tan, C. L., M. J. Peluso, J. M. Drijvers, C. M. Mera, S. M. Grande, K. E. Brown, J. Godec, G. J. Freeman, and A. H. Sharpe. 2018. CD160 Stimulates CD8 (+) T Cell Responses and Is Required for Optimal Protective Immunity to Listeria monocytogenes. Immunohorizons 2:238-250.
40. Tiller, G., H. Laumen, P. Fischer-Posovszky, A. Finck, T. Skurk, M. Keuper, U. Brinkmann, M. Wabitsch, D. Link, and H. Hauner. 2011. LIGHT (TNFSF14) inhibits adipose differentiation without affecting adipocyte metabolism. Int J Obes (Lond) 35:208-216.
41. Trulzsch, K., T. Sporleder, E. I. Igwe, H. Russmann, and J. Heesemann. 2004. Contribution of the major secreted yops of Yersinia enterocolitica O: 8 to pathogenicity in the mouse infection model. Infect Immun 72:5227-5234.
42. Tu, T. C., N. K. Brown, T. J. Kim, J. Wroblewska, X. Yang, X. Guo, S. H. Lee, V. Kumar, K. M. Lee, and Y. X. Fu. 2015. CD160 is essential for NK-mediated IFN-gamma production. The Journal of experimental medicine 212:415-429.
43. Wang, H., D. Feng, O. Park, S. Yin, and B. Gao. 2013. Invariant NKT cell activation induces neutrophil accumulation and hepatitis: opposite regulation by IL-4 and IFN-gamma. Hepatology 58:1474-1485.
44. Whitbeck, J. C., C. Peng, H. Lou, R. Xu, S. H. Willis, M. Ponce de Leon, T. Peng, A. V. Nicola, R. I. Montgomery, M. S. Warner, A. M. Soulika, L. A. Spruce, W. T. Moore, J. D. Lambris, P. G. Spear, G. H. Cohen, and R. J. Eisenberg. 1997. Glycoprotein D of herpes simplex virus (HSV) binds directly to HVEM, a member of the tumor necrosis factor receptor superfamily and a mediator of HSV entry. Journal of virology 71:6083-6093.
45. Winn, M. D., C. C. Ballard, K. D. Cowtan, E. J. Dodson, P. Emsley, P. R. Evans, R. M. Keegan, E. B. Krissinel, A. G. Leslie, A. McCoy, S. J. McNicholas, G. N. Murshudov, N. S. Pannu, E. A. Potterton, H. R. Powell, R. J. Read, A. Vagin, and K. S. Wilson. 2011. Overview of the CCP4 suite and current developments. Acta crystallographica. Section D, Biological crystallography 67:235-242.
46. Zhai, Y., R. Guo, T. L. Hsu, G. L. Yu, J. Ni, B. S. Kwon, G. W. Jiang, J. Lu, J. Tan, M. Ugustus, K. Carter, L. Rojas, F. Zhu, C. Lincoln, G. Endress, L. Xing, S. Wang, K. O. Oh, R. Gentz, S. Ruben, M. E. Lippman, S. L. Hsieh, and D. Yang. 1998. LIGHT, a novel ligand for lymphotoxin beta receptor and TR2/HVEM induces apoptosis and suppresses in vivo tumor formation via gene transfer. The Journal of clinical investigation 102:1142-1151.
47. Zhu, M., Y. Yang, Y. Wang, Z. Wang, and Y. X. Fu. 2011. LIGHT regulates inflamed draining lymph node hypertrophy. Journal of immunology (Baltimore, Md.: 1950) 186:7156-7163.
48. Zhu, Y., S. Yao, M. M. Augustine, H. Xu, J. Wang, J. Sun, M. Broadwater, W. Ruff, L. Luo, G. Zhu, K. Tamada, and L. Chen. 2016. Neuron-specific SALM5 limits inflammation in the CNS via its interaction with HVEM. Science advances 2: e1500637.

INFORMAL SEQUENCE LISTING

SEQ ID NO: 1
HHHHHHG
SEQ ID NO: 2
HHHHHHGGGGSGLNDIFEAQKIEWHE
SEQ ID NO: 3
HHHHHHGG
SEQ ID NO: 4
CAGGTCTGCAGTGAGCATAC
SEQ ID NO: 5
ACATATACCGCCCATGCAAA
SEQ ID NO: 6
TGGCTGCAGGTTACCATGTGAAGCAGGTCTGCAGTGAGCACACGCGTACA
GCGTGTGCCCCCTGTCCCCCACAGACATATACCGCCCATGCA
SEQ ID NO: 7
CAGGCACAGTGTGTGCCCCCTGTCCCCCACAGACATATACAGCGGACGCT
AATGGCGCTAGCAAGTGTCTGCCCTGCGGAGTCTGTGATCCAGGTAGGA
SEQ ID NO: 8
GTACAGTGTTCAGTTCAGGGATAG
SEQ ID NO: 9
AGCAGGAAAGAACCTCTCATTAC
SEQ ID NO: 10
GGGGSGGGGSGGGGSGGGGS
SEQ ID NO: 11 (Mouse full length HVEM sequence
(UniProt ID NO: Q80WM9))
MEPLPGWGSAPWSQAPTDNTFRLVPCVFLLNLLQRISAQPSCRQEEFLVG
DECCPMCNPGYHVKQVCSEHTGTVCAPCPPQTYTAHANGLSKCLPCGVCD
PDMGLLTWQECSSWKDTVCRCIPGYFCENQDGSHCSTCLQHTTCPPGQRV
EKRGTHDQDTVCADCLTGTFSLGGTQEECLPWTNCSAFQQEVRRGTNSTD
TTCSSQVVYYVVSILLPLVIVGAGIAGFLICTRRHLHTSSVAKELEPFQE
QQENTIRFPVTEVGFAETEEETASN
SEQ ID NO: 12 (Mouse Protein Signal Peptide)
MEPLPGWGSAPWSQAPTDNTFRLVPCVFLLNLLQRISA
SEQ ID NO: 13 (Mouse HVEM Transmembrane and cyto-
plasmic sequences)
VVSILLPLVIVGAGIAGFLICTRRHLHTSSVAKELEPFQEQQENTIRFPV
TEVGFAETEEETASN
SEQ ID NO: 14 (Human full length HVEM sequence
(UniProt ID NO: Q92956))
MEPPGDWGPPPWRSTPKTDVLRLVLYLTFLGAPCYAPALPSCKEDEYPVG
SECCPKCSPGYRVKEACGELTGTVCEPCPPGTYIAHLNGLSKCLQCQMCD
PAMGLRASRNCSRTENAVCGCSPGHFCIVQDGDHCAACRAYATSSPGQRV
QKGGTESQDTLCQNCPPGTFSPNGTLEECQHQTKCSWLVTKAGAGTSSSH
WVWWFLSGSLVIVIVCSTVGLIICVKRRKPRGDVVKVIVSVQRKRQEAEG
EATVIEALQAPPDVTTVAVEETIPSFTGRSPNH
SEQ ID NO: 15 (Human Protein Signal Peptide)
MEPPGDWGPPPWRSTPKTDVLRLVLYLTFLGAPCYAPA
SEQ ID NO: 16 (Human HVEM Transmembrane and cyto-
plasmic sequences)
WWFLSGSLVIVIVCSTVGLIICVKRRKPRGDVVKVIVSVQRKRQEAEGEA
TVIEALQAPPDVTTVAVEETIPSFTGRSPNH

1. A herpes virus entry mediator (HVEM) protein or fragment thereof, comprising at least one of the following substitutions:
H86D, L90A, L90D, L90R, A76D, T82D, Y83A, T84D, T84F, N88A, N88D, G89D, L94A, L94D at a position corresponding to the amino acid sequence of SEQ ID NO: 11: or
H86D, L90A, L90D, L90R, E76D, T82D, Y83A, 184D, 184F, N88A, N88D, G89D, L94A, or L94D at a position corresponding the amino acid sequence of SEQ ID NO: 14.

2. The HVEM protein or fragment thereof of claim 1, comprising a H86A and L90A substitution, a H86D and L90A substitution, a H86D and L90D substitution, a H86D and L90R substitution, or a H86R and L90A substitution at positions corresponding to the amino acid sequence of SEQ ID NO: 11.

3. The HVEM protein or fragment thereof of claim 1, comprising a H86D and a L90A substitution, a H86D and a L90D substitution, or a H86D and a L90R substitution at a position corresponding the amino acid sequence of SEQ ID NO: 14.

4. (canceled)

5. (canceled)

6. The HVEM protein or fragment thereof of claim 1, comprising an A76D, a H86D and a L90A substitution at positions corresponding to the amino acid sequence of SEQ ID NO: 11: or
a A76D, a H86D and a L90D substitution at positions corresponding to the amino acid sequence of SEQ ID NO: 11.

7. (canceled)

8. (canceled)

9. A nucleic acid encoding the HVEM protein or fragment thereof of claim 1.

10. (canceled)

11. A Chimeric Antigen Receptor (CAR) comprising:
a) an ectodomain of the HVEM protein or fragment thereof of claim 1, and
b) a transmembrane domain.

12-15. (canceled)

16. A nucleic acid encoding the CAR of claim 11.

17. A T-cell comprising the CAR of claim 11.

18. A method of treating or preventing an autoimmune disorder in a subject in need thereof, said method comprising administering to said subject an effective amount of the HVEM protein or fragment thereof of claim 1.

19. A method of treating or preventing inflammation in a subject in need thereof, said method comprising administering to said subject an effective amount of the HVEM protein or fragment thereof of claim 1.

20. The method of claim 19, wherein said inflammation is acute inflammation caused by a viral infection.

21. A herpes virus entry mediator (HVEM) protein or fragment thereof, comprising at least one of the following substitutions:
G72A, G72D, G72R, T73A, T73D, or V74D at a position corresponding to the amino acid sequence of SEQ ID NO: 11: or
G72D, T73A, or V74E at a position corresponding the amino acid sequence of SEQ ID NO: 14.

22. The HVEM protein or fragment thereof of claim 21, comprising a G72A and a V74D substitution, a G72D and a V74A substitution, a G72D and a V74D substitution, a G72D and a V74R substitution, or a G72R and a V74A substitution at positions corresponding to the amino acid sequence of SEQ ID NO: 11.

23. The HVEM protein or fragment thereof of claim 21, comprising a G72D and a V74E substitution at positions corresponding to the amino acid sequence of SEQ ID NO: 14.

24.-27. (canceled)

28. A nucleic acid encoding the HVEM protein or fragment thereof of claim 21.

29. (canceled)

30. A Chimeric Antigen Receptor (CAR) comprising:
a) an ectodomain of the HVEM protein or fragment thereof of claim 21, and
b) a transmembrane domain.

31.-34. (canceled)

35. A nucleic acid encoding the CAR of claim 30.

36. A T-cell comprising a CAR of claim 30.

37. A method of treating or preventing a disease in a subject in need thereof, said method comprising administering to said subject an effective amount of the HVEM protein or fragment thereof of claim 21.

38. The method of claim 37, wherein said disease is cancer or an autoimmune disorder.

39. (canceled)


SEQUENCE LISTING (58 sequences)

SEQ ID NO: 1 - protein, 7 aa, Synthetic construct
HHHHHHG

SEQ ID NO: 2 - protein, 26 aa, Synthetic construct
HHHHHHGGGGSGLNDIFEAQKIEWHE

SEQ ID NO: 3 - protein, 8 aa, Synthetic construct
HHHHHHGG

SEQ ID NO: 4 - DNA, 20 bp, Synthetic construct
  source 1..20: mol_type = other DNA
caggtctgcagtgagcatac

SEQ ID NO: 5 - DNA, 20 bp, Synthetic construct
  source 1..20: mol_type = other DNA
acatataccgcccatgcaaa

SEQ ID NO: 6 - DNA, 92 bp, Synthetic construct
  source 1..92: mol_type = other DNA
tggctgcaggttaccatgtgaagcaggtctgcagtgagcacacgcgtacagcgtgtgccccctgtcccccacagacatataccgcccatgca

SEQ ID NO: 7 - DNA, 99 bp, Synthetic construct
  source 1..99: mol_type = other DNA
caggcacagtgtgtgccccctgtcccccacagacatatacagcggacgctaatggcgctagcaagtgtctgccctgcggagtctgtgatccaggtagga

SEQ ID NO: 8 - DNA, 24 bp, synthetic construct
  source 1..24: mol_type = other DNA
gtacagtgttcagttcagggatag

SEQ ID NO: 9 - DNA, 23 bp, synthetic construct
  source 1..23: mol_type = other DNA
agcaggaaagaacctctcattac

SEQ ID NO: 10 - protein, 20 aa, Synthetic construct
GGGGSGGGGSGGGGSGGGGS

SEQ ID NO: 11 - protein, 275 aa, Mus musculus
MEPLPGWGSAPWSQAPTDNTFRLVPCVFLLNLLQRISAQPSCRQEEFLVGDECCPMCNPGYHVKQVCSEHTGTVCAPCPPQTYTAHANGLSKCLPCGVCDPDMGLLTWQECSSWKDTVCRCIPGYFCENQDGSHCSTCLQHTTCPPGQRVEKRGTHDQDTVCADCLTGTFSLGGTQEECLPWTNCSAFQQEVRRGTNSTDTTCSSQVVYYVVSILLPLVIVGAGIAGFLICTRRHLHTSSVAKELEPFQEQQENTIRFPVTEVGFAETEEETASN

SEQ ID NO: 12 - protein, 38 aa, Mus musculus
MEPLPGWGSAPWSQAPTDNTFRLVPCVFLLNLLQRISA

SEQ ID NO: 13 - protein, 65 aa, Mus musculus
VVSILLPLVIVGAGIAGFLICTRRHLHTSSVAKELEPFQEQQENTIRFPVTEVGFAETEEETASN

SEQ ID NO: 14 - protein, 282 aa, Homo sapiens
MEPPGDWGPPPWRSTPKTDVLRLVLYLTFLGAPCYAPALPSCKEDEYPVGSECCPKCSPGYRVKEACGELTGTVCEPCPPGTYIAHLNGLSKCLQCQMCDPAMGLRASRNCSRTENAVCGCSPGHFCIVQDGDHCAACRAYATSSPGQRVQKGGTESQDTLCQNCPPGTFSPNGTLEECQHQTKCSWLVTKAGAGTSSSHWVWWFLSGSLVIVIVCSTVGLIICVKRRKPRGDVVKVIVSVQRKRQEAEGEATVIEALQAPPDVTTVAVEETIPSFTGRSPN

SEQ ID NO: 15 - protein, 38 aa, Homo sapiens
MEPPGDWGPPPWRSTPKTDVLRLVLYLTFLGAPCYAPA

SEQ ID NO: 16 - protein, 81 aa, Homo sapiens
WWFLSGSLVIVIVCSTVGLIICVKRRKPRGDVVKVIVSVQRKRQEAEGEATVIEALQAPPDVTTVAVEETIPSFTGRSPNH

SEQ ID NO: 17 - protein, 172 aa, Mus musculus
QPSCRQEEFLVGDECCPMCNPGYHVKQVCSEHTGTVCAPCPPQTYTAHANGLSKCLPCGVCDPDMGLLTWQECSSWKDTVCRCIPGYFCENQDGSHCSTCLQHTTCPPGQRVEKRGTHDQDTVCADCLTGTFSLGGTQEECLPWTNCSAFQQEVRRGTNSTDTTCSSQVVYY

SEQ ID NO: 18 - protein, 172 aa, Synthetic construct
QPSCRQEEFLVGDECCPMCNPGYHVKQVCSEHTATVCAPCPPQTYTAHANGLSKCLPCGVCDPDMGLLTWQECSSWKDTVCRCIPGYFCENQDGSHCSTCLQHTTCPPGQRVEKRGTHDQDTVCADCLTGTFSLGGTQEECLPWTNCSAFQQEVRRGTNSTDTTCSSQVVYY

SEQ ID NO: 19 - protein, 172 aa, Synthetic construct
QPSCRQEEFLVGDECCPMCNPGYHVKQVCSEHTDTVCAPCPPQTYTAHANGLSKCLPCGVCDPDMGLLTWQECSSWKDTVCRCIPGYFCENQDGSHCSTCLQHTTCPPGQRVEKRGTHDQDTVCADCLTGTFSLGGTQEECLPWTNCSAFQQEVRRGTNSTDTTCSSQVVYY

SEQ ID NO: 20 - protein, 172 aa, Synthetic construct
QPSCRQEEFLVGDECCPMCNPGYHVKQVCSEHTRTVCAPCPPQTYTAHANGLSKCLPCGVCDPDMGLLTWQECSSWKDTVCRCIPGYFCENQDGSHCSTCLQHTTCPPGQRVEKRGTHDQDTVCADCLTGTFSLGGTQEECLPWTNCSAFQQEVRRGTNSTDTTCSSQVVYY

SEQ ID NO: 21 - protein, 172 aa, Synthetic construct
QPSCRQEEFLVGDECCPMCNPGYHVKQVCSEHTGAVCAPCPPQTYTAHANGLSKCLPCGVCDPDMGLLTWQECSSWKDTVCRCIPGYFCENQDGSHCSTCLQHTTCPPGQRVEKRGTHDQDTVCADCLTGTFSLGGTQEECLPWTNCSAFQQEVRRGTNSTDTTCSSQVVYY

SEQ ID NO: 22 - protein, 172 aa, Synthetic construct
QPSCRQEEFLVGDECCPMCNPGYHVKQVCSEHTGDVCAPCPPQTYTAHANGLSKCLPCGVCDPDMGLLTWQECSSWKDTVCRCIPGYFCENQDGSHCSTCLQHTTCPPGQRVEKRGTHDQDTVCADCLTGTFSLGGTQEECLPWTNCSAFQQEVRRGTNSTDTTCSSQVVYY

SEQ ID NO: 23 - protein, 172 aa, Synthetic construct
QPSCRQEEFLVGDECCPMCNPGYHVKQVCSEHTGTDCAPCPPQTYTAHANGLSKCLPCGVCDPDMGLLTWQECSSWKDTVCRCIPGYFCENQDGSHCSTCLQHTTCPPGQRVEKRGTHDQDTVCADCLTGTFSLGGTQEECLPWTNCSAFQQEVRRGTNSTDTTCSSQVVYY

SEQ ID NO: 24 - protein, 172 aa, Synthetic construct
QPSCRQEEFLVGDECCPMCNPGYHVKQVCSEHTGTVCDPCPPQTYTAHANGLSKCLPCGVCDPDMGLLTWQECSSWKDTVCRCIPGYFCENQDGSHCSTCLQHTTCPPGQRVEKRGTHDQDTVCADCLTGTFSLGGTQEECLPWTNCSAFQQEVRRGTNSTDTTCSSQVVYY

SEQ ID NO: 25 - protein, 172 aa, Synthetic construct
QPSCRQEEFLVGDECCPMCNPGYHVKQVCSEHTGTVCAPCPPQDYTAHANGLSKCLPCGVCDPDMGLLTWQECSSWKDTVCRCIPGYFCENQDGSHCSTCLQHTTCPPGQRVEKRGTHDQDTVCADCLTGTFSLGGTQEECLPWTNCSAFQQEVRRGTNSTDTTCSSQVVYY

SEQ ID NO: 26 - protein, 172 aa, Synthetic construct
QPSCRQEEFLVGDECCPMCNPGYHVKQVCSEHTGTVCAPCPPQTATAHANGLSKCLPCGVCDPDMGLLTWQECSSWKDTVCRCIPGYFCENQDGSHCSTCLQHTTCPPGQRVEKRGTHDQDTVCADCLTGTFSLGGTQEECLPWTNCSAFQQEVRRGTNSTDTTCSSQVVYY

SEQ ID NO: 27 - protein, 172 aa, Synthetic construct
QPSCRQEEFLVGDECCPMCNPGYHVKQVCSEHTGTVCAPCPPQTYDAHANGLSKCLPCGVCDPDMGLLTWQECSSWKDTVCRCIPGYFCENQDGSHCSTCLQHTTCPPGQRVEKRGTHDQDTVCADCLTGTFSLGGTQEECLPWTNCSAFQQEVRRGTNSTDTTCSSQVVYY

SEQ ID NO: 28 - protein, 172 aa, Synthetic construct
QPSCRQEEFLVGDECCPMCNPGYHVKQVCSEHTGTVCAPCPPQTYFAHANGLSKCLPCGVCDPDMGLLTWQECSSWKDTVCRCIPGYFCENQDGSHCSTCLQHTTCPPGQRVEKRGTHDQDTVCADCLTGTFSLGGTQEECLPWTNCSAFQQEVRRGTNSTDTTCSSQVVYY

SEQ ID NO: 29 - protein, 172 aa, Synthetic construct
QPSCRQEEFLVGDECCPMCNPGYHVKQVCSEHTGTVCAPCPPQTYTADANGLSKCLPCGVCDPDMGLLTWQECSSWKDTVCRCIPGYFCENQDGSHCSTCLQHTTCPPGQRVEKRGTHDQDTVCADCLTGTFSLGGTQEECLPWTNCSAFQQEVRRGTNSTDTTCSSQVVYY

SEQ ID NO: 30 - protein, 172 aa, Synthetic construct
QPSCRQEEFLVGDECCPMCNPGYHVKQVCSEHTGTVCAPCPPQTYTAHAAGLSKCLPCGVCDPDMGLLTWQECSSWKDTVCRCIPGYFCENQDGSHCSTCLQHTTCPPGQRVEKRGTHDQDTVCADCLTGTFSLGGTQEECLPWTNCSAFQQEVRRGTNSTDTTCSSQVVYY

SEQ ID NO: 31 - protein, 172 aa, Synthetic construct
QPSCRQEEFLVGDECCPMCNPGYHVKQVCSEHTGTVCAPCPPQTYTAHADGLSKCLPCGVCDPDMGLLTWQECSSWKDTVCRCIPGYFCENQDGSHCSTCLQHTTCPPGQRVEKRGTHDQDTVCADCLTGTFSLGGTQEECLPWTNCSAFQQEVRRGTNSTDTTCSSQVVYY

SEQ ID NO: 32 - protein, 172 aa, Synthetic construct
QPSCRQEEFLVGDECCPMCNPGYHVKQVCSEHTGTVCAPCPPQTYTAHANDLSKCLPCGVCDPDMGLLTWQECSSWKDTVCRCIPGYFCENQDGSHCSTCLQHTTCPPGQRVEKRGTHDQDTVCADCLTGTFSLGGTQEECLPWTNCSAFQQEVRRGTNSTDTTCSSQVVYY

SEQ ID NO: 33 - protein, 172 aa, Synthetic construct
QPSCRQEEFLVGDECCPMCNPGYHVKQVCSEHTGTVCAPCPPQTYTAHANGASKCLPCGVCDPDMGLLTWQECSSWKDTVCRCIPGYFCENQDGSHCSTCLQHTTCPPGQRVEKRGTHDQDTVCADCLTGTFSLGGTQEECLPWTNCSAFQQEVRRGTNSTDTTCSSQVVYY

SEQ ID NO: 34 - protein, 172 aa, Synthetic construct
QPSCRQEEFLVGDECCPMCNPGYHVKQVCSEHTGTVCAPCPPQTYTAHANGDSKCLPCGVCDPDMGLLTWQECSSWKDTVCRCIPGYFCENQDGSHCSTCLQHTTCPPGQRVEKRGTHDQDTVCADCLTGTFSLGGTQEECLPWTNCSAFQQEVRRGTNSTDTTCSSQVVYY

SEQ ID NO: 35 - protein, 172 aa, Synthetic construct
QPSCRQEEFLVGDECCPMCNPGYHVKQVCSEHTGTVCAPCPPQTYTAHANGRSKCLPCGVCDPDMGLLTWQECSSWKDTVCRCIPGYFCENQDGSHCSTCLQHTTCPPGQRVEKRGTHDQDTVCADCLTGTFSLGGTQEECLPWTNCSAFQQEVRRGTNSTDTTCSSQVVYY

SEQ ID NO: 36 - protein, 172 aa, Synthetic construct
QPSCRQEEFLVGDECCPMCNPGYHVKQVCSEHTGTVCAPCPPQTYTAHANGLSKCAPCGVCDPDMGLLTWQECSSWKDTVCRCIPGYFCENQDGSHCSTCLQHTTCPPGQRVEKRGTHDQDTVCADCLTGTFSLGGTQEECLPWTNCSAFQQEVRRGTNSTDTTCSSQVVYY

SEQ ID NO: 37 - protein, 172 aa, Synthetic construct
QPSCRQEEFLVGDECCPMCNPGYHVKQVCSEHTGTVCAPCPPQTYTAHANGLSKCDPCGVCDPDMGLLTWQECSSWKDTVCRCIPGYFCENQDGSHCSTCLQHTTCPPGQRVEKRGTHDQDTVCADCLTGTFSLGGTQEECLPWTNCSAFQQEVRRGTNSTDTTCSSQVVYY

SEQ ID NO: 38 - protein, 172 aa, Synthetic construct
QPSCRQEEFLVGDECCPMCNPGYHVKQVCSEHTATDCAPCPPQTYTAHANGLSKCLPCGVCDPDMGLLTWQECSSWKDTVCRCIPGYFCENQDGSHCSTCLQHTTCPPGQRVEKRGTHDQDTVCADCLTGTFSLGGTQEECLPWTNCSAFQQEVRRGTNSTDTTCSSQVVYY

SEQ ID NO: 39 - protein, 172 aa, Synthetic construct
QPSCRQEEFLVGDECCPMCNPGYHVKQVCSEHTDTACAPCPPQTYTAHANGLSKCLPCGVCDPDMGLLTWQECSSWKDTVCRCIPGYFCENQDGSHCSTCLQHTTCPPGQRVEKRGTHDQDTVCADCLTGTFSLGGTQEECLPWTNCSAFQQEVRRGTNSTDTTCSSQVVYY

SEQ ID NO: 40 - protein, 172 aa, Synthetic construct
QPSCRQEEFLVGDECCPMCNPGYHVKQVCSEHTDTDCAPCPPQTYTAHANGLSKCLPCGVCDPDMGLLTWQECSSWKDTVCRCIPGYFCENQDGSHCSTCLQHTTCPPGQRVEKRGTHDQDTVCADCLTGTFSLGGTQEECLPWTNCSAFQQEVRRGTNSTDTTCSSQVVYY

SEQ ID NO: 41 - protein, 172 aa, Synthetic construct
QPSCRQEEFLVGDECCPMCNPGYHVKQVCSEHTDTRCAPCPPQTYTAHANGLSKCLPCGVCDPDMGLLTWQECSSWKDTVCRCIPGYFCENQDGSHCSTCLQHTTCPPGQRVEKRGTHDQDTVCADCLTGTFSLGGTQEECLPWTNCSAFQQEVRRGTNSTDTTCSSQVVYY

SEQ ID NO: 42 - protein, 172 aa, Synthetic construct
QPSCRQEEFLVGDECCPMCNPGYHVKQVCSEHTRTACAPCPPQTYTAHANGLSKCLPCGVCDPDMGLLTWQECSSWKDTVCRCIPGYFCENQDGSHCSTCLQHTTCPPGQRVEKRGTHDQDTVCADCLTGTFSLGGTQEECLPWTNCSAFQQEVRRGTNSTDTTCSSQVVYY

SEQ ID NO: 43 - protein, 172 aa, Synthetic construct
QPSCRQEEFLVGDECCPMCNPGYHVKQVCSEHTGTVCAPCPPQTYTAAANGASKCLPCGVCDPDMGLLTWQECSSWKDTVCRCIPGYFCENQDGSHCSTCLQHTTCPPGQRVEKRGTHDQDTVCADCLTGTFSLGGTQEECLPWTNCSAFQQEVRRGTNSTDTTCSSQVVYY

SEQ ID NO: 44 - protein, 172 aa, Synthetic construct
QPSCRQEEFLVGDECCPMCNPGYHVKQVCSEHTGTVCAPCPPQTYTADANGASKCLPCGVCDPDMGLLTWQECSSWKDTVCRCIPGYFCENQDGSHCSTCLQHTTCPPGQRVEKRGTHDQDTVCADCLTGTFSLGGTQEECLPWTNCSAFQQEVRRGTNSTDTTCSSQVVYY

SEQ ID NO: 45 - protein, 172 aa, Synthetic construct
QPSCRQEEFLVGDECCPMCNPGYHVKQVCSEHTGTVCAPCPPQTYTADANGDSKCLPCGVCDPDMGLLTWQECSSWKDTVCRCIPGYFCENQDGSHCSTCLQHTTCPPGQRVEKRGTHDQDTVCADCLTGTFSLGGTQEECLPWTNCSAFQQEVRRGTNSTDTTCSSQVVYY

SEQ ID NO: 46 - protein, 172 aa, Synthetic construct
QPSCRQEEFLVGDECCPMCNPGYHVKQVCSEHTGTVCAPCPPQTYTADANGRSKCLPCGVCDPDMGLLTWQECSSWKDTVCRCIPGYFCENQDGSHCSTCLQHTTCPPGQRVEKRGTHDQDTVCADCLTGTFSLGGTQEECLPWTNCSAFQQEVRRGTNSTDTTCSSQVVYY

SEQ ID NO: 47 - protein, 172 aa, Synthetic construct
QPSCRQEEFLVGDECCPMCNPGYHVKQVCSEHTGTVCAPCPPQTYTARANGASKCLPCGVCDPDMGLLTWQECSSWKDTVCRCIPGYFCENQDGSHCSTCLQHTTCPPGQRVEKRGTHDQDTVCADCLTGTFSLGGTQEECLPWTNCSAFQQEVRRGTNSTDTTCSSQVVYY

SEQ ID NO: 48 - protein, 172 aa, Synthetic construct
QPSCRQEEFLVGDECCPMCNPGYHVKQVCSEHTGTVCDPCPPQTYTADANGASKCLPCGVCDPDMGLLTWQECSSWKDTVCRCIPGYFCENQDGSHCSTCLQHTTCPPGQRVEKRGTHDQDTVCADCLTGTFSLGGTQEECLPWTNCSAFQQEVRRGTNSTDTTCSSQVVYY

SEQ ID NO: 49 - protein, 172 aa, Synthetic construct
QPSCRQEEFLVGDECCPMCNPGYHVKQVCSEHTGTVCDPCPPQTYTADANGDSKCLPCGVCDPDMGLLTWQECSSWKDTVCRCIPGYFCENQDGSHCSTCLQHTTCPPGQRVEKRGTHDQDTVCADCLTGTFSLGGTQEECLPWTNCSAFQQEVRRGTNSTDTTCSSQVVYY

SEQ ID NO: 50 - protein, 164 aa, Homo sapiens [sequence omitted]

SEQ ID NO: 51 - protein, 164 aa, Synthetic construct
LPSCKEDEYPVGSECCPKCSPGYRVKEACGELTDTVCEPCPPGTYIAHLNGLSKCLQCQMCDPAMGLRASRNCSRTENAVCGCSPGHFCIVQDGDHCAACRAYATSSPGQRVQKGGTESQDTLCQNCPPGTFSPNGTLEECQHQTKCSWLVTKAGAGTSSSHWV

SEQ ID NO: 52 - protein, 164 aa, Synthetic construct
LPSCKEDEYPVGSECCPKCSPGYRVKEACGELTGAVCEPCPPGTYIAHLNGLSKCLQCQMCDPAMGLRASRNCSRTENAVCGCSPGHFCIVQDGDHCAACRAYATSSPGQRVQKGGTESQDTLCQNCPPGTFSPNGTLEECQHQTKCSWLVTKAGAGTSSSHWV

SEQ ID NO: 53 - protein, 164 aa, Synthetic construct
LPSCKEDEYPVGSECCPKCSPGYRVKEACGELTGTECEPCPPGTYIAHLNGLSKCLQCQMCDPAMGLRASRNCSRTENAVCGCSPGHFCIVQDGDHCAACRAYATSSPGQRVQKGGTESQDTLCQNCPPGTFSPNGTLEECQHQTKCSWLVTKAGAGTSSSHWV

SEQ ID NO: 54 - protein, 164 aa, Synthetic construct
LPSCKEDEYPVGSECCPKCSPGYRVKEACGELTGTVCEPCPPGTYIAHLAGLSKCLQCQMCDPAMGLRASRNCSRTENAVCGCSPGHFCIVQDGDHCAACRAYATSSPGQRVQKGGTESQDTLCQNCPPGTFSPNGTLEECQHQTKCSWLVTKAGAGTSSSHWV

SEQ ID NO: 55 - DNA, 45 bp, Mus musculus
  source 1..45: mol_type = genomic DNA
gtgaagcaggtctgcagtgagcatacaggcacagtgtgtgccccc

SEQ ID NO: 56 - DNA, 45 bp, synthetic construct
  source 1..45: mol_type = other DNA
gtgaagcaggtctgcagtgagcacacgcgtacagcgtgtgccccc

SEQ ID NO: 57 - DNA, 45 bp, Mus musculus
  source 1..45: mol_type = genomic DNA
ccacagacatataccgcccatgcaaatggcctgagcaagtgtctg

SEQ ID NO: 58 - DNA, 45 bp, synthetic construct
  source 1..45: mol_type = other DNA
ccacagacatataccgcggacgctaacggcgctagcaagtgtctg